(19) United States
(12) Patent Application Publication
(10) Pub. No.: US 2023/0174468 A1
(43) Pub. Date: Jun. 8, 2023

(54) SULFATED PILLARARENES, METHODS OF MAKING SAME, AND USES THEREOF

(71) Applicant: University of Maryland, College Park, College Park, MD (US)

(72) Inventors: Lyle David ISAACS, Silver Spring, MD (US); Weijian XUE, College Park, MD (US)

(21) Appl. No.: 17/822,863

(22) Filed: Aug. 29, 2022

Related U.S. Application Data
(63) Continuation-in-part of application No. PCT/US2021/020333, filed on Mar. 1, 2021.
(60) Provisional application No. 63/344,559, filed on May 21, 2022.
(60) Provisional application No. 61/013,336, filed on Dec. 13, 2007.
(60) Provisional application No. 62/982,460, filed on Feb. 27, 2020.

(51) Int. Cl. C07C 305/22; A61P 25/36

(52) U.S. Cl. CPC C07C 305/22; A61P 25/36

(57) ABSTRACT

Provided are sulfated pillararenes and methods of making and using same. The pillararenes have macrocycle core having a plurality of aryl groups, attached (e.g., covalently bonded) in a para orientation to the adjacent methylene groups. The pillararenes have a hydrophobic cavity. The hydrophobic cavity may be used to sequester various materials or to deliver materials sequestered therein.

### CROSS REFERENCE TO RELATED APPLICATION

This application claims priority to U.S. Provisional Application No. 63/344,559, filed on May 21, 2022, and is a continuation-in-part of International Application No. PCT/US2021/020333, filed on Mar. 1, 2021, which claims priority to U.S. Provisional Application No. 62/982,460, filed on Feb. 27, 2020, and to U.S. Provisional Application No. 63/013,336, filed on Apr. 21, 2020, the disclosures of each of which are incorporated by reference herein.

### STATEMENT REGARDING FEDERALLY SPONSORED RESEARCH

This invention was made with government support under CHE1404911 awarded by National Science Foundation. The government has certain rights in the invention.

### BACKGROUND OF THE DISCLOSURE

Several classes of molecular container compounds are known, including cyclodextrins, calixarenes, cyclophanes, pillararenes, and cucurbiturils. These molecular container compounds bind to their target molecules in solution and thereby modulate the properties of the target including optical properties, solubility, odor, and even biological activity. Previous workers in the pillar[n]arene area have synthesized container molecules that feature a hydrophobic cavity and carboxylic acid solubilizing groups and showed that they bind with good affinity toward cationic targets in water. A challenge in the field is how to create new or modify existing molecular containers that maintain good solubility in water and simultaneously enhance their binding affinity toward their targets.

### SUMMARY OF THE DISCLOSURE

The present disclosure provides sulfated pillararenes. The present disclosure also provides methods of making sulfated pillararenes and uses thereof.

In this disclosure, it was shown that, for example, positioning anionic solubilizing groups (e.g., sulfate groups) at the rim of the pillararene cavity significantly enhances their binding affinity toward cationic targets in water and thereby enhances their abilities as sequestering agents for a variety of applications.

In an aspect, the present disclosure provides compounds. The compounds are sulfated pillararenes. A sulfated pillararene comprises a macrocycle core comprising a plurality of aryl groups, where adjacent aryl groups are covalently connected (e.g., linked) via alkyl linking groups (e.g., —CH₂— groups). The alkyl linking groups are para on the aryl groups (e.g., 1,4-phenyl linkages). The linkages may be on different phenyl rings of an aryl group and correspond to a para linkage if the different phenyl rings were superimposed. In various examples, one or more or all of the adjacent aryl group(s) are not covalently connected by alkyl linking groups at meta positions on the aryl groups (e.g., 1,3-phenyl linkages (in the case where the linkages are on different phenyl rings or an aryl group the linkages do not correspond to a meta linkage if the different phenyl rings were superimposed)). Non-limiting examples of sulfated pillararenes are provided herein. Non-limiting examples of methods of making sulfated pillararenes are provided herein.

In an aspect, the present disclosure provides compositions comprising one or more sulfated pillararene(s). Non-limiting examples of compositions are described herein.

A composition may comprise one or more sulfated pillararene(s) and one or more pharmaceutical agent(s). In various examples, a pharmaceutical agent comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

In an aspect, the present disclosure provides uses of sulfated pillararenes. Non-limiting examples of uses of sulfated pillararenes are provided herein.

Sulfated pillararenes can be used to sequester various materials, which may be chemical compounds. In various non-limiting examples, one or more sulfated pillararene(s) is/are used to sequester one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like), drugs of abuse (e.g., methamphetamine, cocaine, fentanyl, carfentanil, and the like), and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescein, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g. tabun, sarin, soman, cyclosarin, 2-(dimethylamino)ethyl N,N-dimethylphosphoramidofluoridate (GV), novichok agents, VE, VG, VM, VX, and the like), and the like); or the like, or a combination thereof.

### BRIEF DESCRIPTION OF THE FIGURES

For a fuller understanding of the nature and objects of the disclosure, reference should be made to the following detailed description taken in conjunction with the accompanying figures.

FIG. 1 shows examples of hosts (sulfated pillararenes).

FIG. 2 shows examples of cationic guests.

FIG. 3 shows examples of drugs of abuse.

FIG. 4 shows examples of neuromuscular blockers.

FIG. 5 shows binding constants for complexes of example hosts with cationic guests.

FIG. 6 shows binding constants for complexes of example hosts with drugs of abuse.

FIG. 7 shows binding constants for complexes of example hosts with neuromuscular blockers.

FIG. 8 shows ¹H NMR spectra recorded (500 MHz, RT, 20 mM phosphate-buffered D₂O) for: a) P[6]AS, b) Methamphetamine, c) an equimolar mixture of P[6]AS and Methamphetamine (0.5 mM), and d) a 2:1 mixture of Methamphetamine (1 mM) and P[6]AS (0.5 mM).

FIG. 9 shows ¹H NMR spectra recorded (500 MHz, RT, 20 mM phosphate-buffered D₂O) for: a) P[6]AS, b) Motor 2, c) Rocuronium, d) an equimolar mixture of P[6]AS and Rocuronium, e) an equimolar mixture of Motor 2 and Rocuronium, f) a mixture of Motor 2 and Rocuronium, then add P[6]AS, g) a mixture of P[6]AS and Rocuronium, then add Motor 2.

FIG. 10 shows a crystal structure of P[6]AS.

FIG. 11 shows a) structure of CB[n] and M2. b) Preparation of Pillar[n]MaxQ (P[5]AS-P[7]AS) and P[5]ACS and the structures of WP[n]. Conditions: a) Py.SO₃, pyridine, 90° C.; b) propane sultone, NaOH, acetone, 8%.

FIG. 12 shows ¹H NMR spectra (600 MHz, D₂O, 298K) recorded for solution of: a) P[6]AS (1 mM), b) guest 25 (1 mM), c) a mixture of P[6]AS (1 mM) and guest 25 (1 mM); d) a mixture of P[6]AS (1 mM) and guest 25 (2 mM).

FIG. 13 shows X-ray crystal structures of P[6]AS and P[5]ACS. a) Cross-eyed stereoview of one molecule of P[6]AS in the unit cell. Views of the packing of P[6]AS in the crystal along the b) z-axis and c) y-axis. d) Cross-eyed stereoview of one molecule of P[5]ACS in the unit cell.

FIG. 14 shows a) a plot of DP versus time from the titration of a mixture of P[6]AS (100 μM) and 17 (500 μM) in the cell with 20 (1 mM) in the syringe. b) Plot of ΔH versus molar ratio of P[6]AS to 20; the solid line represents the best fit of the data to a competitive binding model implemented in the PEAQ-ITC data analysis software with Kₐ=(1.20±0.06)×10¹¹ M⁻¹ and ΔH=−17.1±0.033 kcal mol⁻¹.

FIG. 15 shows ¹H NMR spectra recorded (600 MHz, D₂O, RT) for: a) P[6]AS (1 mM), b) M2 (0.5 mM), c) rocuronium (0.5 mM), d) P[6]AS.rocuronium (0.5 mM), e) M2.rocuronium (0.5 mM), and f) the solution from part e after treatment with 1 equiv. P[6]AS. Proton labelling for M2, P[6]AS, and rocuronium are given in FIG. 11 and FIG. 4.

FIG. 16 shows ¹H NMR spectra (400 MHz, D₂O, RT) recorded for P[5]ACS.

FIG. 17 shows ¹³C NMR spectra (150 MHz, D₂O, EtOH as internal reference, RT) recorded for P[5]ACS.

FIG. 18 shows ¹H NMR spectra (600 MHz, D₂O, RT) recorded for P[5]AS.

FIG. 19 shows ¹³C NMR spectra (150 MHz, D₂O, EtOH as internal reference, RT) recorded for P[5]AS.

FIG. 20 shows ¹H NMR spectra (600 MHz, D₂O, RT) recorded for P[6]AS.

FIG. 21 shows ¹³C NMR spectra (150 MHz, D₂O and CD₃OD 10:1, RT) recorded for P[6]AS.

FIG. 22 shows ¹H NMR spectra (600 MHz, D₂O, RT) recorded for P[7]AS.

FIG. 23 shows ¹³C NMR spectra (150 MHz, D₂O, Dioxane as external reference, RT) recorded for P[7]AS.

FIG. 24 shows ¹H NMR spectra recorded (600 MHz, RT, 20 mM phosphate-buffered D₂O) for: a) P[5]ACS, b) 17, c) an equimolar mixture of P[5]ACS and 17 (1 mM), and d) a 2:1 mixture of 17 (2 mM) and P[5]ACS (1 mM).

FIG. 25 shows ¹H NMR spectra recorded (600 MHz, RT, 20 mM phosphate-buffered D₂O) for: a) P[5]ACS, b) 21, c) an equimolar mixture of P[5]ACS and 21 (1 mM), and d) a 2:1 mixture of 21 (2 mM) and P[5]ACS (1 mM).

FIG. 26 shows ¹H NMR spectra recorded (600 MHz, RT, 20 mM phosphate-buffered D₂O) for: a) P[5]AS, b) 23, c) an equimolar mixture of P[5]AS and 23 (1 mM), and d) a 2:1 mixture of 23 (2 mM) and P[5]AS (1 mM).

FIG. 27 shows ¹H NMR spectra recorded (600 MHz, RT, 20 mM phosphate-buffered D₂O) for: a) P[5]AS, b) 21, c) an equimolar mixture of P[5]AS and 21 (1 mM), and d) a 2:1 mixture of 21 (2 mM) and P[5]AS (1 mM).

FIG. 28 shows ¹H NMR spectra recorded (600 MHz, RT, 20 mM phosphate-buffered D₂O) for: a) P[5]AS, b) 22, c) an equimolar mixture of P[5]AS and 22 (1 mM), and d) a 2:1 mixture of 22 (2 mM) and P[5]AS (1 mM). e) a 3:1 mixture of 22 (3 mM) and P[5]AS (1 mM), and f) a 4:1 mixture of 22 (4 mM) and P[5]AS (1 mM).

FIG. 29 shows ¹H NMR spectra recorded (600 MHz, RT, 20 mM phosphate-buffered D₂O) for: a) P[5]AS, b) 12, c) an equimolar mixture of P[5]AS and 12 (1 mM), and d) a 2:1 mixture of 12 (2 mM) and P[5]AS (1 mM). e) a 3:1 mixture of 12 (3 mM) and P[5]AS (1 mM), and f) a 4:1 mixture of 12 (4 mM) and P[5]AS (1 mM).

FIG. 30 shows ¹H NMR spectra recorded (600 MHz, RT, 20 mM phosphate-buffered D₂O) for: a) P[5]AS, b) 25, c) an equimolar mixture of P[5]AS and 25 (0.5 mM), d) a 2:1 mixture of 25 (1 mM) and P[5]AS (0.5 mM), e) a 3:1 mixture of 25 (1.5 mM) and P[5]AS (0.5 mM), and f) a 4:1 mixture of 25 (2 mM) and P[5]AS (0.5 mM).

FIG. 31 shows ¹H NMR spectra recorded (600 MHz, RT, 20 mM phosphate-buffered D₂O) for: a) P[5]AS, b) 26, c) an equimolar mixture of P[5]AS and 26 (0.5 mM), d) a 2:1 mixture of 26 (1 mM) and P[5]AS (0.5 mM), and e) a 3:1 mixture of 26 (1.5 mM) and P[5]AS (0.5 mM).

FIG. 32 shows ¹H NMR spectra recorded (600 MHz, RT, 20 mM phosphate-buffered D₂O) for: a) P[6]AS, b) 23, c) an equimolar mixture of P[6]AS and 23 (1 mM), and d) a 2:1 mixture of 23 (2 mM) and P[6]AS (1 mM).

FIG. 33 shows ¹H NMR spectra recorded (600 MHz, RT, 20 mM phosphate-buffered D₂O) for: a) P[6]AS, b) 17, c) an equimolar mixture of P[6]AS and 17 (1 mM), and d) a 2:1 mixture of 17 (2 mM) and P[6]AS (1 mM).

FIG. 34 shows ¹H NMR spectra recorded (600 MHz, RT, 20 mM phosphate-buffered D₂O) for: a) P[6]AS, b) 24, c) an equimolar mixture of P[6]AS and 24 (1 mM), and d) a 2:1 mixture of 24 (2 mM) and P[6]AS (1 mM).

FIG. 35 shows ¹H NMR spectra recorded (600 MHz, RT, 20 mM phosphate-buffered D₂O) for: a) P[6]AS, b) 11, c) an equimolar mixture of P[6]AS and 11 (1 mM), and d) a 2:1 mixture of 11 (2 mM) and P[6]AS (1 mM).

FIG. 36 shows ¹H NMR spectra recorded (600 MHz, RT, 20 mM phosphate-buffered D₂O) for: a) P[6]AS, b) 12, c) an equimolar mixture of P[6]AS and 12 (1 mM), and d) a 2:1 mixture of 12 (2 mM) and P[6]AS (1 mM).

FIG. 37 shows ¹H NMR spectra recorded (600 MHz, RT, 20 mM phosphate-buffered D₂O) for: a) P[6]AS, b) 21, c) an equimolar mixture of P[6]AS and 21 (1 mM), and d) a 2:1 mixture of 21 (2 mM) and P[6]AS (1 mM).

FIG. 38 shows ¹H NMR spectra recorded (600 MHz, RT, 20 mM phosphate-buffered D₂O) for: a) P[6]AS, b) 22, c) an equimolar mixture of P[6]AS and 22 (1 mM), and d) a 2:1 mixture of 22 (2 mM) and P[6]AS (1 mM).

FIG. 39 shows ¹H NMR spectra recorded (600 MHz, RT, 20 mM phosphate-buffered D₂O) for: a) P[6]AS, b) 26, c) an equimolar mixture of P[6]AS and 26 (0.5 mM), and d) a 2:1 mixture of 26 (1 mM) and P[6]AS (0.5 mM).

FIG. 40 shows ¹H NMR spectra recorded (600 MHz, RT, 20 mM phosphate-buffered D₂O) for: a) P[7]AS, b) 11, c) an equimolar mixture of P[7]AS and 11 (0.5 mM), and d) a 2:1 mixture of 11 (1 mM) and P[7]AS (0.5 mM).

FIG. 41 shows ¹H NMR spectra recorded (600 MHz, RT, 20 mM phosphate-buffered D₂O) for: a) P[7]AS, b) 17, c) an equimolar mixture of P[7]AS and 17 (0.5 mM), and d) a 2:1 mixture of 17 (1 mM) and P[7]AS (0.5 mM).

FIG. 42 shows ¹H NMR spectra recorded (600 MHz, RT, 20 mM phosphate-buffered D₂O) for: a) P[7]AS, b) 23, c) an equimolar mixture of P[7]AS and 23 (0.5 mM), and d) a 2:1 mixture of 23 (1 mM) and P[7]AS (0.5 mM).

FIG. 43 shows ¹H NMR spectra recorded (600 MHz, RT, 20 mM phosphate-buffered D₂O) for: a) P[7]AS, b) 21, c) an equimolar mixture of P[7]AS and 21 (0.5 mM), and d) a 2:1 mixture of 21 (1 mM) and P[7]AS (0.5 mM).

FIG. 44 shows ¹H NMR spectra recorded (600 MHz, RT, 20 mM phosphate-buffered D₂O) for: a) P[7]AS, b) 22, c) an equimolar mixture of P[7]AS and 22 (0.5 mM), and d) a 2:1 mixture of 22 (1 mM) and P[7]AS (0.5 mM).

FIG. 45 shows ¹H NMR spectra recorded (600 MHz, RT, 20 mM phosphate-buffered D₂O) for: a) P[5]AS, b) Acetylcholine, c) an equimolar mixture of P[5]AS and Acetylcholine (0.5 mM), and d) a 2:1 mixture of Acetylcholine (1 mM) and P[5]AS (0.5 mM).

FIG. 46 shows ¹H NMR spectra recorded (600 MHz, RT, 20 mM phosphate-buffered D₂O) for: a) P[5]AS, b) Rocuronium, c) an equimolar mixture of P[5]AS and Rocuronium (0.5 mM), d) a 2:1 mixture of Rocuronium (1 mM) and P[5]AS (0.5 mM), and e) a 3:1 mixture of Rocuronium (1.5 mM) and P[5]AS (0.5 mM).

FIG. 47 shows ¹H NMR spectra recorded (600 MHz, RT, 20 mM phosphate-buffered D₂O) for: a) P[5]AS, b) Vecuronium, c) an equimolar mixture of P[5]AS and Vecuronium (0.5 mM), d) a 2:1 mixture of Vecuronium (1 mM) and P[5]AS (0.5 mM), and e) a 3:1 mixture of Vecuronium (1.5 mM) and P[5]AS (0.5 mM).

FIG. 48 shows ¹H NMR spectra recorded (600 MHz, RT, 20 mM phosphate-buffered D₂O) for: a) P[5]AS, b) Pancuronium, c) an equimolar mixture of P[5]AS and Pancuronium (0.5 mM), d) a 2:1 mixture of Pancuronium (1 mM) and P[5]AS (0.5 mM), and e) a 3:1 mixture of Pancuronium (1.5 mM) and P[5]AS (0.5 mM).

FIG. 49 shows ¹H NMR spectra recorded (600 MHz, RT, 20 mM phosphate-buffered D₂O) for: a) P[6]AS, b) Vecuronium, c) an equimolar mixture of P[6]AS and Vecuronium (0.5 mM), and d) a 2:1 mixture of Vecuronium (1 mM) and P[6]AS (0.5 mM).

FIG. 50 shows ¹H NMR spectra recorded (600 MHz, RT, 20 mM phosphate-buffered D₂O) for: a) P[6]AS, b) Acetylcholine, c) an equimolar mixture of P[6]AS and Acetylcholine (0.5 mM), and d) a 2:1 mixture of Acetylcholine (1 mM) and P[6]AS (0.5 mM).

FIG. 51 shows ¹H NMR spectra recorded (600 MHz, RT, 20 mM phosphate-buffered D₂O) for: a) P[6]AS, b) Rocuronium, c) an equimolar mixture of P[6]AS and Rocuronium (0.5 mM), and d) a 2:1 mixture of Rocuronium (1 mM) and P[6]AS (0.5 mM).

FIG. 52 shows ¹H NMR spectra recorded (600 MHz, RT, 20 mM phosphate-buffered D₂O) for: a) P[6]AS, b) Pancuronium, c) an equimolar mixture of P[6]AS and Pancuronium (0.5 mM), and d) a 2:1 mixture of Pancuronium (1 mM) and P[6]AS (0.5 mM).

FIG. 53 shows ¹H NMR spectra recorded (600 MHz, RT, 20 mM phosphate-buffered D₂O) for: a) P[7]AS, b) Vecuronium, c) an equimolar mixture of P[7]AS and Vecuronium (0.5 mM), and d) a 2:1 mixture of Vecuronium (1 mM) and P[7]AS (0.5 mM).

FIG. 54 shows ¹H NMR spectra recorded (600 MHz, RT, 20 mM phosphate-buffered D₂O) for: a) P[7]AS, b) Rocuronium, c) an equimolar mixture of P[7]AS and Rocuronium (0.5 mM), and d) a 2:1 mixture of Rocuronium (1 mM) and P[7]AS (0.5 mM).

FIG. 55 shows ¹H NMR spectra recorded (600 MHz, RT, 20 mM phosphate-buffered D₂O) for: a) P[7]AS, b) Pancuronium, c) an equimolar mixture of P[7]AS and Pancuronium (0.5 mM), and d) a 2:1 mixture of Pancuronium (1 mM) and P[7]AS (0.5 mM).

FIG. 56 shows ¹H NMR spectra recorded (600 MHz, RT, 20 mM phosphate-buffered D₂O) for: a) P[7]AS, b) Cisatracurium, c) a 1:4 mixture of Cisatracurium (0.125 mM) and P[7]AS (0.5 mM), d) a 1:2 mixture of Cisatracurium (0.25 mM) and P[7]AS (0.5 mM), e) an equimolar mixture of P[7]AS and Cisatracurium (0.5 mM).

FIG. 57 shows ¹H NMR spectra (600 MHz, D₂O, 298K) recorded for the dilution of host P[5]AS (20.0-0.1 mM). Host P[5]AS is weakly self-associated in water, which is evidenced by the upfield chemical shift changes of the aromatic region at 7.33-7.40 ppm protons.

FIG. 58 shows a plot of chemical shift of P[5]AS versus [P[5]AS]. The solid line represents the best non-linear fitting of the data to a two-fold self-association model with Kₐ=19.7 M⁻¹.

FIG. 59 ¹H NMR spectra (600 MHz, D₂O, 298K) recorded for the dilution of host P[6]AS (20.0-0.1 mM). Host P[6]AS is weakly self-associated in water, which is evidenced by the upfield chemical shift changes of the aromatic region at 7.34-7.38 ppm protons.

FIG. 60 shows a plot of chemical shift of P[6]AS versus [P[6]AS]. The solid line represents the best non-linear fitting of the data to a two-fold self-association model with Kₐ=16.2 M⁻¹.

FIG. 61 shows ¹H NMR spectra (400 MHz, D₂O) recorded for Rim-P[5]AS.

FIG. 62 shows ¹³C NMR spectra (150 MHz, D₂O, EtOH as internal reference) recorded for Rim-P[5]AS.

FIG. 63 shows HepG2 toxicology assays. AK (A,C) and MTS assays (B,D) performed after the cells had been incubated with indicated containers for 24 h. UT=untreated control; Stx=staurosporine.

FIG. 64 shows HEK293 toxicology assays. AK (A,C) and MTS assays (B,D) performed after the cells had been incubated with indicated containers for 24 h. UT=untreated control; Stx=staurosporine.

FIG. 65 shows MTD study performed for P[6]AS. Female Swiss Webster mice (n=5 per group) were dosed via tail vein on days 0 and 2 (denoted by *) with different concentrations of P[6]AS or phosphate buffered saline (PBS). The normalized average weight change per study group is indicated. Error bars represent SEM.

FIG. 66 shows in vivo reversal of methamphetamine-induced hyperlocomotion by P[6]AS. Average locomotion counts for male Swiss Webster mice (n=8; avg weight (g)±SD: 39±2.203) are plotted as a function of treatment. Treatment order was counterbalanced across days, and mice only received one treatment per day. Over six consecutive days of testing mice each received a single treatment of PBS (PBS; 0.01 M; 0.2 mL infused), P[6]AS only (P[6]AS; 4 mM; 0.178 mL infused), methamphetamine only (METH; 0.5 mg/kg; 0.022 mL infused), a premixed solution of P[6]AS and methamphetamine (Premix; ˜7:1 P[6]AS:Meth; 0.178 mL P[6]AS+0.022 mL Meth infused), P[6]AS followed by methamphetamine administered 30 s later (Blocking; 0.178 mL P[6]AS, 0.022 mL Meth infused), and methamphetamine followed by P[6]AS administered 30 s later (Reversal; 0.022 mL Meth, 0.178 mL P[6]AS infused). Bars represent average locomotion counts. Error bars represent the standard error of the mean (SEM). Dots represent counts for each mouse (n=8). Presented p-values are only for significant (p<0.05) Tukey-corrected post-hoc comparisons.

FIG. 67 shows in vivo reversal of methamphetamine-induced hyperlocomotion effects observed after 5 minute delay between treatment with methamphetamine and P[6]AS administration. On day 7 and 8 mice (n=8) received methamphetamine followed by an infusion of 0.01M PBS administered 5 minutes later (REV-C; 0.022 mL Meth, 0.2 mL PBS infused) or methamphetamine followed by P[6]AS administered 5 minutes later (REV-5; 0.022 mL Meth, 0.178 mL P[6]AS infused) in counterbalanced manner. Administration of P[6]AS 5 minutes after exposure to methamphetamine reduced hyperlocomotion (paired t-test, t(7)=2.757, p=0.0282). Bars represent average locomotion counts. Error bars represent the standard error of the mean (SEM). Dots represent counts for each mouse (n=8).

FIG. 68 shows the chemical structures for MDMA, mephedrone, heroin, and methamphetamine.

FIG. 69 shows a) a plot of DP vs time from the titration of molecular container P[6]AS (100 μM) and 1,3-propanediammonium chloride (150 μM) in the cell with MDMA (1.00 mM) in the syringe in 20 mM NaH₂PO₄buffer (pH 7.4); b) plot of the ΔH as a function of molar ratio. The solid line represents the best non-linear fit of the data to a competition binding model (Kₐ=(3.92±0.20)×10⁷ M⁻¹, ΔH=−13.3±0.1 kcal/mol, −TΔS=2.95 kcal/mol).

FIG. 70 shows a) a plot of DP vs time from the titration of molecular container P[6]AS (10 μM) in the cell with Mephedrone (100 μM) in the syringe in 20 mM NaH₂PO₄buffer (pH 7.4); b) plot of the ΔH as a function of molar ratio. The solid line represents the best non-linear fit of the data to a 1:1 binding model (Kₐ=(1.91±0.19)×10⁷ M⁻¹, ΔH=−12.6±0.11 kcal/mol, −TΔS=2.68 kcal/mol).

FIG. 71 shows a) a plot of DP vs time from the titration of molecular container P[6]AS (10 μM) in the cell with Heroin (100 μM) in the syringe in 20 mM NaH₂PO₄buffer (pH 7.4); b) plot of the ΔH as a function of molar ratio. The solid line represents the best non-linear fit of the data to a 1:1 binding model (Kₐ=(5.78±0.02)×10⁵ M⁻¹, ΔH=−11.9±0.11 kcal/mol, −TΔS=4.01 kcal/mol).

FIG. 72 shows a) a plot of DP vs time from the titration of molecular container P[6]AS (100 μM) and 17 (500 μM) with Rocuronium (1.00 mM) in 20 mM NaH₂PO₄buffer (pH 7.4); b) plot of the ΔH as a function of molar ratio. The solid line represents the best non-linear fit of the data to a competition binding model (Kₐ=(6.33±0.08)×10¹¹ M¹, ΔH=−24.9±0.177 kcal/mol, −TΔS=8.79 kcal/mol).

FIG. 73 shows a) a plot of DP vs time from the titration of molecular container P[6]AS (100 μM) and 17 (500 μM) with Vecuronium (1.00 mM) in 20 mM NaH₂PO₄buffer (pH 7.4); b) plot of the ΔH as a function of molar ratio. The solid line represents the best non-linear fit of the data to a competition binding model (Kₐ=(1.00±0.34)×10¹² M⁻¹, ΔH=−18.5±0.095 kcal/mol, −TΔS=2.10 kcal/mol).

FIG. 74 shows a) a plot of DP vs time from the titration of molecular container P[6]AS (100 μM) and 17 (150 μM) with Pancuronium (1.00 mM) in 20 mM NaH₂PO₄buffer (pH 7.4); b) plot of the ΔH as a function of molar ratio. The solid line represents the best non-linear fit of the data to a competition binding model (Kₐ=(7.35±1.23)×10¹⁰ M⁻¹, ΔH=−16.5±0.216 kcal/mol, −TΔS=1.63 kcal/mol).

FIG. 75 shows a) a plot of DP vs time from the titration of molecular container P[7]AS (10 μM) and with Cisatracurium (0.05 mM) in 20 mM NaH₂PO₄buffer (pH 7.4); b) plot of the ΔH as a function of molar ratio. The solid line represents the best non-linear fit of the data to a 1:1 binding model with n=0.5 (Kₐ=(1.52±0.12)×10⁷ M⁻¹, ΔH=−35.0±0.396 kcal/mol, −TΔS=25.2 kcal/mol).

FIG. 76 shows a) a plot of DP vs time from the titration of molecular container P[6]AS (100 μM) and propane-1,3-diaminium (150 μM) with Methamphetamine (1.00 mM) in 20 mM NaH₂PO₄buffer (pH 7.4); b) plot of the ΔH as a function of molar ratio. The solid line represents the best non-linear fit of the data to a competition binding model (Kₐ=(9.90±0.39)×10⁶ M⁻¹, ΔH=−10.4±0.040 kcal/mol, −TΔS=0.833 kcal/mol).

FIG. 77 shows a) a plot of DP vs time from the titration of molecular container P[6]AS (100 μM) and propane-1,3-diaminium (1.00 mM) with Fentanyl (1.00 mM) in 20 mM NaH₂PO₄buffer (pH 7.4); b) plot of the ΔH as a function of molar ratio. The solid line represents the best non-linear fit of the data to a competition binding model (Kₐ=(1.02±0.03)×10⁸M⁻¹, ΔH=−15.0±0.052 kcal/mol, −TΔS=4.02 kcal/mol).

FIG. 78 shows a) a plot of DP vs time from the titration of molecular container P[6]AS (100 μM) and with Cocaine (1.00 mM) in 20 mM NaH₂PO₄buffer (pH 7.4); b) plot of the ΔH as a function of molar ratio. The solid line represents the best non-linear fit of the data to a 1:1 binding model (Kₐ=(1.92±0.06)×10⁶ M⁻¹, ΔH=−15.6±0.047 kcal/mol, −TΔS=7.07 kcal/mol).

FIG. 79 shows a) a plot of DP vs time from the titration of molecular container P[6]AS (100 μM) and with Ketamine (1.00 mM) in 20 mM NaH₂PO₄buffer (pH 7.4); b) plot of the ΔH as a function of molar ratio. The solid line represents the best non-linear fit of the data to a 1:1 binding model (Kₐ=(1.52±0.25)×10⁵ M⁻¹, ΔH=−22.0±1.02 kcal/mol, −TΔS=14.9 kcal/mol).

FIG. 80 shows a) a plot of DP vs time from the titration of molecular container P[6]AS (100 μM) and propane-1,3-diaminium (150 μM) with Phencyclidine (1.00 mM) in 20 mM NaH₂PO₄buffer (pH 7.4); b) plot of the ΔH as a function of molar ratio. The solid line represents the best non-linear fit of the data to a competition binding model (Kₐ=(5.85±0.47)×10⁷ M⁻¹, ΔH=−12.4±0.076 kcal/mol, −TΔS=1.84 kcal/mol).

FIG. 81 shows a) a plot of DP vs time from the titration of molecular container P[6]AS (100 μM) and with Morphine (1.00 mM) in 20 mM NaH₂PO₄buffer (pH 7.4); b) plot of the ΔH as a function of molar ratio. The solid line represents the best non-linear fit of the data to a 1:1 binding model (Kₐ=(1.36±0.07)×10⁶ M⁻¹, ΔH=−12.9±0.073 kcal/mol, −TΔS=4.49 kcal/mol).

FIG. 82 shows a) a plot of DP vs time from the titration of molecular container P[6]AS (100 μM) and with Hydromorphone (1.00 mM) in 20 mM NaH₂PO₄buffer (pH 7.4); b) plot of the ΔH as a function of molar ratio. The solid line represents the best non-linear fit of the data to a 1:1 binding model (Kₐ=(1.31±0.04)×10⁶ M⁻¹, ΔH=−11.9±0.042 kcal/mol, −TΔS=3.55 kcal/mol).

FIG. 83 shows a) a plot of DP vs time from the titration of molecular container P[6]AS (100 μM) and with Oxycodone (1.00 mM) in 20 mM NaH₂PO₄buffer (pH 7.4); b) plot of the ΔH as a function of molar ratio. The solid line represents the best non-linear fit of the data to a 1:1 binding model (Kₐ=(9.52±0.36)×10⁴ M⁻¹, ΔH=−8.62±0.097 kcal/mol, −TΔS=1.83 kcal/mol).

FIG. 84 shows P6AS does not inhibit the hERG channel. The hERG assay was conducted using HEK-293 stably transfected with hERG cDNA in an automated QPatch HTX patch clamp study. Plot of mean hERG ion channel inhibition (%, n=3-4) versus log concentration for E-4031 (⋅) and P6AS (o).

FIG. 85 shows bacterial cytotoxicity assay conducted for P6AS (100 μM) toward the four different tester strains (TA98, TA100, TA1535, TA537).

FIG. 86 shows in vivo reversal of fentanyl induced hyperlocomotion by P6AS. Average locomotion counts for male Swiss Webster mice (n=9; avg weight (g)±SD: 34.44±2.24) are plotted as a function of treatment. All mice underwent an initial habituation to determine baseline locomotion levels before treatment. Following this baseline measure, treatment order was counterbalanced across days, and mice only received one treatment per 20 day. Over six consecutive days of testing mice each received a single treatment of PBS (PBS; 0.2 mL infused), P6AS only (P6AS; 4 mM; 0.178 mL infused), fentanyl only (Fentanyl; 0.1 mg/kg; 0.022 mL infused), a premixed solution of P6AS and fentanyl (Premix; P6AS:Fentanyl (≈68.34:1 molar ratio); 0.2 mL infused), P6AS followed by fentanyl administered 30 s (seconds) later (30s Blocking; 0.178 mL P6AS, 0.022 mL Fentanyl infused), and fentanyl followed by P6AS administered 30 s later (30 s Reversal; 0.022 mL Fentanyl, 0.178 mL P6AS infused). Bars represent average locomotion counts. Error bars represent the standard error of the mean (SEM). Dots represent counts for each mouse (n=9). Presented p-values are only for significant (p<0.05) Tukey-corrected post-hoc comparisons.

FIG. 87 shows in vivo reversal of fentanyl-induced hyperlocomotion by P6AS following 5-minute inter-injection interval. Average locomotion counts for male Swiss Webster mice (n=9) are plotted as a function of treatment. Mice receive either fentanyl (0.1 mg/mL; 0.022 mL infused) followed by PBS (0.01 M; 0.178 mL infused) or P6AS (4 mM; 0.178 mL infused) administered 5 minutes apart before being placed into the behavioral box. Bars represent average locomotion counts. Error bars represent the standard error of the mean (SEM). Dots represent counts for each mouse (n=9). Data analyzed using a paired t-test.

FIG. 88 shows in vivo reversal of fentanyl-induced hyperlocomotion by P6AS following 15-minute inter-injection interval. Average locomotion counts for male Swiss Webster mice (n=9) are plotted as a function of treatment. Mice receive either fentanyl (0.1 mg/mL; 0.022 mL infused) followed by PBS (0.01 M; 0.178 mL infused) or P6AS (4 mM; 0.178 mL infused) administered 15 minutes apart before being placed into the behavioral box. Bars represent average locomotion counts. Error bars represent the standard error of the mean (SEM). Dots represent counts for each mouse (n=9). Data analyzed using a paired t-test.

FIG. 89 shows in vivo reversal of fentanyl induced hyperlocomotion by 0.5 mM, 1.5 mM P6AS, 4.37 mM Naloxone, 1.507 mM TetM1. Average locomotion counts for male Swiss Webster mice (n=11; avg weight (g)±SD: 35.27±1.90) are plotted as a function of treatment. Treatment order was counterbalanced across days, and mice only received one treatment per day. Mice underwent 15 minute reversals where either a single 0.022 mL infusion of PBS or 0.1591 mg/mL fentanyl was followed by a 0.178 mL infusion of a candidate countermeasure. The possible six treatments included PBS followed by PBS, fentanyl followed by 1.5 mM P6AS, fentanyl followed by 0.5 mM P6AS, fentanyl followed by 4.37 mM naloxone, fentanyl followed by 1.507 mM TetM1, or fentanyl followed by PBS. Bars represent average locomotion counts. Error bars represent the standard error of the mean (SEM). Dots represent counts for each mouse (n=11). Presented p-values are only for significant (p<0.05) Tukey-corrected post-hoc comparisons. The concentrations of compounds used for the injections were selected so that the doses were as follows: P6AS (1.5 mM)=15 mg/kg=7.66 μmol/kg; P6AS (0.5 mM)=5 mg/kg=2.55 μmol/kg; TetM1 (1.5 mM)=11.81 mg/kg=7.66 μmol/kg; Naloxone (4.37 mM)=1 mg/kg=2.75 μmol/kg; Fentanyl=0.1 mg/kg. The molar ratio of P6AS (15 mg/kg):fentanyl is 28.6:1.

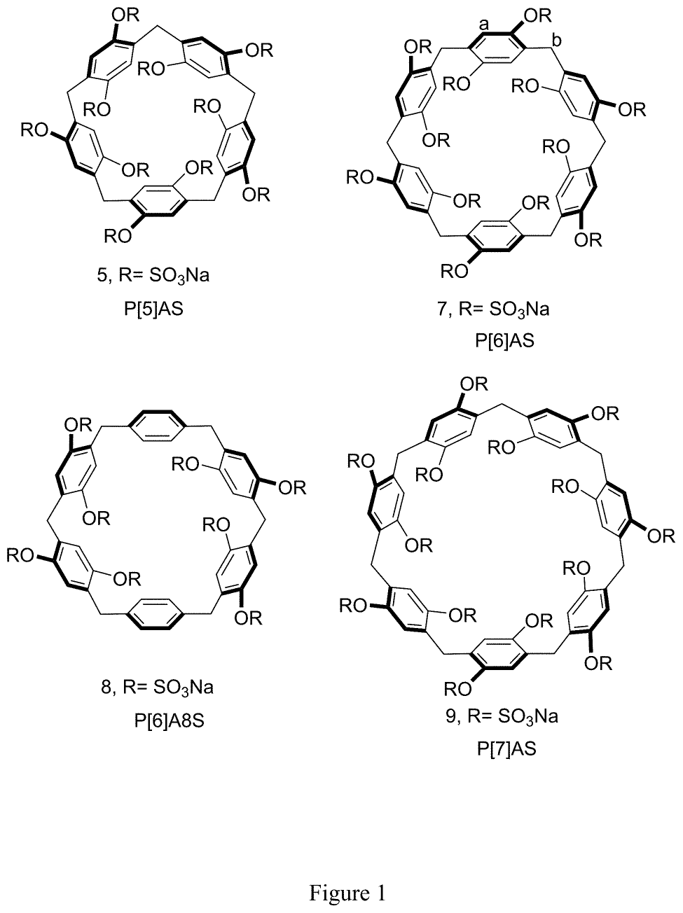

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

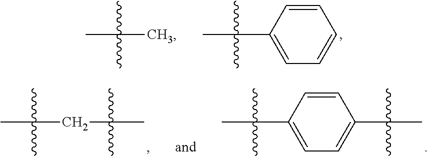

As used herein, unless otherwise indicated, the term “aryl group” refers to C₅ to C₁₈, including all integer numbers of carbons and ranges of numbers of carbons therebetween, aromatic or partially aromatic carbocyclic groups (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). An aryl group may also be referred to as an aromatic group. The aryl groups can comprise polyaryl groups such as, for example, fused ring or biaryl groups. The aryl group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof. A substituent may be or further comprise a sulfonate group or a sulfate group. Examples of aryl groups include, but are not limited to, phenyl groups, biaryl groups (e.g., biphenyl groups and the like), and fused ring groups (e.g., naphthyl groups, anthracene groups, pyrenyl groups, and the like), which may be unsubstituted or substituted.

As used herein, unless otherwise indicated, the term “heteroaryl group” refers to a C₁ to C₁₈ monocyclic, polycyclic, or bicyclic ring groups (e.g., aryl groups) comprising one or two aromatic rings containing at least one heteroatom (e.g., nitrogen, oxygen, sulfur, and the like) in the aromatic ring(s), including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, and C₁₈). The heteroaryl groups may be substituted or unsubstituted. Examples of heteroaryl groups include, but are not limited to, benzofuranyl groups, thienyl groups, furyl groups, pyridyl groups, pyrimidyl groups, oxazolyl groups, quinolyl groups, thiophenyl groups, isoquinolyl groups, indolyl groups, triazinyl groups, triazolyl groups, isothiazolyl groups, isoxazolyl groups, imidazolyl groups, benzothiazolyl groups, pyrazinyl groups, pyrimidinyl groups, thiazolyl groups, and thiadiazolyl groups, and the like. Examples of substituents include, but are not limited to, halogens (—F, —Cl, —Br, and —I), aliphatic groups (e.g., alkyl groups, alkenyl groups, alkynyl groups, and the like), aryl groups, alkoxide groups, amine groups, carboxylate groups, carboxylic acids, ether groups, alcohol groups, alkyne groups (e.g., acetylenyl groups and the like), and the like, and combinations thereof.

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁ to C₄₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkene groups, and alkyne groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof.

As used herein, unless otherwise indicated, the term “alkyl group” refers to branched or unbranched saturated hydrocarbon groups. Examples of alkyl groups include, but are not limited to, methyl groups, ethyl groups, n- and isopropyl groups, n-, iso-, sec-, and tert-butyl groups, and the like. For example, the alkyl group can be a C₁ to C₁₂, including all integer numbers of carbons and ranges of numbers of carbons therebetween (e.g., C₁, C₂, C₃, C₄, C₅, C₆, C₇, C₈, C₉, C₁₀, C₁₁, and C₁₂). The alkyl group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups (—OR, where R is an alkyl group), carboxylate groups, carboxylic acid groups, ether groups, ester groups, amide groups, thioether groups, thioester groups, and the like, and combinations thereof.

The present disclosure provides sulfated pillararenes. The present disclosure also provides method making sulfated pillararenes and uses thereof.

In this disclosure, it was shown that, for example, positioning the anionic solubilizing groups at the rim of the pillararene cavity significantly enhances their binding affinity toward cationic targets in water and thereby enhances their abilities as sequestering agents for a variety of applications.

In an aspect, the present disclosure provides compounds. The compounds are sulfated pillararenes. A sulfated pillararene comprises a macrocycle core comprising a plurality of aryl groups, where adjacent aryl groups are covalently connected (e.g., linked) via alkyl linking groups (e.g., —CH₂— groups). The alkyl linking groups are para on the aryl groups (e.g., 1,4-phenyl linkages). The linkages may be on different phenyl rings of an aryl group and correspond to a para linkage if the different phenyl rings were superimposed. In various examples, one or more or all of the adjacent aryl group(s) are not covalently connected by alkyl linking groups at meta positions on the aryl groups (e.g., 1,3-phenyl linkages (in the case where the linkages are on different phenyl rings or an aryl group the linkages do not correspond to a meta linkage if the different phenyl rings were superimposed)). Non-limiting examples of sulfated pillararenes are provided herein. Non-limiting examples of methods of making sulfated pillararenes are provided herein.

In various examples, a sulfated pillararene has the following structure:

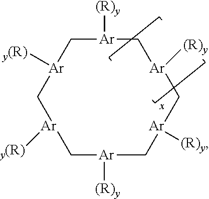

where Ar is an aryl group attached (e.g., covalently bonded) in a para orientation to the adjacent methylene groups (e.g., 1,4-phenyl group linkage), which may be a part of a larger aryl group; each R is independently chosen from —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)), —OS(O)₂OH, non-sulfate anionic groups (such as, for example, sulfonate (and corresponding acid) groups (e.g., —O(CH₂)ₘS(O)₂O⁻M⁺(where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘS(O)₂OH, where m is 1 to 8 (e.g., 1, 2, 3, 4, 5, 6, 7, 8), —C₆H₅S(O)₂OH, and the like and such groups where the terminal O is removed), carboxylate (and corresponding acid) groups (e.g., —O(CH₂)ₘC(O)O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘC(O)OH, where n is 1 to 8 (e.g., 1, 2, 3, 4, 5, 6, 7, 8), and the like, such as for example, —OCH₂CO₂⁻M⁺/—OCH₂CO₂H groups and the like and such groups where the terminal O is removed), phosphonate (and corresponding acid) groups (e.g., —O(CH₂)ₘP(O)(OH)₂M⁺(where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘP(O)(OH)₂, where m is 1 to 8 (e.g., 1, 2, 3, 4, 5, 6, 7, 8), and the like, such as for example, —O(CH₂)₂P(O)(OH)₂ and the like and such groups where the terminal O is removed), phosphate groups —OP(O)(OH)₂, and the like), substituted or unsubstituted aryl groups, substituted or unsubstituted heteroaryl groups, substituted or unsubstituted aliphatic groups, O-alkyl groups (comprising an alkyl group), azide groups, —H, substituted or unsubstituted alkyl groups, halogens (e.g., —Br, —F, —I, —Cl), amide groups, cyano groups, substituted or unsubstituted sulfur-containing aliphatic groups (e.g., —S-alkyl and poly thioethers, and the like), nitro groups, amino groups, substituted or unsubstituted nitrogen-containing aliphatic groups (e.g., polyamines, aliphatic groups comprising secondary and/or tertiary amines, and the like), substituted or unsubstituted polyethylene glycol groups, polyether groups, O-aryl groups (e.g., aryloxy groups), ester groups, carbamate groups, imine groups, aldehyde groups, —SO₃H groups, —SO₃Na groups, —OSO₂F groups, —OSO₂CF₃ groups, —OSO₂OR′″ groups (where R′″ are substituted or unsubstituted aryl groups or substituted or unsubstituted alkyl groups), and the like, and combinations thereof, x is 0, 1, 2, or 3; and y is independently at each occurrence 0, 1, 2, 3, or 4, with the proviso that at least one y is 1 and at least one R group is —OS(O)₂O⁻M⁺(where M⁺ is Na⁺, K⁺) or —OS(O)₂OH, or a salt, a partial salt, a hydrate, a polymorph, a stereoisomer, conformational isomer, or a mixture thereof. The R group(s) may be at any position(s) on an aryl group. In the case of an aryl group with multiple R groups, the individual R groups may be at any combination of positions of the aryl group. In various examples, all of the aryl groups comprise an R group that is independently —OS(O)₂O⁻M⁺ (where M⁺ is Na⁺, K⁺, Ca², Mg²⁺, Zn²⁺, H₄N⁺, Et₃NH⁺, Me₄N⁺, (HOCH₂CH₂)₃NH⁺, or a cationic form of ethylenediamine, piperazine, and trishydroxymethyl aminomethane (TRIS)) or —OS(O)₂OH. In various examples, at least one aryl group does not comprise an R group that is —OS(O)₂O⁻M⁺(where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) or —OS(O)₂OH. In various embodiments, the aryl groups may be further substituted with various substitutents, such as, for example, —H, alkyl groups, aliphatic groups, polyethylene glycol groups, or the like, or a combination thereof.

In certain embodiments, M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS).

In certain embodiments, M⁺ is Na⁺, K⁺, and H₄N⁺. In certain embodiments, M⁺ is Na⁺.

A sulfated pillararene can comprise various aryl groups. The aryl groups may all be the same or at least two of the aryl groups are different. Non-limiting examples of aryl groups are independently at each occurrence chosen from phenyl groups, fused-ring groups (e.g., naphthyl groups, anthracenyl groups, phenanthrenyl groups, tetracenyl groups, pentacenyl groups, and the like), biaryl groups (e.g., biphenyl groups and the like), terphenyl groups, and the like, and combinations thereof. For avoidance of doubt, a phenyl group, when it is not part of a larger aryl group, unless otherwise described, is a C₆H₄ group. A phenyl group may be referred to as a phenylene group.

Adjacent aryl groups can be linked by various linkages. The linkages are para-linked phenyl group linkages. In various examples, at least a portion or all of the linkages are 1,4-phenyl group linkage(s). Non-limiting examples of para-linked phenyl group linkages include:

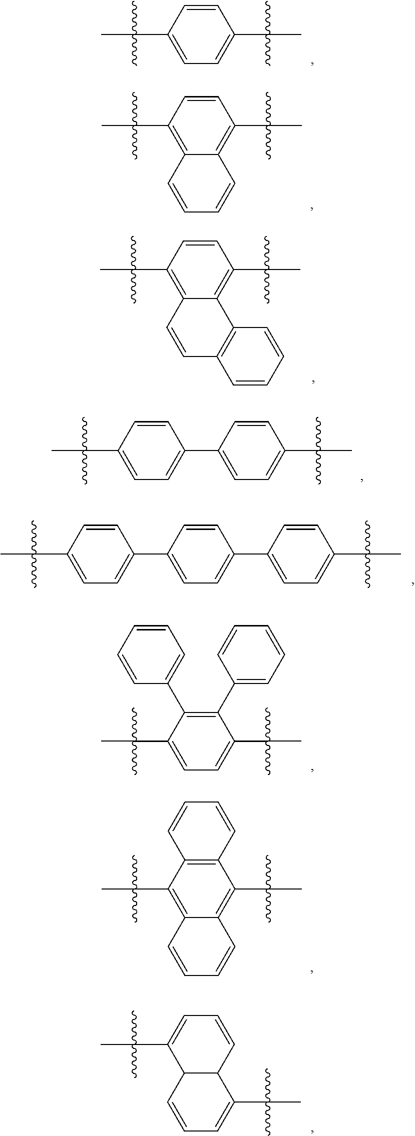

and combinations thereof. These are illustrative examples. Other para-linked phenyl group linkages are within the scope of this disclosure. In various examples, the linkage is not a meta linkage.

An aryl group may comprise one or more phenyl group(s). In various non-limiting examples, at least two, at least three, or at least 4, or all of the one or more phenyl group(s) of one or more of the aryl group(s) comprising the cyclic core of the compound have at least 1 or at least 2 R groups independently chosen from —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) and —OS(O)₂OH. All of the aryl groups, one or more or all of which may be phenyl group(s), may comprise a sulfate group —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) or —OS(O)₂OH. In various examples, at least one aryl group, which may be a phenyl group, does not comprise a sulfate group (e.g., —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) or —OS(O)₂OH).

In various examples, a sulfated pillararene has the following structure:

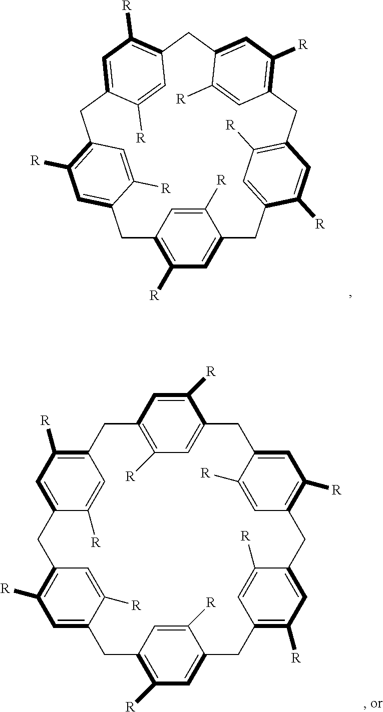

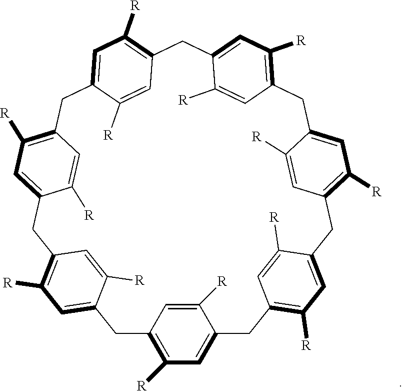

In various examples, each R is —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) and —OS(O)₂OH.

In various examples, a sulfated pillararene has the following structure:

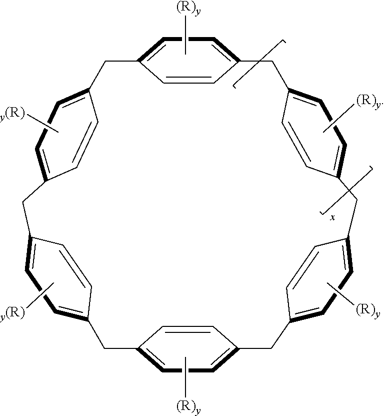

In various examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of the R groups are independently —OS(O)₂O⁻M⁺ groups (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) or —OS(O)₂OH groups.

In an aspect, the present disclosure provides compositions comprising one or more sulfated pillararene(s). Non-limiting examples of compositions are described herein.

A composition may comprise one or more sulfated pillararene(s) and one or more pharmaceutical agent(s). In various examples, a pharmaceutical agent comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

A composition may comprise one or more sulfated pillararene(s), one or more pharmaceutical carrier(s), and, optionally, one or more pharmaceutical agent(s). The compositions described herein can be with one or more pharmaceutically acceptable carrier(s). Suitable pharmaceutically acceptable carriers are known in the art. Some non-limiting examples of pharmaceutically acceptable carriers can be found in: Remington: The Science and Practice of Pharmacy (2005) 21st Edition, Philadelphia, Pa. Lippincott Williams & Wilkins. In various examples, the pharmaceutical carrier is pure water or a buffer, such as PBS buffer or the like.

Compositions comprising one or more sulfated pillararene(s) combined with one or more pharmaceutical agent(s), which may form guest-host complexes, can be prepared at any point prior to use of the composition using any suitable technique. The compound-pharmaceutical agent complexes can be formed, for example, by mixing the compound and the pharmaceutical agent in a suitable solvent. It is desirable that the compound and pharmaceutical agent be soluble in the solvent such that the compound and agent form a non-covalent complex. Any suitable solvent can be used. In certain examples, the solvent is an aqueous solution, which includes, but is not necessarily limited to, water and various buffers (e.g., PBS buffer and the like). Non-aqueous solvents could also be used (e.g., MeOH, EtOH, and other organic solvents, and combinations thereof), and then removed and the compositions if desired can be re-dissolved in an aqueous solution for administration. In general, a solution of a compound(s) can be provided at a known concentration, examples of which include but are not limited to from 0.1 to 90 mM, inclusive and including all integers to the tenth decimal place there between, and a pharmaceutical agent for which enhanced solubility is desired is added to the solution. The agent(s) can be provided, for example, in a solid form. The combination can be shaken or stirred for a period of time and the amount of pharmaceutical agent that is dissolved is monitored. If all added agent goes into solution, more agent can be added until some detectable portion of it remains undissolved (e.g., a solid). The soluble compound-agent complex can then be isolated and analyzed by any suitable technique, such by recovering a centrifuged portion and analyzing it by NMR, to determine the concentration of pharmaceutical agent in solution. In various examples, a compound is provided in a composition comprising the drug at a ratio of at least 1 to 1 as pertains to the compound-agent stoichiometry (e.g., pillararene to drug ratio). In various examples, the pillararene (e.g., pillararene sulfate) to drug ratio is 100:1 to 1:5, including all ratio values and ranges therebetween (e.g., 100:1, 5:1, 1:2, 1:3, 1:4, or 1:5).

Compositions may be prepared at a patient's bedside or by a pharmaceutical manufacture. In the latter case, the compositions can be provided in any suitable container, such as, for example, a sealed sterile vial, ampoule, or the like, and may be further packaged (the combination of which may be referred to as a kit) to include instruction documents for use by a pharmacist, physician, other health care provider, or the like. The compositions can be provided as a liquid, or as a lyophilized or powder form that can be reconstituted if necessary when ready for use. In particular, the compositions can be provided in combination with any suitable delivery form or vehicle, examples of which include but are not limited to liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agent(s).

Compositions of the present disclosure may comprise more than one pharmaceutical agent. Likewise, the compositions can comprise distinct host-guest complexes. For example, a first composition comprising one or more sulfated pillararene(s) and a first pharmaceutical agent can be separately prepared from a composition which comprises the same compound and a second pharmaceutical agent, and such preparations can be mixed to provide a two-pronged (or more) approach to achieving the desired prophylaxis or therapy in an individual. Further, compositions can be prepared using mixed preparations of any of the sulfated pillararene compounds disclosed herein.

A solid substrate may comprise one or more sulfated pillararene(s) disposed on (e.g., chemically bonded to) at least a portion of a surface of the substrate. At least a portion or all of the sulfated pillararenes may be chemically bonded to at least a portion of a surface by covalent bonds, non-covalent bonds, or a combination thereof. Methods of conjugating sulfated pillararenes to solid surfaces are known in the art. In various examples, sulfated pillararenes are conjugated to a surface by covalent bond- and/or non-covalent bond forming reactions including, but not limited to, amide bond formation, azide alkyne cycloaddition, gold thiol interactions, silicon alcohol condensations, and the like, and combinations thereof.

A solid substrate may comprise (or be) various materials. In various non-limiting examples, a solid substrate comprises or is silica (such as, for example, silica particles), polymer beads, polymer resins (such as, for example, polystyrene, poly NIPAM, polyacrylic acid, metal nanoparticles (e.g. gold nanoparticles, silver nanoparticles, magnetic nanoparticles), a metal (such as, for example, gold and the like), or the like, or a combination thereof.

In an aspect, the present disclosure provides uses of sulfated pillararenes. Non-limiting examples of uses of sulfated pillararenes are provided herein, for example, non-limiting examples of uses of sulfated pillararenes are described in the Statements and Examples.

Sulfated pillararenes can be used to sequester various materials, which may be chemical compounds. In various non-limiting examples, one or more sulfated pillararene(s) is/are used to sequester one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like), drugs of abuse (e.g., methamphetamine, cocaine, fentanyl, carfentanil, PCP, MDMA, heroin, and the like), and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescein, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g. tabun, sarin, soman, cyclosarin, 2-(dimethylamino)ethyl N,N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g. mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g. 4-(methylnitrosoamino)-1-(3-pyridyl)-1-butanone); or the like, or a combination thereof.

A material, which may be a chemical compound, may comprise one or more cationic group. In various examples, a material, which may be a chemical compound, comprises one or more positively charged nitrogen atom(s) (e.g., ammonium ions, primary ammonium ions, secondary ammonium ions, tertiary ammonium ions, quaternary ammonium ions, or a combination thereof, where the non-hydrogen group(s) on the ammonium are chosen from aliphatic groups, alkyl groups, aryl groups, and combinations thereof).

In various examples, a method for sequestering one or more neuromuscular blocking agent(s), one or more anesthesia agent(s), one or more pharmaceutical agent(s), one or more pesticide(s), one or more dyestuff(s), one or more malodorous compound(s), one or more chemical warfare agent(s), one or more hallucinogen(s), one or more toxin(s), one or more metabolite(s) or the like, or a combination thereof comprises contacting the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof with one or more sulfated pillararene(s) and/or one or more composition(s), where the neuromuscular blocking agent(s), the anesthesia agent(s), the pharmaceutical agent(s), the pesticide(s), the dyestuff(s), the malodorous compound(s), the chemical warfare agent(s), or a combination thereof are sequestered by the one or more sulfated pillararene(s) and/or one or more composition(s).

The neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof may be present in an aqueous sample, in a solid sample (such as, for example, a soil sample), in a gas sample, or the like. An aqueous sample may be derived (e.g., via extraction or other methods to isolate the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof from the solid sample). The aqueous sample may be a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.

A composition may comprise one or more pharmaceutically active agent(s). In various non-limiting examples, at least a portion (or all) of the one or more compound(s) have a pharmaceutically active agent(s) disposed in the cavity of the one or more compound(s). Without intending to be bound by any particular theory, it is considered that a complex (which may be referred to as a guest-host complex) is formed from (e.g., one or more interaction(s) between (e.g., one or more non-covalent interactions, such as, for example, one or more non-covalent bond(s), is formed between) the compound(s), which may be referred to as hosts, and the neuromuscular blocking agent(s), the anesthesia agent(s), the pharmaceutical agent(s), which may be pharmaceutical agent(s) with undesirable (e.g., low) water solubility, the pesticide(s), the dyestuff(s), the malodorous compound(s), the chemical warfare agent(s), the hallucinogen(s), the toxin(s), the metabolite(s), or a combination thereof, which may be referred to a guest or guests. A guest-host complex can therefore be considered to be an organized chemical entity resulting from the association of the pharmaceutical agent(s) (guest(s)) and the host held together, for example, by non-covalent intermolecular forces.

A composition can comprise various pharmaceutically active agents. Non-limiting examples of pharmaceutical agents include drugs. The pharmaceutically active agent(s) may have various aqueous solubility. A pharmaceutically active agent may have hydrophobic, hydrophilic, or amphiphilic character.

The complexes may be removed from the aqueous sample, the solid sample, the gas sample, or the like. In various examples, the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof are removed from the aqueous sample, the solid sample, the gas sample, or the like using a solid surface with one or more sulfated pillararene(s) disposed thereon.

Sulfated pillararenes can be used to sequester various materials in an individual. In various non-limiting examples, the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in an individual and the contacting comprises administration of the one or more compound(s) and/or one or more composition(s) to the individual.

Sulfated pillararenes can be used to reverse drug-induced neuromuscular block and/or anesthesia and/or the effects of one or more drug(s), which may be drugs of abuse in an individual.

In various non-limiting examples, a method for reversing drug-induced neuromuscular block and/or anesthesia and/or the effects of one or more pharmaceutical agent(s) (e.g., one or more drug(s) of abuse) in or on an individual comprising administering to an individual in need of reversal of neuromuscular block and/or reversal of anesthesia and/or reversal of the effects of the one or more pharmaceutical agent(s) (e.g., one or more drug(s) of abuse), one or more sulfated pillararenes, and/or one or more composition(s). The individual may be in need of reversal of drug-induced neuromuscular block. The individual may be in need of reversal of anesthesia. The individual may be in need of reversal of drug-induced neuromuscular block and anesthesia. The individual may be in need of reversal of the effects of one or more pharmaceutical agent(s), such as, for example, one or more drug(s), which may be drug(s) of abuse. The individual may have been exposed to the drug(s) of abuse (e.g., carfentanil and the like) in a terrorist attack.

The sulfated pillararene compounds may be used as containers to solubilize chemical compounds. Improvement of solubility for compounds in, for example, aqueous solutions, is desirable for studying drug compounds and for improvement of drug bioavailability for purposes such as, for example, therapeutic and/or prophylactic purposes. For example, the sulfated pillararenes are be used to enhance the stability (e.g., decrease degradation, increase shelf life, and the like) of drugs in water, the solid state, or both.

In certain examples, the sulfated pillararene compounds can be used to rescue promising drug candidates, which have undesirable solubility and bioavailability, and thus alleviate the attrition in the drug development process for anti-cancer agents and agents intended to treat other diseases. The containers may be used for targeted delivery of drugs to particular cell types, such as, for example, tumor cells and the like, to increase the effectiveness of existing drugs, reduce their toxic side effect(s), or both.

In various examples, a composition comprises one or more sulfated pillararene(s) and one or more pharmaceutical agent(s). Such compositions may be provided as pharmaceutical preparations as described herein.

It is important to emphasize that the pharmaceutical agent(s) that can be included in compositions comprising one or more sulfated pillararene(s) and one or more pharmaceutical agent(s) is not particularly limited. In certain examples, the pharmaceutical agent(s) combined with one or more sulfated pillararene(s) is/are a pharmaceutical agent or agents that is/are poorly water-soluble. In certain other examples, the pharmaceutical agent(s) combined with one or more sulfated pillararene(s) is/are a pharmaceutical agent or agents that is/are water soluble.

Solubility of any particular pharmaceutical agent can be determined, if desired, using any of a variety techniques that are well known to those skilled in the art. Solubility can be ascertained if desired at any pH, such as a physiological pH, and/or at any desired temperature. Suitable temperatures include, but are not necessarily limited to, from 4° C. to 70° C., inclusive, and including all integer ° C. values therebetween.

In connection with poorly soluble or low solubility pharmaceutical agents suitable for use in the present disclosure, in various examples, such agents are considered to be those which have a solubility of less than 100 μM in water or an aqueous buffer.

In various other examples, poorly soluble pharmaceutical agents are considered to include compounds, which are Biopharmaceutics Classification System (BCS) class 2 or class 4 drugs. The BCS is well known to those skilled in the art and is based on the aqueous solubility of drugs reported in readily available reference literature, and for drugs that are administered orally it includes a correlation of human intestinal membrane permeability. (See, for example, Takagi et al., (2006) Molecular Pharmaceutics, Vol. 3, No. 6, pp. 631-643.) The skilled artisan will therefore readily be able to recognize a drug as a member of BCS class 2 or class 4 from published literature, or can test a drug with an unknown BCS or other solubility value to determine whether it has properties consistent with either of those classifications, or for otherwise being suitable for use in the present disclosure. In an example, solubility is determined according to the parameters set forth in this matrix:

| | Parts of solvent required for | Solubility Range |
|---|---|---|
| Solubility | 1 part of solute | (mg/mL) |
| very soluble | <1 | ≥1000 |
| freely soluble | from 1 to 10 | 100-1000 |
| soluble | from 10 to 30 | 33-100 |
| sparingly soluble | from 30 to 100 | 10-33 |
| slightly soluble | from 100 to 1000 | 1-10 |
| very slightly soluble | form 1000 to 10000 | 0.1-1 |
| practically insoluble | ≥10000 | <0.1 |

Thus, for the purposes of the present disclosure, a poorly soluble pharmaceutical agent that can be combined with one or more sulfated pillararene(s) can be any pharmaceutical agent that falls into the categories sparingly soluble, slightly soluble, very slightly soluble, and practically insoluble as set forth in the above matrix.

Again, it should be emphasized that other than being characterized as having low solubility in aqueous solution, the pharmaceutical agent with which one or more sulfated pillararene(s), which a compound can be combined is not limited. In this regard, at least one utility of the present disclosure is combination of one or more of a wide variety of distinct pharmaceutical agents with one or more sulfated pillararene(s), and as a consequence of combining these compounds with the pharmaceutical agent(s), solubility of the agent(s) is/are increased. In various examples, types of pharmaceutical agents suitable for solubilization include, but are not limited to, mitotic inhibitors (e.g., taxol, a mitotic inhibitor used in cancer chemotherapy, and the like); nitrogen mustard alkylating agents (e.g., Melphalan, trade name Alkeran used for chemotherapy, and the like); benzimidazoles (e.g., Albendazole, marketed as Albenza, Eskazole, Zentel and Andazol, for treatment of a variety of worm infestations, and the like); antagonists of the estrogen receptor in breast tissue which is used to treat breast cancers (e.g., Tamoxifen, which is an estrogen receptor antagonist when metabolized to its active form of hydroxytamoxifen, and the like); antihistamines (e.g., Cinnarizine, marketed as Stugeron and Stunarone for control of symptoms of motion sickness, and the like); thienopyridine class antiplatelet agents (e.g., Clopidogrel, marketed as Plavix for inhibiting blood clots in coronary artery disease and for other conditions, and the like); and antiarrhythmic agents (e.g., Amiodarone, used for treatment of tachyarrhythmias, and the like). Other pharmaceutical agents not expressly listed here are also included within the scope of the disclosure. Some examples of such agents include, but are not limited to, adjuvants for use in enhancing immunological responses, analgesic agents, detectably labeled agents used for diagnostic imaging, and the like. Combinations of any of these example pharmaceutical agents may be used. Sulfated pillararenes may be combined with and improve solubility of pharmaceutical agents that are members of vastly different classes of compounds which are characterized by disparate chemical structures and biological activities.

Compositions of the present disclosure can be administered to any human or non-human animal in need of therapy or prophylaxis for one or more condition(s) for which the pharmaceutical agent is intended to provide a prophylactic of therapeutic benefit. Thus, the individual can be diagnosed with, suspected of having, or be at risk for developing any of a variety of conditions for which a reduction in severity would be desirable. Non-limiting examples of such conditions include cancer, including solid tumors, blood cancers (e.g., leukemia, lymphoma, myeloma, and the like). Specific examples of cancers include, but are not limited to, fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, heavy chain disease, and the like.

In addition to various malignancies, compounds of the present disclosure are also suitable for providing a benefit for cardiovascular related disorders, examples of which include, but are not limited to, angina, arrhythmia, atherosclerosis, cardiomyopathy, congestive heart failure, coronary artery disease, carotid artery disease, endocarditis, coronary thrombosis, myocardial infarction, hypertension, hypercholesterolemia/hyperlipidemia, mitral valve prolapse, peripheral artery disease, stroke, thrombosis, embolism, other forms of ischemic damage, and the like.

In addition, the compositions of the present disclosure can be used in connection with treating a variety of infectious diseases. It is expected that a variety of agents used to treat and/or inhibit infectious diseases caused by, for example, bacterial, protozoal, helminthic, fungal origins, viral origins, or the like can be aided by use of compositions of the present disclosure.

Various methods known to those skilled in the art can be used to introduce the compounds and/or compositions of the present disclosure to an individual. These methods include, but are not limited to, intravenous, intramuscular, intracranial, intrathecal, intradermal, subcutaneous, oral routes, and the like, and combinations thereof. The dose of the composition comprising a compound and a pharmaceutical agent will necessarily be dependent upon the needs of the individual to whom the composition is to be administered. These factors include, but are not necessarily limited to, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

The steps of the method described in the various examples disclosed herein are sufficient to carry out the methods of the present disclosure. Thus, in an example, the method consists essentially of a combination of the steps of the methods disclosed herein. In another example, the method consists of such steps.

In an aspect, the present disclosure provides articles comprising compounds of the present disclosure.

The articles may be articles of manufacture. Non-limiting examples of articles include wipes impregnated with one or more compounds of the present disclosure. For example, such a wipe is used to decontaminate a surface from any material capable of being sequestered by a compound (e.g., pillararene of the present disclosure). For example, the wipe is used to decontaminate a surface that has or was previously exposed to a toxin, abused drug, or the like, or a combination thereof.

The following Statements illustrate various embodiments of the present disclosure.

Statement 1. A compound having the following structure:

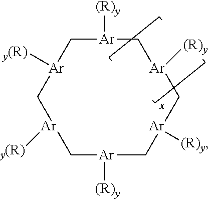

where Ar is an aryl group where adjacent aryl groups are linked by a para-linked phenyl group linkages (e.g., 1,4-phenyl group linkage(s)) (e.g., the aryl groups are attached in a para orientation to the adjacent methylene groups), which may be a part of a larger aryl group; each R is independently chosen from —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)), and —OS(O)₂OH, non-sulfate anionic groups (such as, for example, sulfonate (and corresponding acid) groups (e.g., —O(CH₂)ₘS(O)₂O⁻M⁺(where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘS(O)₂OH, where n is 1 to 8 (e.g., 1, 2, 3, 4, 5, 6, 7, 8), —C₆H₅S(O)₂OH, and the like and such groups where the terminal O is removed), carboxylate (and corresponding acid) groups (e.g., —O(CH₂)ₘC(O)O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘC(O)OH, where m is 1 to 8 (e.g., 1, 2, 3, 4, 5, 6, 7, 8), and the like, such as for example, —OCH₂CO₂⁻M⁺/-OCH₂CO₂H groups and the like and such groups where the terminal O is removed), phosphonate (and corresponding acid) groups (e.g., —O(CH₂)ₘP(O)(OH)₂M⁺(where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS))/-O(CH₂)ₘP(O)(OH)₂, where m is 1 to 8 (e.g., 1, 2 3, 4, 5, 6, 7, 8), and the like, such as for example, —O(CH₂)₂P(O)(OH)₂ and the like and such groups where the terminal O is removed), phosphate groups —OP(O)(OH)₂, and the like), substituted or unsubstituted aryl groups, substituted or unsubstituted heteroaryl groups, substituted or unsubstituted aliphatic groups, O-alkyl groups (comprising an alkyl group), polyether groups (e.g., polyethylene glycol (PEG) groups), azide groups, —H, substituted or unsubstituted alkyl groups, halogens (e.g., —Br, —F, —I, —Cl), amide groups, cyano groups, substituted or unsubstituted sulfur-containing aliphatic groups (e.g., —S-alkyl and poly thioethers, and the like), nitro groups, amino groups, substituted or unsubstituted nitrogen-containing aliphatic groups (e.g., polyamines, aliphatic groups comprising secondary and/or tertiary amines, and the like), substituted or unsubstituted polyethylene glycol groups, polyether groups, O-aryl groups (e.g., aryloxy groups), ester groups, carbamate groups, imine groups, aldehyde groups, —SO₃H groups, —SO₃Na groups, —OSO₂F groups, —OSO₂CF₃ groups, —OSO₂OR′″ groups (where R′″ are substituted or unsubstituted aryl groups or substituted or unsubstituted alkyl groups), and the like, and combinations thereof; x is 0, 1, 2, or 3; and y is independently at each occurrence 0, 1, 2, 3, or 4, with the proviso that at least one y is 1 and at least one R group is —OS(O)₂O⁻M⁺ (where M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS)) or —OS(O)₂OH, or a salt, a partial salt, a hydrate, a polymorph, a stereoisomer, conformational isomer, or a mixture thereof. The R group(s) may be at any position(s) on an aryl group. In the case of an aryl group with multiple R groups, the individual R groups may be at any combination of positions of the aryl group. In various embodiments, the aryl groups may be further substituted with various substituents.
Statement 2. A compound according to Statement 1, where the aryl groups are independently at each occurrence chosen from phenyl groups, fused-ring groups (e.g., naphthyl groups, anthracenyl groups, phenanthrenyl groups, tetracenyl groups, pentacenyl groups, and the like), biaryl groups (e.g., biphenyl groups and the like), terphenyl groups, and the like.
Statement 3. A compound according to Statements 1 or 2, where at least two, at least three, or at least 4, or all of the one or more phenyl group(s) of one or more of the aryl group(s) comprising the cyclic core of the compound have at least 1 or at least 2 R groups independently chosen from —OS(O)₂O⁻M⁺ and —OS(O)₂OH.
Statement 4. A compound according to Statement 3, where the compound has the following structure:

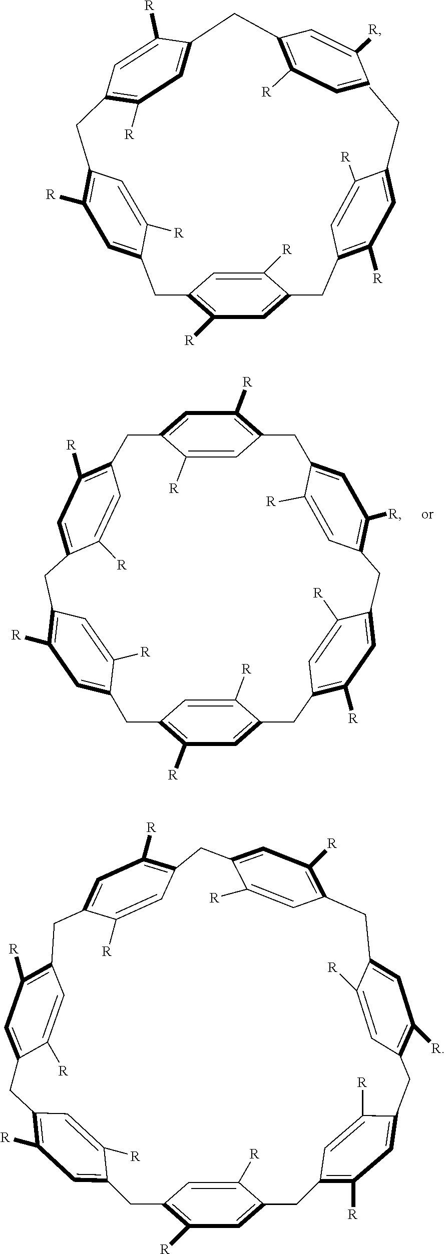

In various examples, each R is —OS(O)₂O⁻M⁺ and —OS(O)₂OH.
Statement 5. A compound according to any one of the preceding Statements, where all of the aryl groups comprise an R group that is independently —OS(O)₂O⁻M⁺ or —OS(O)₂OH.
Statement 6. A compound according to any one of Statements 1-3, where at least one aryl group does not comprise an R group that is —OS(O)₂O⁻M⁺ or —OS(O)₂OH.
Statement 7. A compound according to Statement 1, where the compound has the following structure:

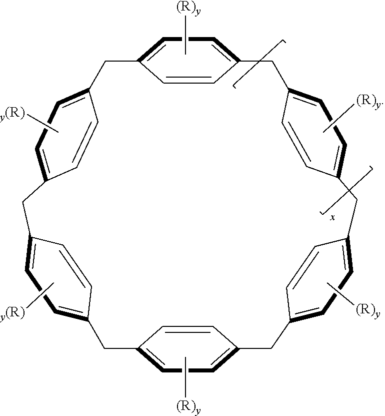

Statement 8. A compound according to Statement 7, where 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of the R groups are independently —OS(O)₂O⁻M⁺ groups or —OS(O)₂OH groups.
Statement 9. A compound according to Statements 7 or 8, where each phenyl group comprising the cyclic core of the compound has at least 1 or at least 2 R groups independently chosen from —OS(O)₂O⁻M⁺ and —OS(O)₂OH.
Statement 10. A compound according to any one of Statements 7-9, where at least one phenyl group does not comprise an R group that is —OS(O)₂O⁻M⁺ or —OS(O)₂OH.
Statement 11. A composition comprising one of more compound(s) according to any one of the preceding Statements.
Statement 12. A composition according to Statement 11, further comprising a pharmaceutical carrier.
Statement 13. A composition according to Statement 11, where the one or more compound(s) are disposed (e.g., chemically bonded) to at least a portion of a solid substrate.
Statement 14. A composition according to Statement 13, where the solid substrate comprises (or is) silica (such as, for example, silica particles), polymer beads, polymer resins (such as, for example, polystyrene, poly NIPAM, polyacrylic acid), metal nanoparticles (e.g. gold nanoparticles, silver nanoparticles, magnetic nanoparticles), a metal (such as, for example, gold and the like), or the like, or a combination thereof.
Statement 15. A composition according to any one Statements 11-14, where at least a portion (or all) of the one or more compound(s) have a pharmaceutically active agent(s) disposed in the cavity of the one or more compound(s) (e.g., non-covalently complexed to the compound(s)).
Statement 16. A method for sequestering: one or more neuromuscular blocking agent(s) (such as, for example, rocuronium, tubocurarine, atracurium, (cis)atracurium besylate, mivacurium, gallamine, pancuronium, vecuronium, and rapacuronium, and the like); one or more anesthesia agent(s) (such as, for example, N-methyl D-aspartate (NMDA) receptor antagonists (e.g., ketamine and the like), short-acting anesthetic agents (e.g., etomidate and the like), and the like); one or more pharmaceutical agent(s) (such as, for example, a drug (e.g., anticoagulants, such as, for example, hexadimethrine and the like), drugs of abuse (e.g., methamphetamine, cocaine, fentanyl, carfentanil, PCP, MDMA, heroin, and the like), and the like); one or more pesticide(s) (such as, for example, paraquat, diquat, organochlorines (e.g., DDT, aldrin, and the like), neonicotinoids (e.g., permethrin and the like), organophosphates (e.g., malathion, glyphosate, and the like), pyrethroids, triazines (e.g., atrazine and the like), and the like); one or more dyestuff(s) (such as, for example, methylene blue, nile red, crystal violet, thioflavin T, thiazole orange, proflavin, acridine orange, methylene violet, azure A, neutral red, cyanines, Direct orange 26, disperse dyes (e.g., disperse yellow 3, disperse blue 27, and the like), coumarins, congo red, and the like); one or more malodorous compound(s) (such as, for example, low molecular weight thiols (e.g., C₁-C₄ thiols), low molecular weight amines (e.g., triethylamine, putrescein, cadaverine, and the like), and the like); or one or more chemical warfare agent(s) (such as for example, nitrogen and sulfur mustards (e.g., bis(2-chloroethyl)ethylamine, bis(2-chloroethyl)methylamine, tris(2-chloroethyl)amine, bis(2-chloroethyl) sulfide, bis(2-chloroethylthioethyl) ether, and the like), nerve agents (such as, for example, those from the G, GV, and V series of nerve agents (e.g. tabun, sarin, soman, cyclosarin, 2-(dimethylamino)ethyl N,N-dimethylphosphoramidofluoridate (GV), novichok agents, VE, VG, VM, VX, and the like), and the like); one or more hallucinogen(s) (e.g., ergolines, lysergic acid diethylamide (LSD), psilocybin, tryptamines, dimethyltryptamine (DMT), phenethylamines, mescaline, ayahuasca, dextromethorphan, and the like); one or more toxin(s) (e.g., dioxins, perfluoralkylsulfonates (PFAS), perfluorooctanoic acid (PFOA), decabromobiphenyl ether (DECA), heavy metals (e.g. mercury), muscarine, tyramine, strychnine, tetrodotoxin, saxitoxin and the like, cholesterol, deoxycholic acid, N-Methyl-4-phenyl-1,2,3,6-tetrahydropyridine, phenylalanine, tyrosine, arginine, histamine); one or more metabolite(s) (e.g., toxic metabolites, such as, for example, N-methyl-4-phenylpyridine, spermine, spermidine, N-nitroso compounds e.g. 4-(methylnitrosoamino)-1-(3-pyridyl)-1-butanone); or the like, or a combination thereof are sequestered by the one or more compound(s) according to any one of Statements 1-10 and/or one or more composition(s) according to any one of Statements 11-14.
Statement 17. A method according to Statement 16, where the neuromuscular blocking agent(s), the anesthesia agent(s), the pharmaceutical agent(s), the pesticide(s), the dyestuff(s), the malodorous compound(s), the chemical warfare agent(s), the hallucinogen(s), the toxin(s), the metabolite(s), or a combination thereof is present in an aqueous sample, in a solid sample (such as, for example, a soil sample), in a gas sample, on a solid surface, or the like.
Statement 18. A method according to Statement 17, where the aqueous sample is a wastewater sample (e.g., a municipal wastewater sample, industrial wastewater sample, and the like), an industrial water sample (e.g., water used to make a commercial product, such as, for example, a reagent, a solvent, or the like), a municipal water sample, or the like.
Statement 19. A method according to any one of Statements 16-18, where a complex is formed from (e.g., one or more interaction(s) between (e.g., one or more non-covalent bond(s) is formed between) the compound(s) and the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof.
Statement 20. A method according to any one of Statements 16-19, where the complex is removed from the aqueous sample, the solid sample, the gas sample, or the like.
Statement 21. A method according to Statement 16, where the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof is present in and/or on an individual and the contacting comprises administration of the one or more compound(s) and/or one or more composition(s) to the individual.
Statement 22. A method according to Statement 21, where the individual is a human or a non-human mammal.
Statement 23. A method for reversing drug-induced neuromuscular block and/or anesthesia and/or the effects of one or more pharmaceutical agent(s) (e.g., one or more drug(s) of abuse) in an individual comprising administering to an individual in need of reversal of neuromuscular block and/or reversal of anesthesia and/or reversal of the effects of one or more pharmaceutical agent(s) (e.g., one or more drug(s) of abuse) one or more compound(s) according to any one of Statements 1-10 and/or one or more composition(s) according to any one of Statements 11-14.
Statement 24. A method according to Statement 23, where the individual is in need of reversal of drug-induced neuromuscular block.
Statement 25. A method according to Statement 23, where the individual is in need of reversal of anesthesia.
Statement 26. A method according to Statement 23, where the individual is in need of reversal of drug-induced neuromuscular block and anesthesia.
Statement 27. A method according to Statement 23, where the individual is in need of reversal of the effects of one or more pharmaceutical agent(s) are chosen from one or more drug(s) of abuse, one or more pesticide(s), one or more chemical warfare agent(s), one or more nerve agent(s), one or more hallucinogen(s), one or more toxin(s), and/or one or more metabolite(s). In an example, the individual was exposed to the one or more drug(s) of abuse (e.g., carfentanil and the like), one or more pesticide(s), one or more chemical warfare agent(s), one or more nerve agent(s), one or more hallucinogen(s), one or more toxin(s), one or more metabolite(s) in a terrorist attack, and combinations thereof.
Statement 28. A method according to any one of Statements 23-27, wherein the individual in need is a human.
Statement 29. A method according to any one of Statements 23-27, where the individual in need is a non-human mammal.
Statement 30. A method according to any one of Statements 27-29, where the drug of abuse is fentanyl.
Statement 31. A method according to Statement 30, wherein the one or more compound(s) are administered at least five minutes after administration of the fentanyl.
Statement 32. A method for prophylaxis and/or therapy of a condition in an individual comprising administering to an individual in need of the prophylaxis and/or the therapy one or more compound(s) according to any of Statements 1-10 and one or more pharmaceutical agent(s), where the compound(s) and the pharmaceutical agent(s) are present as complex (or a composition, which may be a pharmaceutical composition, comprising the complex(es)), where subsequent to the administration the therapy and/or the prophylaxis of the condition in the individual occurs.
Statement 33. A method according to Statement 32, where one or more of the pharmaceutical agent(s) has/have a solubility of less than 100 μM in an aqueous solvent.
Statement 34. A compound according to any one of Statements 1-10, a composition according to any one of Statements 11-15, or a method according to any one of Statements 16-33, where M⁺ is Na⁺, K⁺, H₄N⁺, Et₃NH⁺, Me₄N⁺, (HOCH₂CH₂)₃NH⁺.
Statement 35. A compound according to any one of Statements 1-10, a composition according to any one of Statements 11-15, or a method according to any one of Statements 16-34, where M⁺ is Na⁺.

The following examples are presented to illustrate the present disclosure. They are not intended to be limiting in any manner.

### Example 1

This example provides a description of compounds of the present disclosure, methods of making the compounds, characterization of the compounds, and uses of the compositions.

General experimental details. Starting materials were purchased from commercial suppliers and were used without further purification or were prepared by literature procedures. Melting points were measured on a Meltemp apparatus in open capillary tubes and are uncorrected. IR spectra were recorded on a JASCO FT/IR 4100 spectrometer and are reported in cm⁻¹. ¹H NMR spectra were measured on Bruker instruments operating at 400 or 600 MHz for ¹H and 100 MHz for ¹³C. Mass spectrometry was performed using a JEOL AccuTOF electrospray instrument (ESI). ITC data was collected on a Malvern Microcal PEAQ-ITC instrument.

Synthetic procedures and characterization data.

Host P[5]AS.

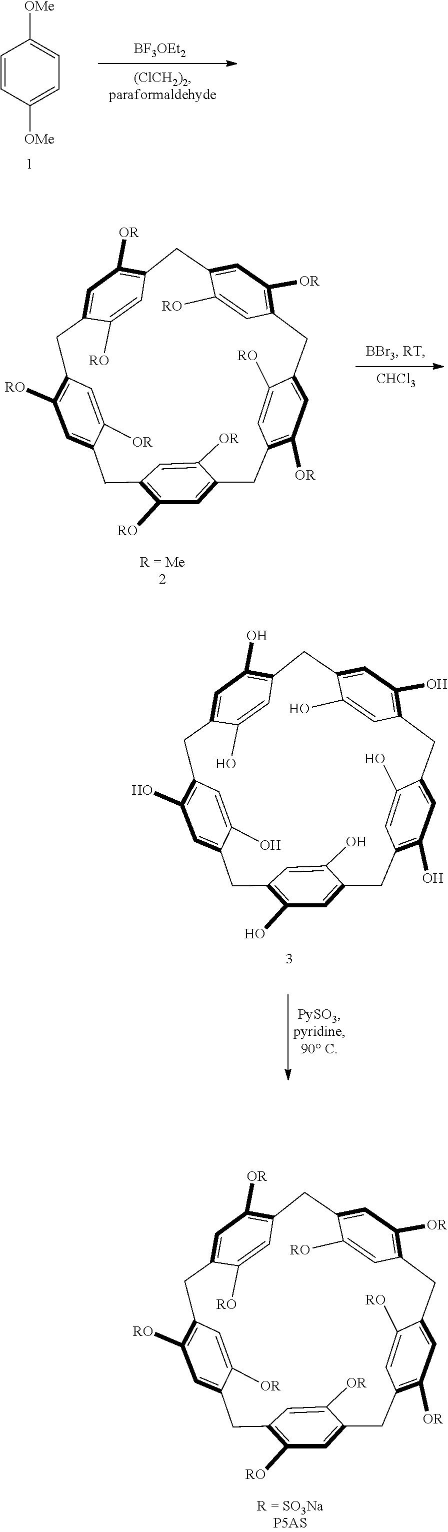

The first two compounds (2 and 3) were synthesized by using methods adapted from methods known in the art. The procedure for the last step was: to a mixture of compound 3 (0.200 g, 0.328 mmol) and pyridine sulfur trioxide complex (1.050 g, 6.56 mmol) was added dry pyridine (10 mL). The resulting mixture was stirred at 90° C. under N2 for 24 hours. The reaction mixture was cooled to RT. The product precipitated out of the solution and was collected by filtration. The solid was slurried in water (5 mL), and the pH was adjusted to 8.4 by slow addition of saturated aqueous NaHCO₃. After addition of EtOH (35 mL), the crude product was collected by centrifugation 7000 rpm×7 min. The precipitate was suspended in ethanol (20 mL×2), sonicated for 30 minutes, and solid collected by centrifugation. The crude solid was redissolved in minimum amount of water (2 ml) and purified by size exclusion chromatography using Sephadex® G25 resin (30 mm×200 mm) and eluted by water. Pure product was collected as the front fractions. After drying under high vacuum, the compound P[5]AS was obtained as a white solid (0.374 g, 0.229 mmol, 70% yield). M.p.>310° C. (decomposed). IR (ATR, cm⁻¹): 3490w, 1630m, 1497m, 1399m, 1234s, 1116s, 1042s, 995m, 941m, 858m, 806m. ¹H NMR (600 MHz, D₂O): 7.31 (s, 10H), 4.00 (s, 10H). ¹³C NMR (150 MHz, D₂O, EtOH as internal reference): 147.4, 134.1, 125.6, 30.8. MS (ESI): m/z 791.78179 ([M-2Na]²⁻), calculated 791.79597.

Host P[5]ACS.

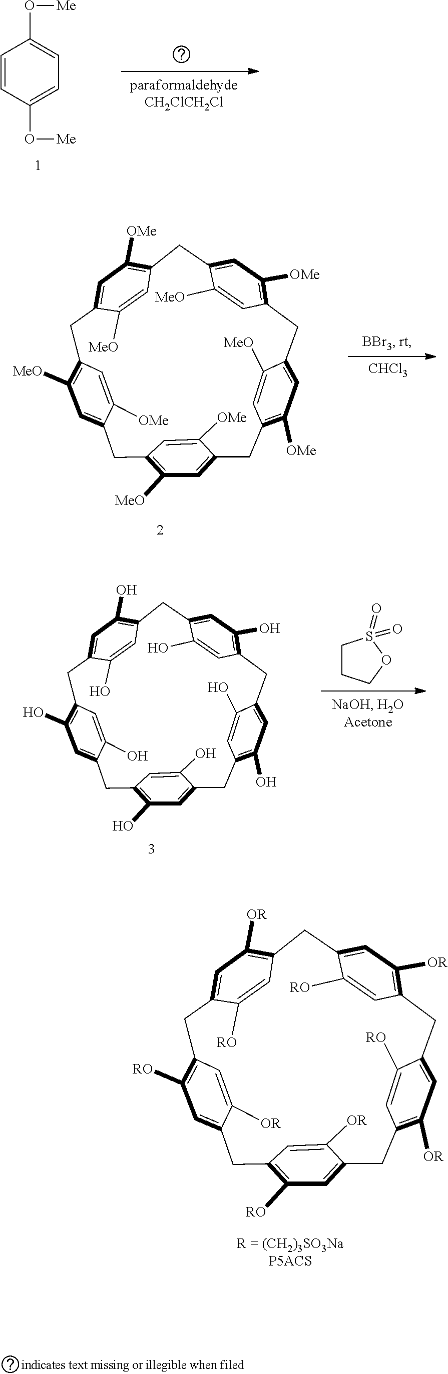

A solution of P[5]A (0.200 g, 0.28 mmol) in NaOH (10 wt %, 2 mL) was treated dropwise with a solution of propane sultone (0.687 g, 5.63 mmol) in acetone (4 mL). This solution was stirred at RT for 5 days (d) and then EtOH (25 mL) was added to the mixture to yield the crude product as a precipitate. The precipitate was obtained by filtration, the solid was dissolved in H₂O (0.5 mL), and then re-precipitated by the addition of EtOH (5 mL) to yield P[5]ACS as a light yellow solid (45 mg, 0.022 mmol, 8%). M.p.>300° C. (decomposed). IR (ATR, cm⁻¹): 3452w, 2936w, 1725m, 1625m, 1479m, 1471m, 1406m, 1181s, 1035s, 951w, 798m, 756m. ¹H NMR (400 MHz, D₂O): 6.76 (s, 10H), 3.90 (m, 10H), 3.86 (s, 10H), 3.68 (m, 10H), 3.05 (m, 20H), 2.08 (m, 20H). ¹³C NMR (150 MHz, D₂O, EtOH as internal reference): δ 150.9, 129.8, 116.7, 68.5, 49.1, 31.1, 25.5. HR-MS (ESI): m/z 1002.03215 ([M-2Na]²⁻), calculated 1002.03072.

Host P[6]AS.

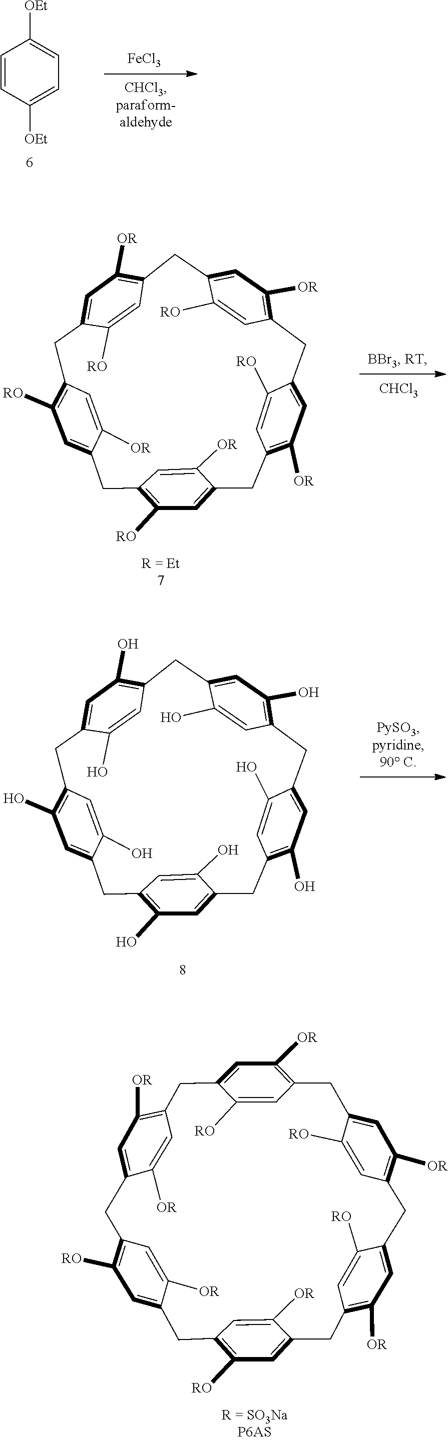

The first two compounds (7 and 8) were synthesized by using methods adapted those known in the art. The procedure for the last step was: to a mixture of compound 8 (0.200 g, 0.27 mmol) and pyridine sulfur trioxide complex (1.090 g, 6.83 mmol) was added dry pyridine (10 mL). The resulting mixture was stirred at 70° C. under N2 for 24 hours. The reaction mixture was cooled to RT. The product precipitated out of the solution and was collected by filtration. The solid was slurried in water (5 mL), and the pH was adjusted to 8.4 by slow addition of saturated aqueous NaHCO₃. After addition of EtOH (35 mL), the crude product was collected by centrifugation 7000 rpm×7 min. The precipitate was suspended in ethanol (20 mL×2), sonicated for 30 minutes, and solid collected by centrifugation. The crude solid was redissolved in minimum amount of water (2 ml) and purified by size exclusion chromatography using Sephadex® G25 resin (30 mm×200 mm) and eluted by water. Pure product was collected as the front fractions. After drying under high vacuum, the compound P[6]AS was obtained as a white solid (0.352 g, 0.18 mmol, 66% yield). M.p.>290° C. (decomposed). IR (ATR, cm⁻¹): 3509w, 1712m, 1630m, 1498m, 1364m, 1237m, 1113s, 1045s, 995m, 942m, 861m, 813m. ¹H NMR (600 MHz, D₂O): 7.35 (s, 12H), 4.11 (s, 12H). ¹³C NMR (150 MHz, D₂O and CD₃OD 10:1): δ 148.1, 133.6, 125.6, 31.5. MS (ESI): m/z 954.7593 ([M-2Na]²⁻), calculated 954.7537.

Host P[6]A8S.

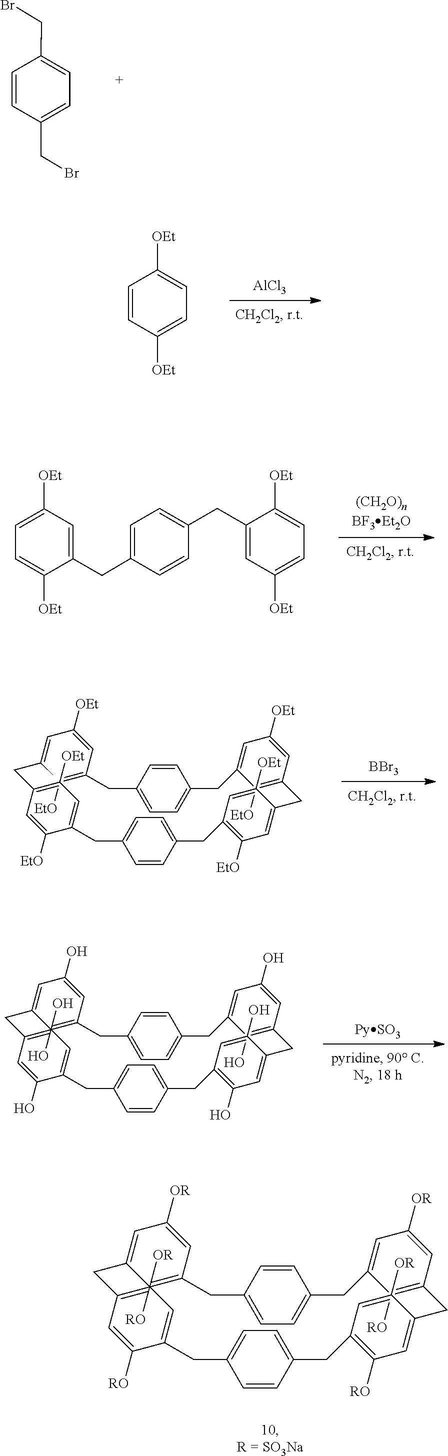

The first three compounds were synthesized by using methods adapted from those known in the art. The procedure for the last step was: to a mixture of compound octahydroxy pillar[6]arene (0.100 g, 0.15 mmol) and pyridine sulfur trioxide complex (0.479 g, 3 mmol) was added dry pyridine (5 mL). The resulting mixture was stirred at 70° C. under N2 for 24 hours. The reaction mixture was cooled to RT. The product precipitated out of the solution and was collected by filtration. The solid was slurried in water (4 mL), and the pH was adjusted to 8.4 by slow addition of saturated aqueous Na₂CO₃. After addition of EtOH (10 mL), the crude product was collected by centrifugation 7000 rpm×7 min. The precipitate was suspended in ethanol (10 mL×2), sonicated for 30 minutes, and solid collected by centrifugation. The crude solid was redissolved in minimum amount of water (0.5 ml) and purified by size exclusion chromatography using Sephadex® G25 resin (30 mm×200 mm) and eluted by water. Pure product was collected as the front fractions. After drying under high vacuum, the compound P[6]A8S (10) was obtained as a white solid (0.075 g, 0.051 mmol, 33% yield). M.p.>285° C. (decomposed). IR (ATR, cm⁻¹): 3491w, 1630m, 1440s, 1234s, 1078m, 1043s, 941m, 878m, 800m, 667m. ¹H NMR (600 MHz, D₂O): 7.39 (s, 4H), 7.25 (s, 4H), 7.04 (s, 8H), 4.07 (s, 4H), 3.99 (s, 8H). ¹³C NMR (150 MHz, D₂O, EtOH as internal reference): δ 147.7, 147.6, 138.7, 134.2, 133.2, 129.2, 125.3, 125.1, 35.7, 31.1. MS (ESI): m/z 718.88334 ([M-2Na]²⁻), calculated 718.88632.

Host P[7]AS.

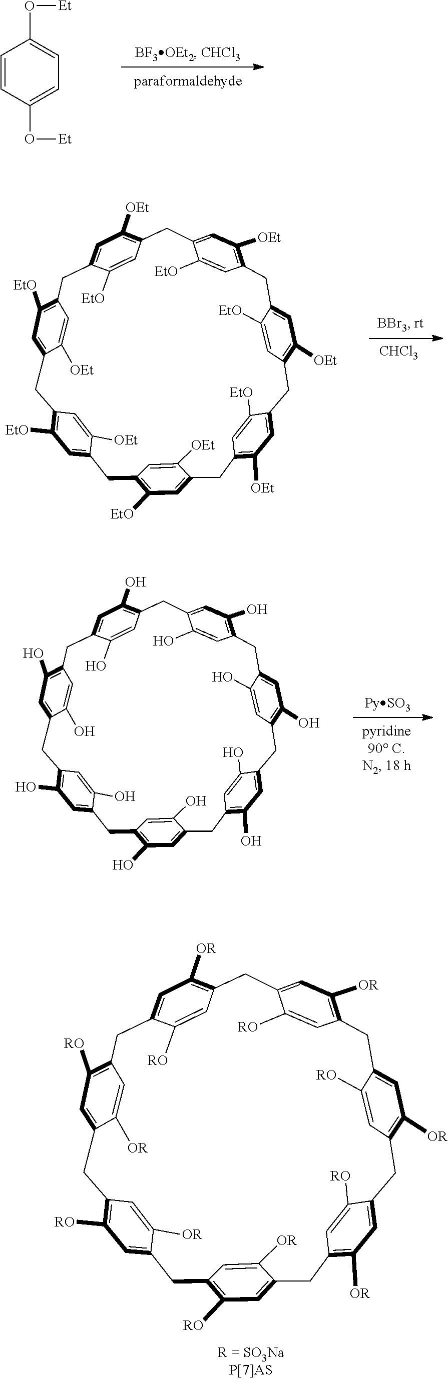

The first two compounds were synthesized by using methods adapted from those known in the art. The procedure for the last step was: to a mixture of compound (HO)₁₄ pillar[7]arene (0.020 g, 0.023 mmol) and pyridine sulfur trioxide complex (0.375 g, 2.34 mmol) was added dry pyridine (3 mL). The resulting mixture was stirred at 70° C. under N2 for 24 hours. The reaction mixture was cooled to RT. The product precipitated out of the solution and was collected by filtration. The solid was slurried in water (1 mL), and the pH was adjusted to 8.4 by slow addition of saturated aqueous Na₂CO₃. After addition of EtOH (10 mL), the crude product was collected by centrifugation 7000 rpm×7 min. The precipitate was suspended in ethanol (10 mL×2), sonicated for 30 minutes, and solid collected by centrifugation. The crude solid was redissolved in minimum amount of water (0.5 ml) and purified by size exclusion chromatography using Sephadex® G25 resin (30 mm×200 mm) and eluted by water. Pure product was collected as the front fractions. After drying under high vacuum, the compound P[7]AS was obtained as a white solid (0.025 g, 0.011 mmol, 46% yield). M.p.>290° C. (decomposed). IR (ATR, cm⁻¹): 3494w, 1624m, 1444s, 1244m, 1102s, 1049s, 995m, 875m, 807m, 614s. ¹H NMR (600 MHz, D₂O): 7.29 (s, 14H), 4.14 (s, 14H). ¹³C NMR (150 MHz, D₂O, Dioxane as external reference): δ 147.6, 132.8, 124.7, 30.8. MS (ESI): m/z 547.36023 ([M-4Na]⁴⁻), calculated 547.36080.

Rim-P[5]AS.

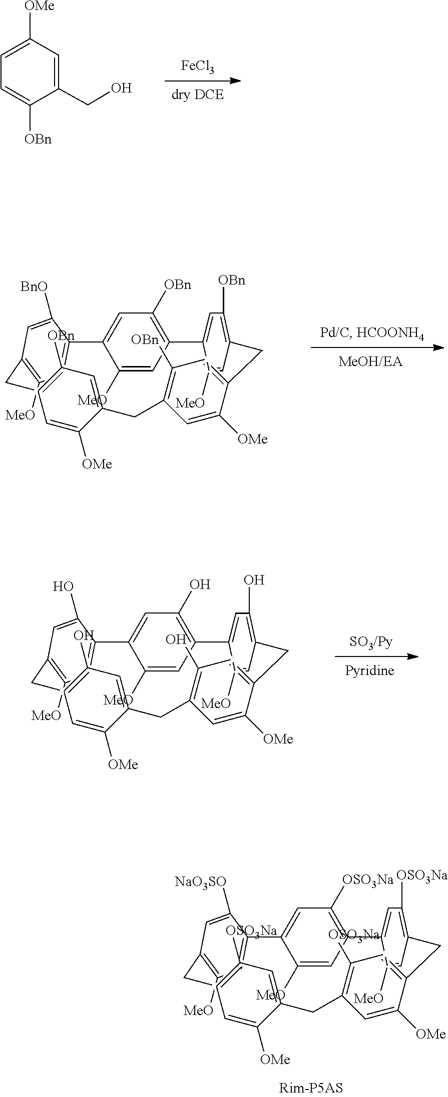

The starting material 2-(Benzyloxy)-5-methoxybenzyl alcohol was synthesized based on methods known in the art. The penta-hydroxy pillar[5]arene compound was synthesized by using the methods known in the art. To a mixture of penta-hydroxy pillar[5]arene (0.200 g, 0.328 mmol) and pyridine sulfur trioxide complex (1.050 g, 6.56 mmol) was added dry pyridine (10 mL). The resulting mixture was stirred at 70° C. under N2 for 24 hours. The reaction mixture was cooled to RT. The product precipitated out of the solution and was collected by filtration. The solid was slurried in water (5 mL), and the pH was adjusted to 9 by slow addition of saturated aqueous NaHCO₃. Addition of EtOH (EtOH/H₂O v/v=2:1) gave a precipitate which was removed by centrifugation (7000 rpm×10 min). The filtrate was collected as the crude product and was redissolved in a minimum amount of water (2 mL) and purified by size exclusion chromatography using Sephadex® G25 resin (5 cm×50 cm) with water as eluent. The front fractions eluting from the column contain pure product. After drying under high vacuum, Rim-P[5]AS was obtained as a white solid (0.374 g, 0.229 mmol, 67% yield, content: ˜92%, determined by using sodium 2-bromoethanesulfonate as ¹H NMR internal standard). M.p.>300° C. (decomposed). ¹H NMR (400 MHz, D₂O): 7.21 (s, 5H), 6.56 (s, 5H), 3.90 (s, 10H), 3.24 (s, 15H). ¹³C NMR (150 MHz, D₂O, EtOH as internal reference): 155.3, 143.2, 134.5, 129.6, 124.7, 114.9, 56.7, 30.6.

Solubility Determination.

Determination of the solubility of P[5]AS in water. Compound P[5]AS was added in excess to 0.5 mL deuterium oxide. This suspension was magnetically stirred at room temperature overnight and then centrifuged (4500 rpm) twice for 10 min each time. Supernatant (50 μL) and sodium 3-(trimethylsilyl)propionate-2,2,3,3-d₄ (TMSP) (10 mM, 50 L in D₂O) were added into 0.4 mL deuterium solvent. The concentration of P[5]AS was measured with ¹H NMR and calculated using sodium 3-(trimethylsilyl)propionate-2,2,3,3-d₄ (TMSP) as internal reference.

Determination of the solubility of P[6]AS in water. Compound P[6]AS was added in excess to 0.5 mL deuterium oxide. This suspension was magnetically stirred at room temperature overnight and then centrifuged (4500 rpm) twice for 10 min each time. Supernatant (50 μL) and sodium 3-(trimethylsilyl)propionate-2,2,3,3-d₄ (TMSP) (10 mM, 50 L in D₂O) were added into 0.4 mL deuterium solvent. The concentration of P[6]AS was measured with ¹H NMR and calculated using sodium 3-(trimethylsilyl)propionate-2,2,3,3-d₄ (TMSP) as internal reference.

Determination of Kₐ between various hosts and cationic guests or drugs of abuse or neuromuscular blocking agents using Isothermal Titration Calorimetry (ITC). All ITC experiments were conducted in the 200 μL working volume of the sample cell of the PEAQ ITC instrument. We used an injection syringe of 40 μL capacity. In each case, the host and guest solutions were prepared in a 20 mM NaH₂PO₄ buffer (pH 7.4). The sample cell was filled to capacity (200 μL) with the host solution and the guest solution was titrated in (first injection=0.4 μL, subsequent 18 injections=2 μL). The binding data was fitted using the 1:1 binding model in MicroCal PEAQ-ITC analysis software. In cases where Kₐ was too large to determine by direct titration, competition ITC titrations were performed where a competitive guest of known Kₐ and ΔH was included in the ITC cell along with the host which was then titrated with the guest whose Kₐ was to be determined.

WP5, WP6, and the following were compounds used in comparative examples:

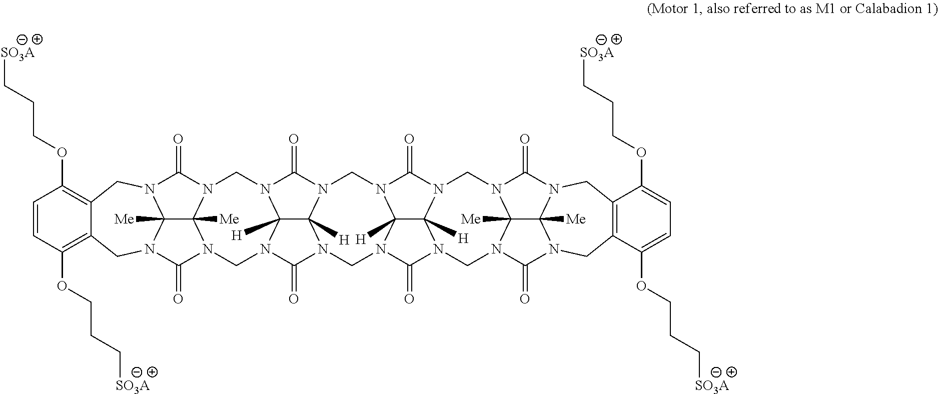

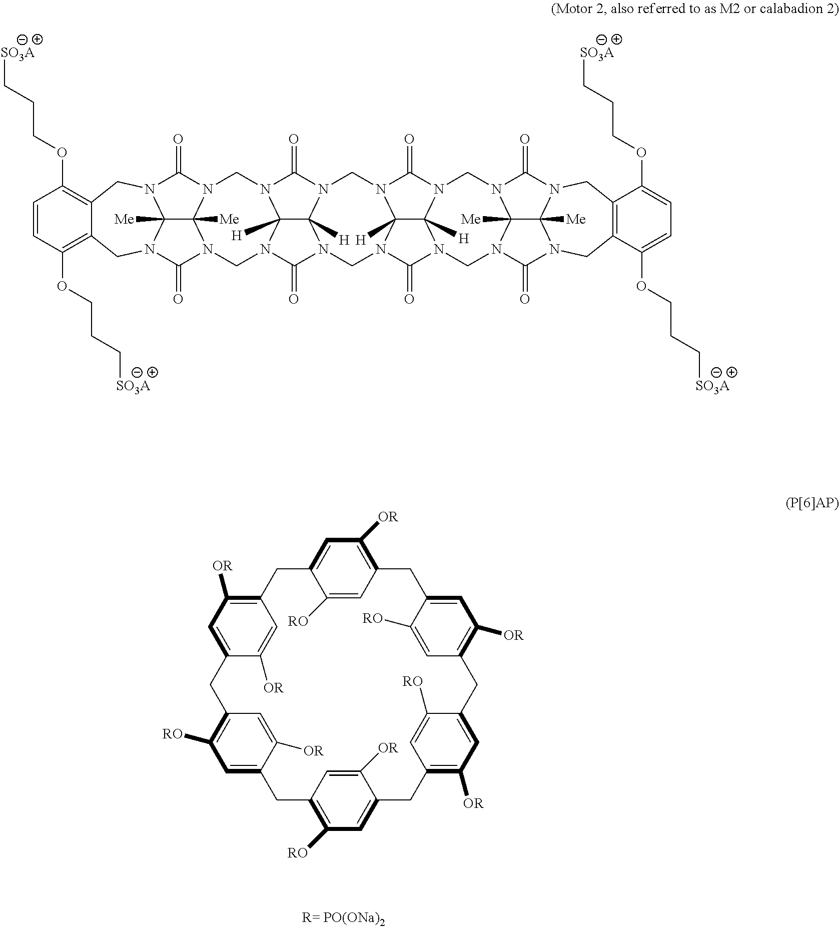

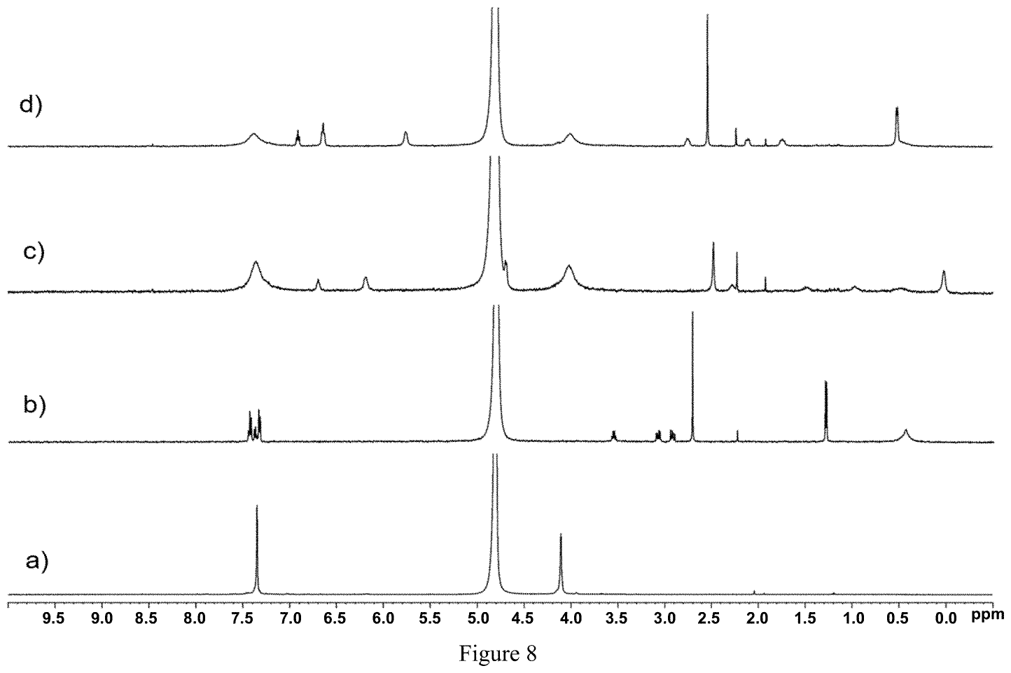

¹H NMR spectra of selected drugs with hosts. FIG. 8 shows an example of a ¹H NMR spectrum of a drug (methamphetamine) with a host (P[6]AS).

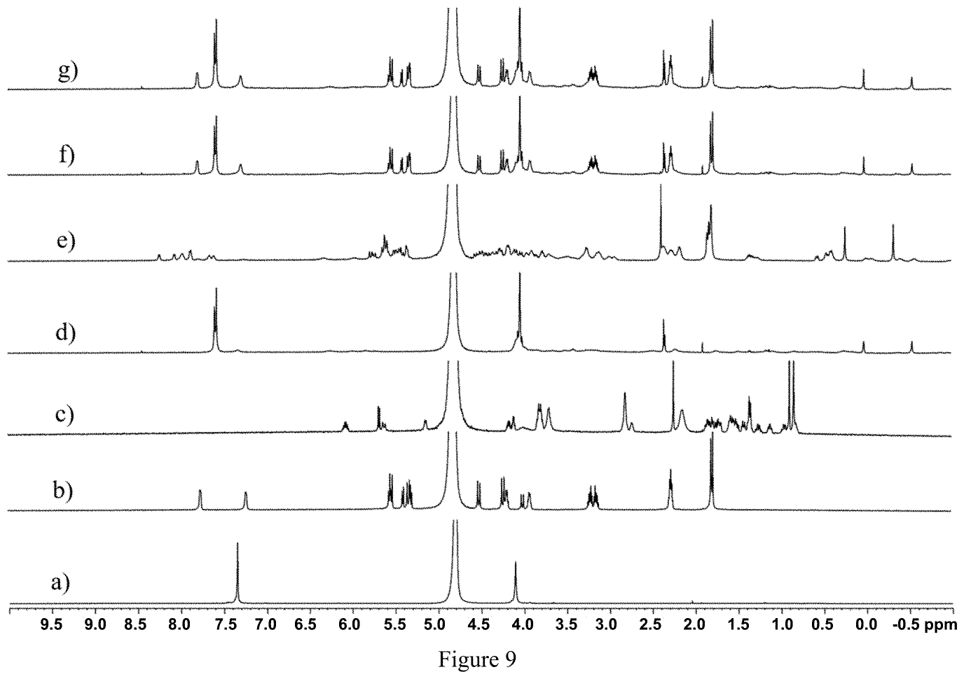

¹H NMR spectra of competition binding. FIG. 9 shows that P[6]AS binds rocuronium stronger a previously known compound (Motor 2, which is also referred to as Calabadion 2).

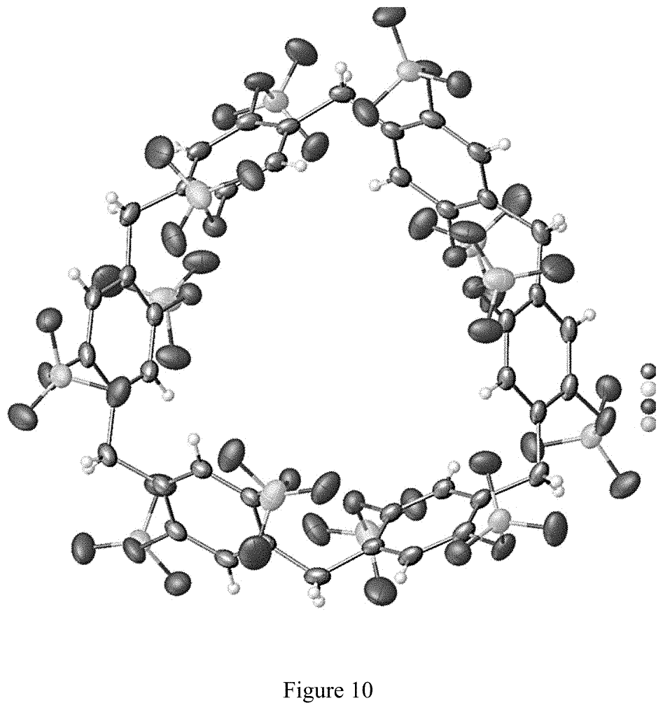

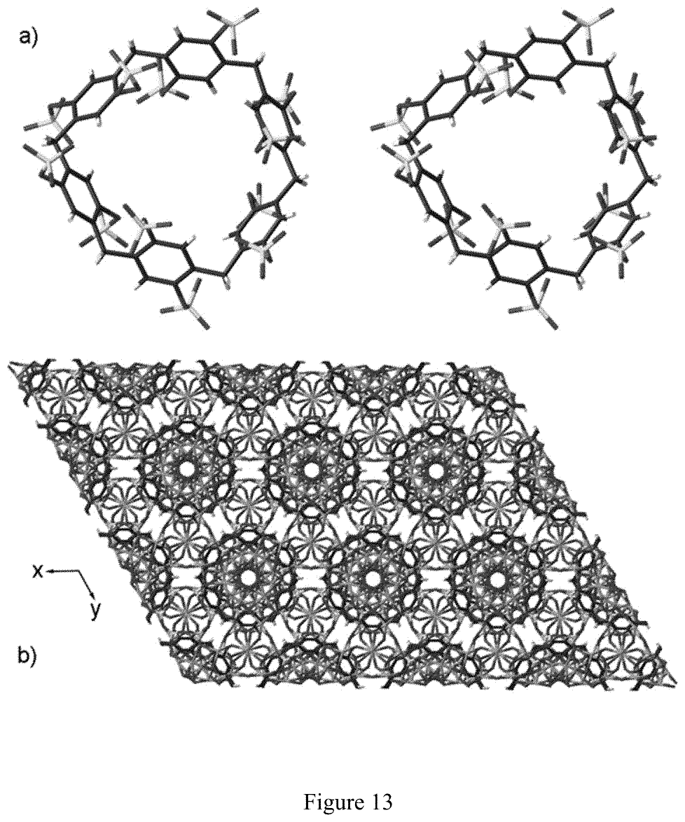

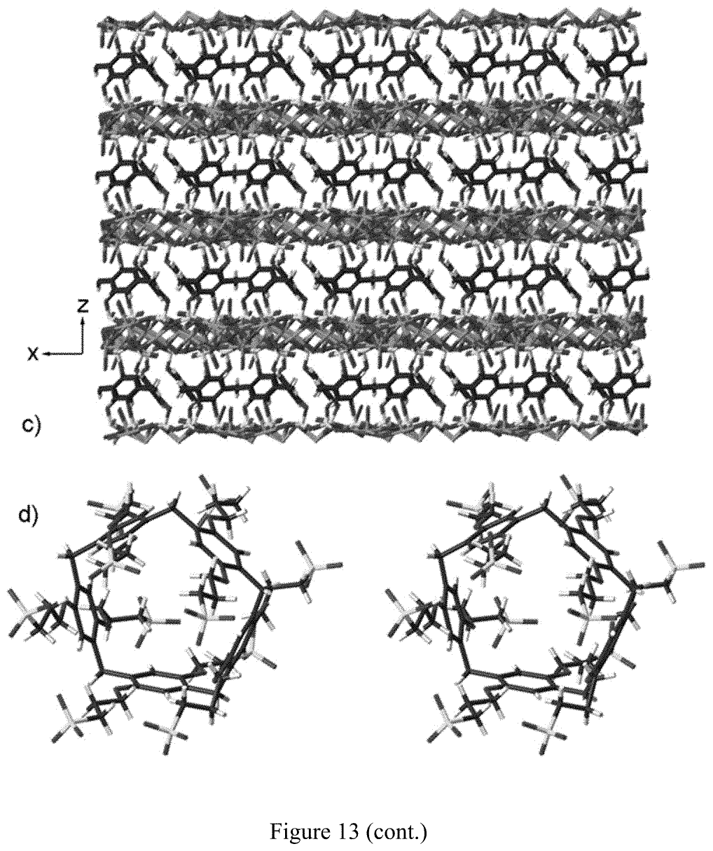

The crystal structures P[6]AS was also determined. FIGS. 10 and 13 show a crystal structure of P[6]AS.

TABLE 1
Crystal data and structure refinement for P[6]AS.
Empirical formula of crystal C_{45.44}H₂₄Na₁₂O_{65.65}S₁₂

| Formula weight | 2280.86 | |
|---|---|---|
| Temperature/K | 150 | (2) |
| Crystal system | trigonal | |
| Space group | P-3c1 | |
| a/Å | 15.112 | (2) |
| b/Å | 15.112 | (2) |
| c/Å | 20.365 | (3) |
| α/° | 90 | |
| β/° | 90 | |
| γ/° | 120 | |
| Volume/Å3 | 4027.8 | (13) |

| | |
|---|---|
| Z | 2 |
| ρcalc g/cm³ | 1.881 |
| μ/mm⁻¹ | 0.519 |
| F(000) | 2292.0 |
| Crystal size/mm³ | 0.28 × 0.14 × 0.08 |
| Radiation | MoKα (λ = 0.71073) |
| 2Θ range for data collection/° | 4 to 53.178 |
| Index ranges | −18 ≤ h ≤ 19, −19 ≤ k ≤ 16, |
| | −25 ≤ 1 ≤ 25 |
| Reflections collected | 23483 |
| Independent reflections | 2810 [Rᵢₙₜ = 0.0602, |
| | R_{sigma} = 0.0410] |
| Data/restraints/parameters | 2810/387/336 |
| Goodness-of-fit on F² | 1.000 |
| Final R indexes [I >= 2σ (I)] | R₁ = 0.0448, wR₂ = 0.0988 |
| Final R indexes [all data] | R₁ = 0.0646, wR₂ = 0.1097 |
| Largest diff. peak/hole/e Å⁻³ 0.53/−0.53 | |

TABLE 2
Fractional Atomic Coordinates and Equivalent Isotropic
Displacement Parameters (Å2) for P[6]AS. Ueq is
defined as 1/3 of the trace of the orthogonalised UIJ tensor.

| Atom | X | | y | | z | | U(eq) | |
|---|---|---|---|---|---|---|---|---|
| Na1 | 0.666667 | | 0.333333 | | 0.50418 | (10) | 0.0407 | (5) |
| Na2 | 0.89555 | (10) | 0.32173 | (10) | 0.50394 | (7) | 0.0465 | (3) |
| Na3 | 0.95059 | (17) | 0.78890 | (17) | 0.44988 | (12) | 0.0502 | (5) |
| O1 | 0.77103 | (19) | 0.4997 | (2) | 0.45146 | (12) | 0.0432 | (7) |
| O2 | 0.9062 | (3) | 0.4595 | (2) | 0.44087 | (14) | 0.0568 | (8) |
| O3 | 0.9417 | (2) | 0.6314 | (2) | 0.42251 | (15) | 0.0515 | (8) |
| S1 | 0.86584 | (19) | 0.52390 | (18) | 0.42005 | (7) | 0.0391 | (2) |
| O4 | 0.83954 | (17) | 0.49388 | (15) | 0.34368 | (11) | 0.0415 | (5) |
| O1A | 0.9727 | (11) | 0.6049 | (14) | 0.4127 | (10) | 0.046 | (3) |
| O2A | 0.7988 | (14) | 0.5652 | (16) | 0.4356 | (9) | 0.043 | (3) |
| O3A | 0.8442 | (18) | 0.4318 | (13) | 0.4474 | (8) | 0.046 | (3) |
| S1A | 0.8672 | (13) | 0.5282 | (13) | 0.4191 | (5) | 0.0391 | (2) |
| O4A | 0.83954 | (17) | 0.49388 | (15) | 0.34368 | (11) | 0.0415 | (5) |
| C1 | 0.8281 | (2) | 0.5602 | (2) | 0.30005 | (15) | 0.0357 | (7) |
| C2 | 0.9039 | (2) | 0.6110 | (2) | 0.25344 | (15) | 0.0326 | (6) |
| C3 | 1 | | 0.6035 | (3) | 0.25 | | 0.0364 | (10) |
| C4 | 0.8884 | (2) | 0.6688 | (2) | 0.20704 | (15) | 0.0329 | (6) |
| C5 | 0.7995 | (2) | 0.6737 | (2) | 0.20759 | (14) | 0.0322 | (6) |
| C6 | 0.7230 | (2) | 0.6236 | (2) | 0.25389 | (15) | 0.0337 | (7) |
| C7 | 0.6223 | (3) | 0.6223 | (3) | 0.25 | | 0.0396 | (10) |
| C8 | 0.7392 | (2) | 0.5664 | (2) | 0.30113 | (16) | 0.0369 | (7) |
| O5 | 0.78551 | (15) | 0.73281 | (15) | 0.15926 | (9) | 0.0340 | (5) |
| S2 | 0.7588 | (5) | 0.6826 | (6) | 0.0870 | (2) | 0.0496 | (5) |
| O6 | 0.6820 | (8) | 0.5770 | (7) | 0.0974 | (6) | 0.074 | (2) |
| O7 | 0.8545 | (7) | 0.7000 | (10) | 0.0598 | (5) | 0.0548 | (19) |
| O8 | 0.7201 | (10) | 0.7419 | (12) | 0.0548 | (4) | 0.0543 | (18) |
| O5A | 0.78551 | (15) | 0.73281 | (15) | 0.15926 | (9) | 0.0340 | (5) |
| S2A | 0.7606 | (7) | 0.6927 | (7) | 0.0846 | (3) | 0.0496 | (5) |
| O6A | 0.6654 | (8) | 0.5974 | (9) | 0.0871 | (6) | 0.057 | (2) |
| O7A | 0.8428 | (11) | 0.6777 | (14) | 0.0624 | (8) | 0.058 | (2) |
| O8A | 0.7537 | (15) | 0.7752 | (11) | 0.0543 | (5) | 0.051 | (2) |
| O1B | 0.8110 | (4) | 0.3730 | (3) | 0.5780 | (2) | 0.0576 | (11) |
| C2B | 0.7898 | (15) | 0.3560 | (15) | 0.6421 | (10) | 0.090 | (7) |
| O3B | 0.7065 | (9) | 0.3682 | (11) | 0.6953 | (5) | 0.089 | (5) |
| C4B | 0.838 | (3) | 0.408 | (3) | 0.580 | (2) | 0.059 | (12) |
| O1C | 0.9547 | (3) | 0.8586 | (3) | 0.3435 | (2) | 0.0871 | (19) |
| O2C | 0.9346 | (8) | 0.7926 | (8) | 0.3985 | (7) | 0.086 | (4) |
| O3C | 0.9615 | (11) | 0.8970 | (11) | 0.2808 | (7) | 0.113 | (5) |
| O1D | 0.0298 | (8) | 0.0909 | (7) | 0.4758 | (5) | 0.083 | (4) |

| | | | | | | |
|---|---|---|---|---|---|---|
| C2D | 0 | 0 | 0.4829 | (5) | 0.025 | (4) |

TABLE 3
Anisotropic Displacement Parameters (Å2) for P[6]AS. The Anisotropic
displacement factor exponent takes the form: −2π²[h²a*²U₁₁ + 2hka*b*U₁₂ + . . .].

| Atom | U11 | | U22 | | U33 | | U23 | | U13 | | U12 | |
|---|---|---|---|---|---|---|---|---|---|---|---|---|
| Na1 | 0.0435 | (7) | 0.0435 | (7) | 0.0351 | (11) | 0 | | 0 | | 0.0218 | (4) |
| Na2 | 0.0409 | (7) | 0.0401 | (7) | 0.0596 | (8) | 0.0049 | (6) | −0.0078 | (6) | 0.0211 | (6) |
| Na3 | 0.0538 | (13) | 0.0572 | (13) | 0.0523 | (13) | −0.0104 | (10) | −0.0086 | (10) | 0.0374 | (11) |
| O1 | 0.0385 | (14) | 0.0419 | (16) | 0.0459 | (14) | 0.0007 | (12) | 0.0090 | (11) | 0.0177 | (12) |
| O2 | 0.065 | (2) | 0.0591 | (18) | 0.0637 | (17) | 0.0257 | (14) | 0.0195 | (15) | 0.0440 | (17) |
| O3 | 0.0451 | (16) | 0.0385 | (15) | 0.0556 | (17) | 0.0123 | (12) | 0.0006 | (13) | 0.0094 | (12) |
| S1 | 0.0396 | (4) | 0.0315 | (4) | 0.0474 | (4) | 0.0098 | (3) | 0.0102 | (3) | 0.0188 | (4) |
| O4 | 0.0561 | (13) | 0.0269 | (10) | 0.0459 | (11) | 0.0104 | (9) | 0.0147 | (10) | 0.0241 | (10) |
| O1A | 0.041 | (4) | 0.042 | (5) | 0.053 | (5) | 0.016 | (5) | 0.008 | (4) | 0.021 | (4) |
| O2A | 0.041 | (4) | 0.042 | (5) | 0.048 | (5) | 0.003 | (4) | 0.007 | (4) | 0.021 | (4) |
| O3A | 0.049 | (5) | 0.037 | (4) | 0.050 | (5) | 0.019 | (4) | 0.011 | (5) | 0.019 | (4) |
| S1A | 0.0396 | (4) | 0.0315 | (4) | 0.0474 | (4) | 0.0098 | (3) | 0.0102 | (3) | 0.0188 | (4) |
| O4A | 0.0561 | (13) | 0.0269 | (10) | 0.0459 | (11) | 0.0104 | (9) | 0.0147 | (10) | 0.0241 | (10) |
| C1 | 0.0430 | (18) | 0.0197 | (14) | 0.0427 | (17) | 0.0044 | (12) | 0.0098 | (14) | 0.0144 | (13) |
| C2 | 0.0351 | (16) | 0.0177 | (13) | 0.0433 | (17) | 0.0006 | (11) | 0.0061 | (13) | 0.0118 | (12) |
| C3 | 0.044 | (3) | 0.0253 | (15) | 0.047 | (3) | 0.0058 | (10) | 0.012 | (2) | 0.0218 | (13) |
| C4 | 0.0309 | (16) | 0.0218 | (14) | 0.0425 | (16) | 0.0040 | (12) | 0.0092 | (13) | 0.0105 | (12) |
| C5 | 0.0335 | (16) | 0.0188 | (13) | 0.0410 | (16) | −0.0005 | (12) | 0.0035 | (12) | 0.0106 | (12) |
| C6 | 0.0275 | (15) | 0.0183 | (13) | 0.0480 | (17) | −0.0027 | (12) | 0.0048 | (13) | 0.0061 | (12) |
| C7 | 0.0267 | (15) | 0.0267 | (15) | 0.052 | (3) | −0.0043 | (11) | 0.0043 | (11) | 0.0033 | (18) |
| C8 | 0.0362 | (17) | 0.0192 | (14) | 0.0477 | (18) | 0.0038 | (13) | 0.0140 | (14) | 0.0082 | (13) |
| O5 | 0.0356 | (11) | 0.0339 | (11) | 0.0360 | (11) | −0.0007 | (8) | 0.0010 | (8) | 0.0199 | (9) |
| S2 | 0.0455 | (5) | 0.0650 | (12) | 0.0464 | (6) | −0.0178 | (6) | −0.0104 | (4) | 0.0336 | (7) |
| O6 | 0.057 | (4) | 0.069 | (4) | 0.093 | (5) | −0.031 | (3) | −0.040 | (3) | 0.030 | (3) |
| O7 | 0.051 | (3) | 0.076 | (4) | 0.049 | (3) | −0.018 | (3) | −0.006 | (3) | 0.041 | (3) |
| O8 | 0.042 | (4) | 0.085 | (5) | 0.045 | (2) | −0.005 | (3) | −0.008 | (2) | 0.038 | (3) |
| O5A | 0.0356 | (11) | 0.0339 | (11) | 0.0360 | (11) | −0.0007 | (8) | 0.0010 | (8) | 0.0199 | (9) |
| S2A | 0.0455 | (5) | 0.0650 | (12) | 0.0464 | (6) | −0.0178 | (6) | −0.0104 | (4) | 0.0336 | (7) |
| O6A | 0.048 | (4) | 0.062 | (4) | 0.066 | (4) | −0.022 | (3) | −0.025 | (3) | 0.032 | (3) |
| O7A | 0.061 | (4) | 0.074 | (5) | 0.053 | (4) | −0.030 | (4) | −0.006 | (3) | 0.045 | (3) |
| O8A | 0.057 | (5) | 0.060 | (4) | 0.042 | (3) | −0.005 | (3) | −0.011 | (4) | 0.033 | (4) |
| O1B | 0.057 | (3) | 0.050 | (3) | 0.0423 | (19) | 0.0027 | (19) | −0.0070 | (18) | 0.009 | (2) |
| C2B | 0.091 | (12) | 0.094 | (13) | 0.091 | (9) | 0.012 | (9) | −0.009 | (9) | 0.050 | (10) |
| O3B | 0.087 | (9) | 0.093 | (10) | 0.094 | (6) | −0.018 | (6) | −0.028 | (5) | 0.048 | (6) |
| C4B | 0.056 | (15) | 0.061 | (15) | 0.050 | (14) | 0.000 | (9) | −0.002 | (9) | 0.022 | (10) |
| O1C | 0.068 | (3) | 0.099 | (3) | 0.115 | (4) | 0.028 | (3) | 0.014 | (2) | 0.057 | (2) |
| O2C | 0.087 | (7) | 0.072 | (6) | 0.114 | (8) | 0.008 | (5) | 0.014 | (6) | 0.052 | (5) |
| O3C | 0.120 | (9) | 0.107 | (8) | 0.121 | (8) | 0.015 | (6) | 0.017 | (6) | 0.064 | (7) |
| O1D | 0.093 | (6) | 0.057 | (6) | 0.107 | (7) | −0.007 | (4) | −0.019 | (5) | 0.043 | (5) |

| | | | | | | | | | | |
|---|---|---|---|---|---|---|---|---|---|---|
| C2D | 0.018 | (4) | 0.018 | (4) | 0.040 | (8) | 0 | 0 | 0.0088 | (19) |

TABLE 4
Bond Lengths for P[6]AS.

| Atom | Atom | Length/Å | | Atom | Atom | Length/Å | |
|---|---|---|---|---|---|---|---|
| Na1 | O11 | 2.449 | (3) | O3 | S1 | 1.447 | (3) |
| Na1 | O1 | 2.449 | (3) | S1 | O4 | 1.613 | (3) |
| Na1 | O12 | 2.449 | (3) | O4 | C1 | 1.413 | (4) |
| Na1 | O1B1 | 2.464 | (5) | C1 | C2 | 1.387 | (4) |
| Na1 | O1B | 2.464 | (5) | C1 | C8 | 1.393 | (4) |
| Na1 | O1B2 | 2.464 | (5) | C2 | C4 | 1.384 | (4) |
| Na2 | O83 | 2.262 | (10) | C2 | C3 | 1.514 | (4) |
| Na2 | O1B | 2.345 | (6) | C4 | C5 | 1.381 | (4) |
| Na2 | O2A1 | 2.347 | (14) | C5 | C6 | 1.387 | (4) |
| Na2 | O2 | 2.382 | (3) | C5 | O5 | 1.414 | (3) |
| Na2 | O11 | 2.435 | (3) | C6 | C8 | 1.394 | (4) |
| Na2 | O74 | 2.548 | (10) | C6 | C7 | 1.514 | (4) |
| Na2 | O35 | 2.655 | (3) | O5 | S2 | 1.611 | (5) |
| Na3 | O1D6 | 2.268 | (9) | S2 | O6 | 1.445 | (6) |
| Na3 | O3 | 2.383 | (4) | S2 | O7 | 1.446 | (5) |
| Na3 | O1C | 2.395 | (5) | S2 | O8 | 1.448 | (5) |
| Na3 | O1D1 | 2.450 | (11) | O1B | C2B | 1.34 | (2) |
| Na3 | O77 | 2.632 | (10) | O1C | O3C | 1.387 | (13) |
| Na3 | O88 | 2.645 | (10) | O1C | O2C | 1.426 | (13) |
| Na3 | O78 | 2.685 | (13) | O3C | O3C7 | 1.61 | (3) |
| Na3 | O1D9 | 2.689 | (11) | O1D | C2D | 1.221 | (9) |
| Na3 | O810 | 2.786 | (15) | O1D | C2D11 | 1.476 | (10) |
| O1 | S1 | 1.439 | (3) | O1D | O1D12 | 1.563 | (14) |
| O2 | S1 | 1.447 | (4) | O1D | O1D13 | 1.563 | (14) |

¹1 + Y − X, 1 − X, +Z;
²1 − Y, +X − Y, +Z;
³1 − Y + X, 1 − Y, 1/2 − Z;
⁴+X, +X − Y, 1/2 + Z;
⁵2 − X, 1 − Y, 1 − Z;
⁶1 − X, 1 − Y, 1 − Z;
⁷2 − X, 1 − X + Y, 1/2 − Z;
⁸1 + Y − X, +Y, 1/2 + Z;
⁹1 − Y, 1 + X − Y, +Z;
¹⁰+Y, +X, 1/2 − Z;
¹¹−X, −Y, 1 − Z;
¹²−Y + X, +X, 1 − Z;
¹³+Y, −X + Y, 1 − Z

TABLE 5
Bond Angles for P[6]AS.

| Atom | Atom | Atom | Angle/° | | Atom | Atom | Atom | Angle/° | |
|---|---|---|---|---|---|---|---|---|---|
| O11 | Na1 | O1 | 102.22 | (9) | O1 | S1 | O2 | 113.6 | (2) |
| O11 | Na1 | O12 | 102.22 | (9) | O3 | S1 | O2 | 112.4 | (3) |
| O1 | Na1 | O12 | 102.22 | (9) | O1 | S1 | O4 | 106.44 | (18) |
| O11 | Na1 | O1B1 | 88.89 | (12) | O3 | S1 | O4 | 107.14 | (19) |
| O1 | Na1 | O1B1 | 167.25 | (14) | O2 | S1 | O4 | 102.75 | (19) |
| O12 | Na1 | O1B1 | 81.19 | (14) | C1 | O4 | S1 | 120.5 | (2) |
| O11 | Na1 | O1B | 81.19 | (13) | C2 | C1 | C8 | 122.0 | (3) |
| O1 | Na1 | O1B | 88.88 | (12) | C2 | C1 | O4 | 117.6 | (3) |
| O12 | Na1 | O1B | 167.25 | (14) | C8 | C1 | O4 | 120.2 | (3) |
| O1B1 | Na1 | O1B | 86.63 | (18) | C4 | C2 | C1 | 117.6 | (3) |
| O11 | Na1 | O1B2 | 167.25 | (14) | C4 | C2 | C3 | 119.2 | (2) |
| O1 | Na1 | O1B2 | 81.19 | (14) | C1 | C2 | C3 | 123.2 | (3) |
| O12 | Na1 | O1B2 | 88.88 | (12) | C2 | C3 | C27 | 112.6 | (3) |
| O1B1 | Na1 | O1B2 | 86.63 | (18) | C5 | C4 | C2 | 120.3 | (3) |
| O1B | Na1 | O1B2 | 86.63 | (18) | C4 | C5 | C6 | 122.9 | (3) |
| O83 | Na2 | O1B | 171.3 | (3) | C4 | C5 | O5 | 119.0 | (3) |
| O83 | Na2 | O2 | 106.0 | (3) | C6 | C5 | O5 | 118.1 | (3) |
| O1B | Na2 | O2 | 82.44 | (14) | C5 | C6 | C8 | 116.8 | (3) |
| O83 | Na2 | O11 | 99.0 | (3) | C5 | C6 | C7 | 122.0 | (3) |
| O1B | Na2 | O11 | 83.93 | (14) | C8 | C6 | C7 | 121.0 | (2) |
| O2 | Na2 | O11 | 82.22 | (11) | C6 | C7 | C610 | 118.8 | (3) |
| O83 | Na2 | O74 | 74.2 | (3) | C1 | C8 | C6 | 120.4 | (3) |
| O1B | Na2 | O74 | 97.9 | (3) | C5 | O5 | S2 | 114.8 | (4) |
| O2 | Na2 | O74 | 169.6 | (3) | O6 | S2 | O7 | 115.8 | (6) |
| O11 | Na2 | O74 | 87.5 | (3) | O6 | S2 | O8 | 113.6 | (5) |
| O3A | Na2 | O74 | 151.8 | (6) | O7 | S2 | O8 | 113.0 | (6) |
| C4B | Na2 | O74 | 105.8 | (11) | O6 | S2 | O5 | 105.1 | (5) |
| O83 | Na2 | O35 | 77.1 | (2) | O7 | S2 | O5 | 105.8 | (5) |
| O1B | Na2 | O35 | 97.67 | (15) | O8 | S2 | O5 | 101.9 | (4) |
| O2 | Na2 | O35 | 114.95 | (12) | S2 | O7 | Na211 | 129.1 | (8) |
| O11 | Na2 | O35 | 162.82 | (10) | S2 | O7 | Na37 | 142.5 | (8) |
| O74 | Na2 | O35 | 75.3 | (3) | Na211 | O7 | Na37 | 88.1 | (2) |
| O2C | Na3 | O1D6 | 123.2 | (6) | S2 | O7 | Na312 | 94.4 | (6) |
| O2C | Na3 | O3 | 85.6 | (6) | Na211 | O7 | Na312 | 96.9 | (3) |
| O1D6 | Na3 | O3 | 151.2 | (3) | Na37 | O7 | Na312 | 83.4 | (3) |
| O1D6 | Na3 | O1C | 106.7 | (3) | S2 | O8 | Na213 | 126.0 | (9) |
| O3 | Na3 | O1C | 101.75 | (15) | S2 | O8 | Na312 | 96.0 | (4) |
| O2C | Na3 | O1D1 | 101.2 | (6) | Na213 | O8 | Na312 | 94.1 | (3) |
| O3 | Na3 | O1D1 | 146.6 | (2) | S2 | O8 | Na310 | 132.7 | (7) |
| O1C | Na3 | O1D1 | 81.4 | (3) | Na213 | O8 | Na310 | 101.3 | (3) |
| O2C | Na3 | O77 | 98.8 | (6) | Na312 | O8 | Na310 | 81.3 | (4) |
| O1D6 | Na3 | O77 | 96.5 | (4) | C2B | O1B | Na2 | 133.0 | (9) |
| O3 | Na3 | O77 | 78.5 | (3) | C2B | O1B | Na1 | 116.8 | (9) |
| O1C | Na3 | O77 | 90.6 | (2) | Na2 | O1B | Na1 | 95.11 | (17) |
| O1D1 | Na3 | O77 | 68.1 | (3) | Na2 | C4B | Na1 | 86.4 | (14) |
| O2C | Na3 | O88 | 158.0 | (6) | O2C | O1C | Na3 | 15.0 | (5) |
| O1D6 | Na3 | O88 | 76.6 | (4) | Na3 | O2C | O1C | 145.1 | (10) |
| O3 | Na3 | O88 | 75.4 | (3) | O1C | O3C | O3C7 | 139.3 | (13) |
| O1C | Na3 | O88 | 157.5 | (2) | C2D | O1D | C2D14 | 28.0 | (8) |
| O1D1 | Na3 | O88 | 89.2 | (4) | C2D | O1D | O1D15 | 62.6 | (4) |
| O2C | Na3 | O78 | 137.9 | (6) | C2D14 | O1D | O1D15 | 47.3 | (3) |
| O1D6 | Na3 | O78 | 69.6 | (4) | C2D | O1D | O1D16 | 62.6 | (4) |
| O3 | Na3 | O78 | 88.4 | (3) | C2D14 | O1D | O1D16 | 47.3 | (3) |
| O1C | Na3 | O78 | 148.5 | (2) | O1D15 | O1D | O1D16 | 84.4 | (8) |
| O1D1 | Na3 | O78 | 106.4 | (3) | C2D | O1D | Na36 | 130.5 | (8) |
| O77 | Na3 | O78 | 120.7 | (2) | C2D14 | O1D | Na36 | 102.8 | (6) |
| O2C | Na3 | O1D9 | 89.7 | (6) | O1D15 | O1D | Na36 | 77.1 | (7) |
| O3 | Na3 | O1D9 | 165.4 | (2) | O1D16 | O1D | Na36 | 87.1 | (7) |
| O1C | Na3 | O1D9 | 76.9 | (2) | C2D | O1D | Na32 | 102.7 | (6) |
| O77 | Na3 | O1D9 | 115.8 | (3) | C2D14 | O1D | Na32 | 106.5 | (5) |
| O88 | Na3 | O1D9 | 111.4 | (4) | O1D15 | O1D | Na32 | 148.7 | (7) |
| O78 | Na3 | O1D9 | 86.0 | (3) | O1D16 | O1D | Na32 | 64.5 | (5) |
| S1 | O1 | Na22 | 143.8 | (2) | Na36 | O1D | Na32 | 97.1 | (4) |
| S1 | O1 | Na1 | 120.26 | (18) | C2D | O1D | Na317 | 90.7 | (5) |
| Na22 | O1 | Na1 | 93.24 | (9) | C2D14 | O1D | Na317 | 95.9 | (5) |
| S1 | O2 | Na2 | 151.5 | (2) | O1D15 | O1D | Na317 | 57.4 | (4) |
| S1 | O3 | Na3 | 138.3 | (2) | O1D16 | O1D | Na317 | 141.1 | (6) |
| S1 | O3 | Na25 | 115.3 | (2) | Na36 | O1D | Na317 | 90.7 | (3) |
| Na3 | O3 | Na25 | 91.12 | (11) | Na32 | O1D | Na317 | 153.9 | (4) |

| | | | | |
|---|---|---|---|---|
| O1 | S1 | O3 | 113.6 | (2) |

¹1 + Y − X, 1 − X, +Z;
²1 − Y, +X − Y, +Z;
³1 − Y + X, 1 − Y, 1/2 − Z;
⁴+X, +X − Y, 1/2 + Z;
⁵2 − X, 1 − Y, 1 − Z;
⁶1 − X, 1 − Y, 1 − Z;
⁷2 − X, 1 − X + Y, 1/2 − Z;
⁸1 + Y − X, +Y, 1/2 + Z;
⁹1 − Y, 1 + X − Y, +Z;
¹⁰+Y, +X, 1/2 − Z;
¹¹+X, +X − Y, −1/2 + Z;
¹²1 + Y − X, +Y, −1/2 + Z;
¹³−Y + X, 1 − Y, 1/2 − Z;
¹⁴−X, −Y, 1 − Z;
¹⁵−Y + X , +X, 1 − Z;
¹⁶+Y, −X + Y, 1 − Z;
¹⁷+Y − X, 1 − X, +Z

TABLE 6
Torsion Angles for P[6]AS.

| A | B | C | D | Angle/° | | A | B | C | D | Angle/° | |
|---|---|---|---|---|---|---|---|---|---|---|---|
| Na21 | O1 | S1 | O3 | 6.3 | (4) | O5 | S2 | O7 | Na36 | −126.4 | (4) |
| Na1 | O1 | S1 | O3 | −148.93 | (19) | O6 | S2 | O8 | Na27 | −18.9 | (8) |
| Na21 | O1 | S1 | O2 | 136.3 | (3) | O7 | S2 | O8 | Na27 | 115.5 | (7) |
| Na1 | O1 | S1 | O2 | −18.9 | (3) | O5 | S2 | O8 | Na27 | −131.4 | (6) |
| Na21 | O1 | S1 | O4 | −111.4 | (3) | O6 | S2 | O8 | Na36 | −118.5 | (6) |
| Na1 | O1 | S1 | O4 | 93.4 | (2) | O7 | S2 | O8 | Na36 | 16.0 | (6) |
| Na3 | O3 | S1 | O1 | −11.8 | (4) | O5 | S2 | O8 | Na36 | 129.0 | (5) |
| Na22 | O3 | S1 | O1 | 112.5 | (2) | O6 | S2 | O8 | Na34 | 157.9 | (7) |
| Na3 | O3 | S1 | O2 | −142.4 | (3) | O7 | S2 | O8 | Na34 | −67.7 | (9) |
| Na22 | O3 | S1 | O2 | −18.1 | (3) | O5 | S2 | O8 | Na34 | 45.4 | (7) |
| Na3 | O3 | S1 | O4 | 105.5 | (3) | O1D8 | Na3 | O2C | O1C | 37.0 | (19) |
| Na22 | O3 | S1 | O4 | −130.24 | (17) | O3 | Na3 | O2C | O1C | −144.4 | (15) |
| Na2 | O2 | S1 | O1 | 1.7 | (5) | O1D9 | Na3 | O2C | O1C | 2.5 | (16) |
| Na2 | O2 | S1 | O3 | 132.3 | (4) | O73 | Na3 | O2C | O1C | −66.7 | (16) |
| Na2 | O2 | S1 | O4 | −112.8 | (4) | O810 | Na3 | O2C | O1C | −114.3 | (18) |
| O1 | S1 | O4 | C1 | 80.0 | (3) | O710 | Na3 | O2C | O1C | 132.9 | (13) |
| O3 | S1 | O4 | C1 | −41.8 | (3) | O1D11 | Na3 | O2C | O1C | 49.4 | (15) |
| O2 | S1 | O4 | C1 | −160.4 | (3) | O84 | Na3 | O2C | O1C | 116.6 | (16) |
| S1 | O4 | C1 | C2 | 109.1 | (3) | C2D12 | Na3 | O2C | O1C | 25.9 | (16) |
| S1 | O4 | C1 | C8 | −76.1 | (4) | O1D13 | O1D | C2D | C2D14 | −49.2 | (5) |
| C8 | C1 | C2 | C4 | −0.3 | (4) | O1D15 | O1D | C2D | C2D14 | 49.2 | (5) |
| O4 | C1 | C2 | C4 | 174.4 | (3) | Na38 | O1D | C2D | C2D14 | −9.8 | (8) |
| C8 | C1 | C2 | C3 | −178.4 | (3) | Na31 | O1D | C2D | C2D14 | 101.3 | (3) |
| O4 | C1 | C2 | C3 | −3.7 | (4) | Na316 | O1D | C2D | C2D14 | −101.3 | (2) |
| C4 | C2 | C3 | C23 | 52.2 | (2) | C2D14 | O1D | C2D | O1D17 | −101.6 | (11) |
| C1 | C2 | C3 | C23 | −129.7 | (3) | O1D13 | O1D | C2D | O1D17 | −150.8 | (10) |
| C1 | C2 | C4 | C5 | −0.7 | (4) | O1D15 | O1D | C2D | O1D17 | −52.4 | (14) |
| C3 | C2 | C4 | C5 | 177.5 | (3) | Na38 | O1D | C2D | O1D17 | −111.4 | (13) |
| C2 | C4 | C5 | C6 | 0.9 | (5) | Na31 | O1D | C2D | O1D17 | −0.3 | (14) |
| C2 | C4 | C5 | O5 | −179.5 | (2) | Na316 | O1D | C2D | O1D17 | 157.1 | (10) |
| C4 | C5 | C6 | C8 | −0.1 | (4) | C2D14 | O1D | C2D | O1D18 | 101.6 | (11) |
| O5 | C5 | C6 | C8 | −179.6 | (2) | O1D13 | O1D | C2D | O1D18 | 52.4 | (14) |
| C4 | C5 | C6 | C7 | −174.4 | (3) | O1D15 | O1D | C2D | O1D18 | 150.8 | (10) |
| O5 | C5 | C6 | C7 | 6.0 | (4) | Na38 | O1D | C2D | O1D18 | 91.8 | (14) |
| C5 | C6 | C7 | C64 | −54.9 | (2) | Na31 | O1D | C2D | O1D18 | −157.1 | (8) |
| C8 | C6 | C7 | C64 | 131.0 | (3) | Na316 | O1D | C2D | O1D18 | 0.3 | (13) |
| C2 | C1 | C8 | C6 | 1.1 | (5) | C2D14 | O1D | C2D | O1D13 | 49.2 | (5) |
| O4 | C1 | C8 | C6 | −173.4 | (3) | O1D15 | O1D | C2D | O1D13 | 98.3 | (10) |
| C5 | C6 | C8 | C1 | −0.9 | (4) | Na38 | O1D | C2D | O1D13 | 39.4 | (9) |
| C7 | C6 | C8 | C1 | 173.5 | (3) | Na31 | O1D | C2D | O1D13 | 150.4 | (8) |
| C4 | C5 | O5 | S2 | 74.4 | (4) | Na316 | O1D | C2D | O1D13 | −52.1 | (3) |
| C6 | C5 | O5 | S2 | −106.1 | (4) | C2D14 | O1D | C2D | O1D15 | −49.2 | (5) |
| C5 | O5 | S2 | O6 | 45.2 | (7) | O1D13 | O1D | C2D | O1D15 | −98.3 | (10) |
| C5 | O5 | S2 | O7 | −77.8 | (8) | Na38 | O1D | C2D | O1D15 | −58.9 | (10) |
| C5 | O5 | S2 | O8 | 163.9 | (6) | Na31 | O1D | C2D | O1D15 | 52.1 | (3) |
| O6 | S2 | O7 | Na25 | 15.2 | (11) | Na316 | O1D | C2D | O1D15 | −150.5 | (7) |
| O8 | S2 | O7 | Na25 | −118.2 | (8) | C2D14 | O1D | C2D | O1D14 | −0.004 | (1) |
| O5 | S2 | O7 | Na25 | 131.1 | (7) | O1D13 | O1D | C2D | O1D14 | −49.2 | (5) |
| O6 | S2 | O7 | Na33 | −157.3 | (12) | O1D15 | O1D | C2D | O1D14 | 49.2 | (5) |
| O8 | S2 | O7 | Na33 | 69.2 | (14) | Na38 | O1D | C2D | O1D14 | −9.8 | (8) |
| O5 | S2 | O7 | Na33 | −41.4 | (14) | Na31 | O1D | C2D | O1D14 | 101.3 | (3) |
| O6 | S2 | O7 | Na36 | 117.7 | (5) | Na316 | O1D | C2D | O1D14 | −101.3 | (2) |

| | | | | | |
|---|---|---|---|---|---|
| O8 | S2 | O7 | Na36 | −15.7 | (6) |

¹1 − Y, +X − Y, +Z;
²2 − X, 1 − Y, 1 − Z;
³2 − X, 1 − X + Y, 1/2 − Z;
⁴+Y, +X, 1/2 − Z;
⁵+X, +X − Y, −1/2 + Z;
⁶1 + Y − X, +Y, −1/2 + Z;
⁷−Y + X, 1 − Y, 1/2 − Z;
⁸1 − X, 1 − Y, 1 − Z;
⁹1 + Y − X, 1 − X, +Z;
¹⁰1 + Y − X, +Y, 1/2 + Z;
¹¹1 − Y, 1 + X − Y, +Z;
¹²1 + X, 1 + Y, +Z;
¹³−Y + X , +X, 1 − Z;
¹⁴−X, −Y, 1 − Z;
¹⁵+Y, −X + Y, 1 − Z;
¹⁶+Y − X, 1 − X, +Z;
¹⁷+Y − X, −X, +Z;
¹⁸−Y, +X − Y, +Z

TABLE 7
Hydrogen Atom Coordinates and Isotropic Displacement
Parameters (Å2) for P[6]AS.

| Atom | Z | y | z | U(eq) |
|---|---|---|---|---|
| H3 | 1.002(2) | 0.569(2) | 0.2874(14) | 0.039(9) |
| H4 | 0.937(2) | 0.703(2) | 0.1741(14) | 0.026(7) |
| H6 | 0.691(2) | 0.532(2) | 0.3338(14) | 0.032(8) |
| H7 | 0.590(2) | 0.588(2) | 0.2112(14) | 0.035(8) |

TABLE 8
Atomic Occupancy for P[6]AS.

| Atom | Occupancy | | Atom | Occupancy | | Atom | Occupancy | |
|---|---|---|---|---|---|---|---|---|
| Na3 | 0.6667 | | O1 | 0.887 | (5) | O2 | 0.887 | (5) |
| O3 | 0.887 | (5) | S1 | 0.887 | (5) | O4 | 0.887 | (5) |
| O1A | 0.113 | (5) | O2A | 0.113 | (5) | O3A | 0.113 | (5) |
| S1A | 0.113 | (5) | O4A | 0.113 | (5) | O5 | 0.58 | (3) |
| S2 | 0.58 | (3) | O6 | 0.58 | (3) | O7 | 0.58 | (3) |
| O8 | 0.58 | (3) | O5A | 0.42 | (3) | S2A | 0.42 | (3) |
| O6A | 0.42 | (3) | O7A | 0.42 | (3) | O8A | 0.42 | (3) |
| O1B | 0.85 | | C2B | 0.285 | (13) | O3B | 0.308 | (7) |
| C4B | 0.15 | | O1C | 0.808 | (11) | O2C | 0.317 | (10) |
| O3C | 0.306 | (10) | O1D | 0.353 | (9) | C2D | 0.412 | (17) |

Experimental. A suitable single crystal of P[6]AS was selected and measured on a Bruker Smart Apex2 diffractometer. The crystal corresponded to C_{45.44}H₂₄Na₁₂O_{65.65}S₁₂. The crystal was kept at 150(2) K during data collection. The integral intensity were correct for absorption using SADABS software using multi-scan method. Resulting minimum and maximum transmission are 0.634 and 0.959 respectively. The structure was solved with the ShelXT-2014 (Sheldrick, 2015a) program and refined with the ShelXL-2015 (Sheldrick, 2015c) program and least-square minimisation using ShelX software package. Number of restraints used=387.

Crystal structure determination. Crystal data for C_{45.44}H₂₄Na₁₂O_{65.65}S₁₂ (M=2280.86 g/mol): trigonal, space group P-3cl (no. 165), a=15.112(2) Å, c=20.365(3) Å, V=4027.8(13) Å3, Z=2, T=150(2) K, μ(MoKα)=0.519 mm⁻¹, D_{calc}=1.881 g/cm³, 23483 reflections measured (4°≤2Θ≤53.178°), 2810 unique (Rᵢₙₜ=0.0602, R_{sig}=0.0410) which were used in all calculations. The final R₁ was 0.0448 (I>2σ(I)) and wR₂ was 0.1097 (all data).

Refinement details. H atoms (except those in disordered solvent) were located from difference Fourier map and freely refined including Uiso. Water and ethanol solvent is heavily disordered and was modelled with partially occupied O and C atoms.

### Example 2

This Example provides synthesis, x-ray crystal structure, and molecular recognition properties of pillar[n]arene derivative P[6]AS which is referred to herein from time to time as Pillar[6]MaxQ, along with analogues P[5]AS and P[7]AS toward guests 11-28. This Example demonstrates ultratight binding affinity of P[5]AS and P[6]AS toward quaternary (di)ammonium ions, which supports their use for in vitro and in vivo non-covalent bioconjugation for imaging and delivery applications and as in vivo sequestration agents.

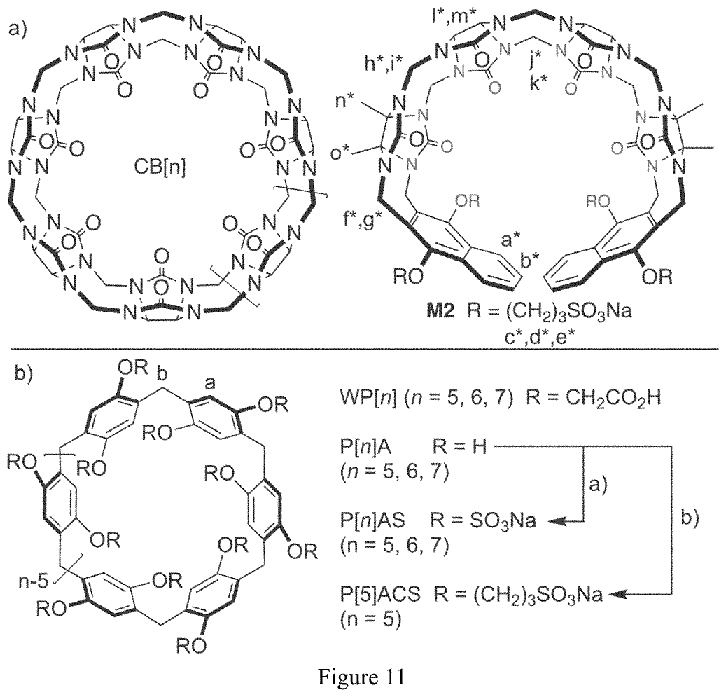

In more detail, it will be recognized by those skilled in the art that progress in the construction of supramolecular systems for biological (e.g., imaging and drug delivery) and chemical applications (e.g., sensing, catalysis, separations) depends critically on the availability of a library of building blocks that can be easily integrated into more complex and functional systems. Molecular containers-whether prepared by covalent bond forming reactions or by self-assembly processes-occupy a central space within the field. Some of the most popular molecular containers include cyclodextrins, calix[n]arenes, crown ethers, cyclophanes, coordination cages, molecular clips and tweezers, cucurbit[n]urils (CB[n]), and H-bonded capsules. Within this group, the CB[n] family (FIG. 11a) has proven particularly useful because they form tight CB[n].guest complexes in a selective and stimuli responsive manner which allows them to be used to create sensing ensembles, supramolecular polymers, molecular machines, for bioconjugation, as a non-covalent latching system, and for drug solubilization and delivery. Given the high binding affinity of acyclic CB[n] toward their best guests, acyclic CB[n] was developed (e.g., M2, FIG. 11a) as an in vivo sequestration agent for neuromuscular blockers and drugs of abuse. Most recently, the synthesis and molecular recognition properties of the pillar[n]arenes (FIG. 11b, e.g., WP[5] and WP[6]) in both organic and aqueous solutions have been extensively investigated and thoroughly reviewed with respect to their chemical and biological applications. Pillar[n]arenes represent a sweet spot for studies of molecular recognition in water in that they often display K_{d} values in the μM range and are more easily functionalized than CB[n]. This Example accordingly provides a description of preparation of pillar[n]arene sulfates (a.k.a. Pillar[n]MaxQ) that possess extreme binding affinity (Kₐ in μM range) toward quaternary diammonium ions in aqueous solution which make them particularly well suited as in vivo sequestration agents.

Contemplating the creation of new ultratight binding hosts based on pillar[n]arenes lead us to ponder the relevant structural features of CB[n] (FIG. 11a). The ultratight binding features of CB[n] have been traced to their highly electrostatically negative ureidyl C═O portals and the number and energetics of water molecules within the host cavity that are released upon binding (e.g. non-classical hydrophobic effect). By virtue of their double CH₂-linkers, CB[n] possess no free rotors, cannot undergo self-complexation, and are therefore highly pre-organized hosts. The present disclosure relates to replicating these structural features in the pillar[n]arene family by rational molecular design. Although anionic Water soluble Pillararenes (e.g. WP[5] and WP[6]) are known they contain CH₂-linkers between the aromatic ring and the anionic functional groups (e.g., carboxylate, sulfonate, phosphonate). The disclosure includes removing the CH₂-linkers and changing to the highly acidic sulfate functional group to provide a higher negative charge density around the mouth of the cavity. Simultaneously, the addition of two sulfate groups per phenylene group were envisioned to electrostatically minimize the known possibility of the phenylene groups leaning into their own cavity.

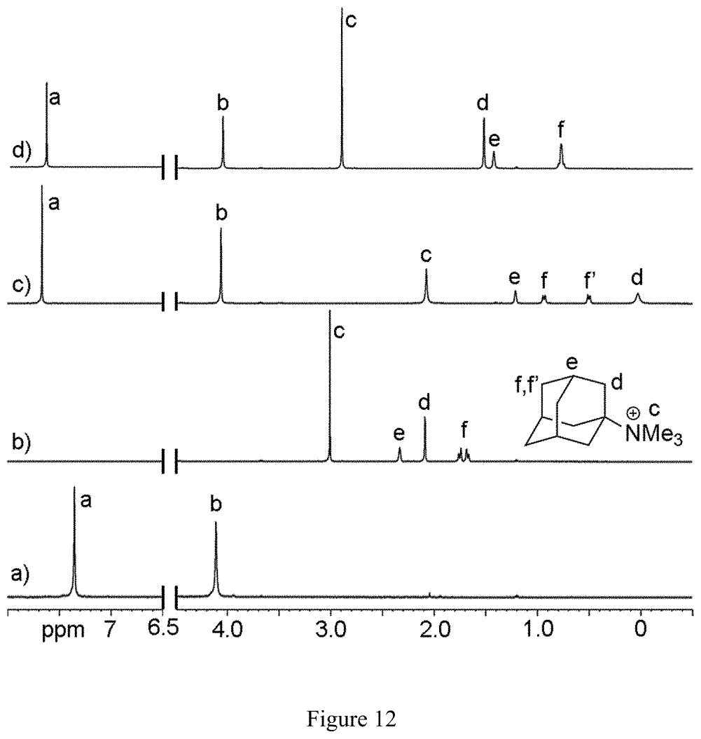

FIG. 11 shows the synthesis of P[5]AS-P[7]AS. The parent hydroxylated pillararenes (P[5]A-P[7]A) were prepared according to the literature procedures. Subsequently, P[5]A-P[7]A were individually reacted with pyridine.SO₃ in pyridine at 90° C. to deliver P[5]AS-P[7]AS in 70, 66, and 46% yield, respectively. To gain insight into the role of the CH₂-linkers, P[5]ACS was prepared as a control compound in poor yield (8%) by the reaction of P[5]A with propane sultone and NaOH in acetone. Lastly, known hosts WP[5] and WP[6] were prepared by methods known in the art as additional comparators. All new compounds were fully characterized by ¹H and ¹³C NMR, IR, and high resolution electrospray ionization mass spectrometry. It is known that pillar[6]arenes may exist in five different conformational forms due to rotation around the phenylene units. FIG. 12a shows the ¹H NMR recorded for P[6]AS in D₂O at room temperature which consists of two relatively sharp singlets. This indicates that P[6]AS is either locked into the depicted C₆-symmetric structure or the phenylene units are rotating rapidly on the chemical shift timescale. Based on the host.guest experiments, it can be concluded that rotation of the OSO₃⁻ groups through the annulus of P[6]AS pillararene is fast.

The inherent aqueous solubility of the two most potent hosts (P[5]AS: 100 mM; P[6]AS: 20 mM; vide infra) were measured by integrating the ¹H NMR resonances for a solution of host against the methyl resonance for sodium 3-(trimethylsilyl)propionate-2,2,3,3-d₄ as internal standard of known concentration. Before proceeding to investigate the host.guest properties of the new hosts, we performed dilution experiments monitored by ¹H NMR spectroscopy to quantify their intermolecular self-association. The spectra were recorded for P[5]AS and P[6]AS as a function of concentration (P[5]AS: 20-0.1 mM; P[6]AS: 20-0.1 mM) used to calculate Ks values (P[5]AS: 19.7 M⁻¹; P[6]AS: 16.2 M⁻¹) by using a standard 2-fold self-association model. These Ks values ensure that the hosts remain monomeric at the mM concentrations used in the NMR and ITC experiments described below in this Example. Crystals of both P[6]AS and P[5]ACS were obtained and their structures as solved by x-ray diffraction measurements (FIG. 13, CCDC 1996177 and CCDC 1996179). FIG. 13d shows the structure of one molecule of P[5]ACS in the crystal. As is commonly seen in pillararene crystal structures, the phenylene rings are oriented roughly perpendicular to the mean plane of the macrocycle and the substituents serve to deepen the cavity. The S . . . S distances between sulfonates attached to a single phenylene ring ranges from 14.785-15.467 Å. FIG. 13a shows the structure of a single molecule of P[6]AS in the crystal. In contrast to P[5]ACS, P[6]AS adopts an unusual conformation in which alternating phenylene units lean slightly into the cavity on opposite faces of the macrocycle in a geometry reminiscent of cyclotriveratrylenes. The leaning of phenylenes from perpendicular measures 35-38 degrees. Interestingly, the OSO₃⁻ groups do not lie in the mean plane of the phenylene units and instead are alternately displayed above and below the plane. This leaning and alternation results in the placement of the twelve OSO₃⁻ groups roughly at the corners and edges of a triangular antiprism of side length 11.130 Å and height 6.714 Å. Accordingly, P[6]AS packs a remarkably high charge density of ˜12 within a small volume (CPK molecular volume (MMFF) of P[6]AS=1173 Å³). The influence of the Na⁺ counterions on the observed conformation of P[6]AS is unclear. The molecules of P[6]AS pack into a hexagonal array in the xy-plane as shown in FIG. 13b; the OSO₃⁻ subunits are extensively bridged by coordinating Na⁺ ions. These hexagonally packed sheets of P[6]AS pack along the z-axis in register with each other such that the P[6]AS units define a tube (FIG. 13c). The packing of P[5]ACS also displays stacked sheets held together by networks of bridging Na⁺ ions.

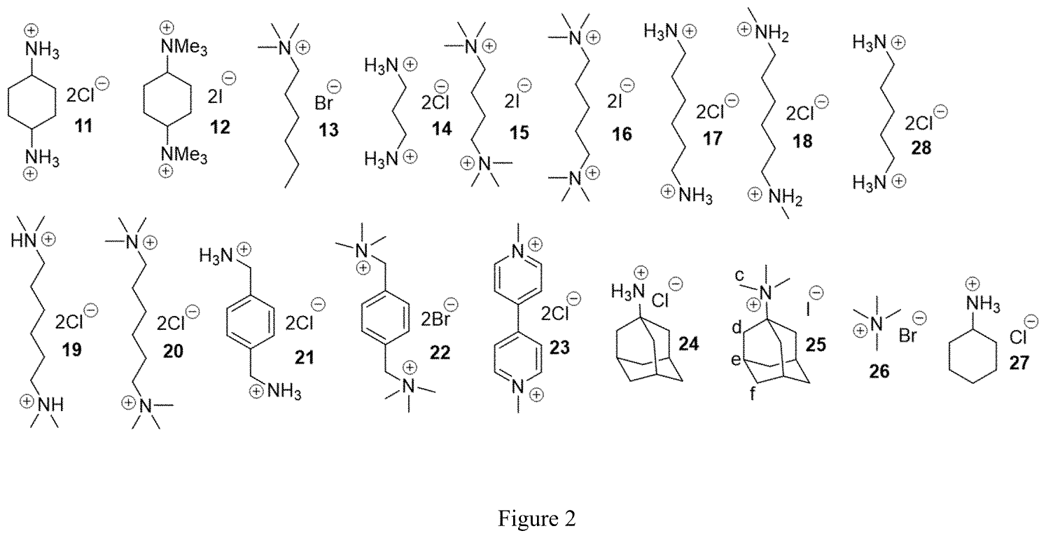

FIG. 12a shows two singlets for P[6]AS alone and a single set of sharp resonances for the P[6]AS.25 complex (FIG. 12c). The substantial upfield shifting observed for the resonances of guest 25 confirm its inclusion in the cavity of P[6]AS. At a 1:2 P[6]AS:25 ratio, the resonances for guest 25 shift back toward those of free 25 which indicates that guest exchange occurs rapidly on the chemical shift timescale. Similar investigations were performed for different combinations of hosts and guests from FIGS. 2-4 and in many cases the situation was more complex. For example, in many cases the resonances for the aryl H-atoms (Hₐ) become broadened or split into many distinct sharp resonances upon mixing with one equivalent of guest. Of course, it is well known that pillar[n] arenes possess several different lower symmetry conformations (n=5:4 conformers; n=6:5 conformers) which would be expected to give rise to broadened or additional resonances as was observed in a guest dependent manner. For hosts P[6]AS and P[7]AS, upfield shifting of guest resonances was observed upon binding indicating cavity binding of the guest hydrophobic moiety. For P[5]AS, narrower guests (e.g., 21 and 23) bind inside the cavity as indicated by upfield changes in chemical shift, but wider guests (e.g., 12 and 25) show upfield shifts of NMe₃⁺ groups rather than their hydrophobic moieties, which indicate ⁺NMe₃ binding near the portals. ITC measurements (vide infra) indicate that 12 and 25 bind to P[5]AS with 1:2 host:guest stoichiometry.

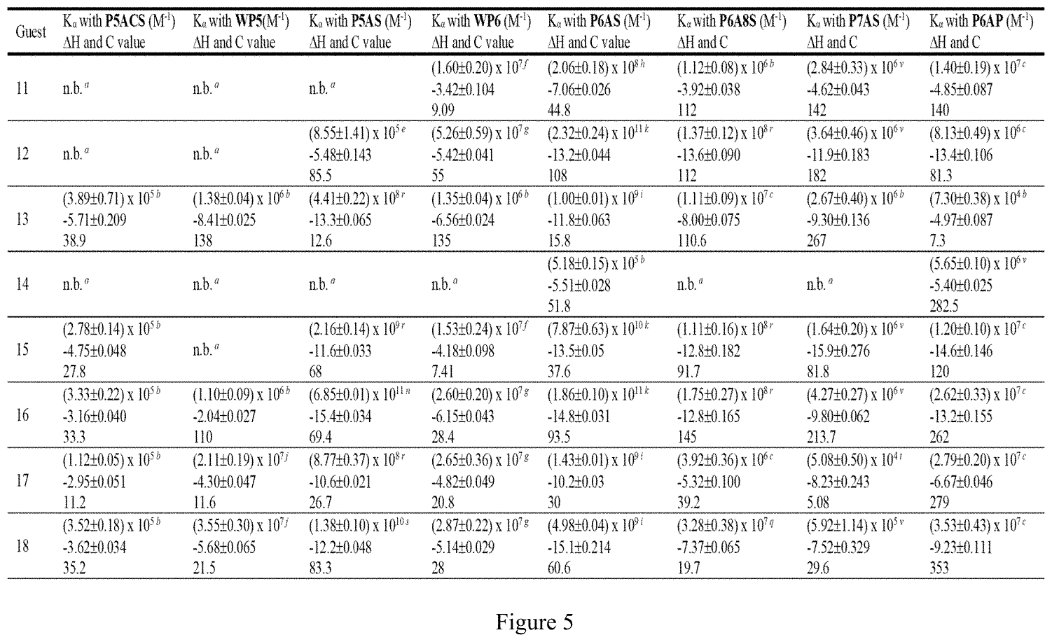

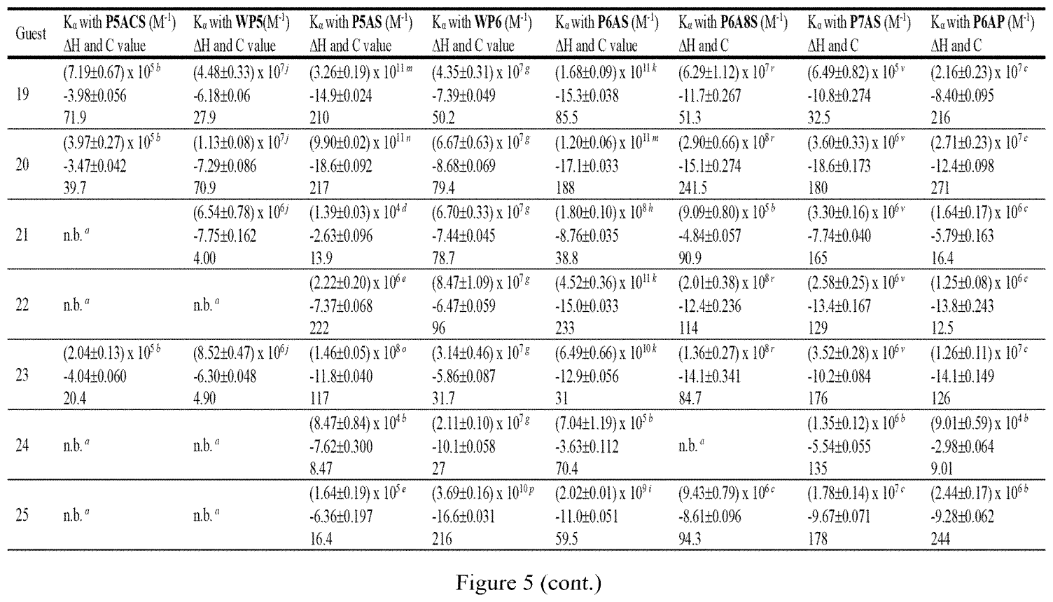

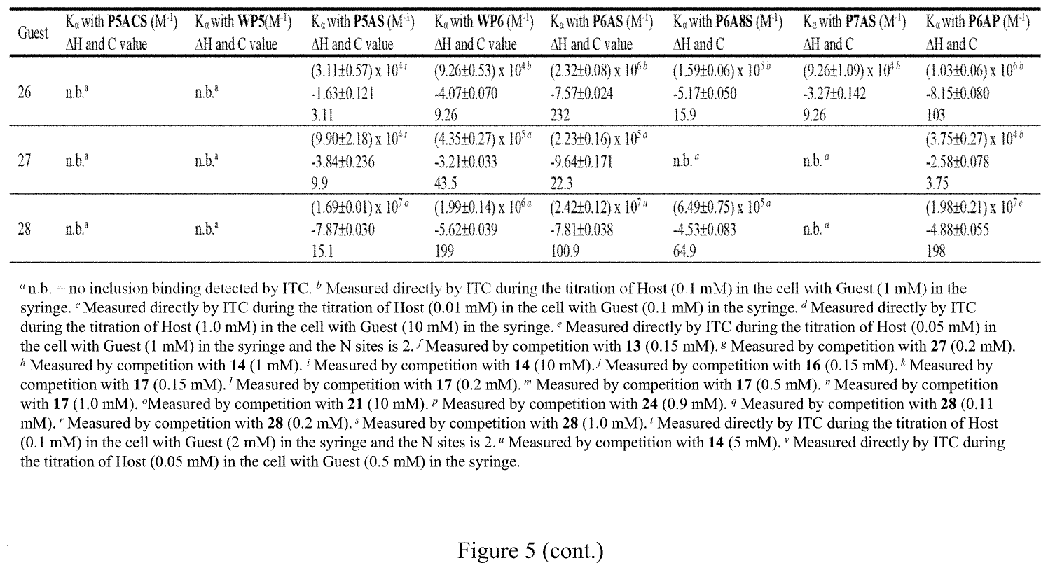

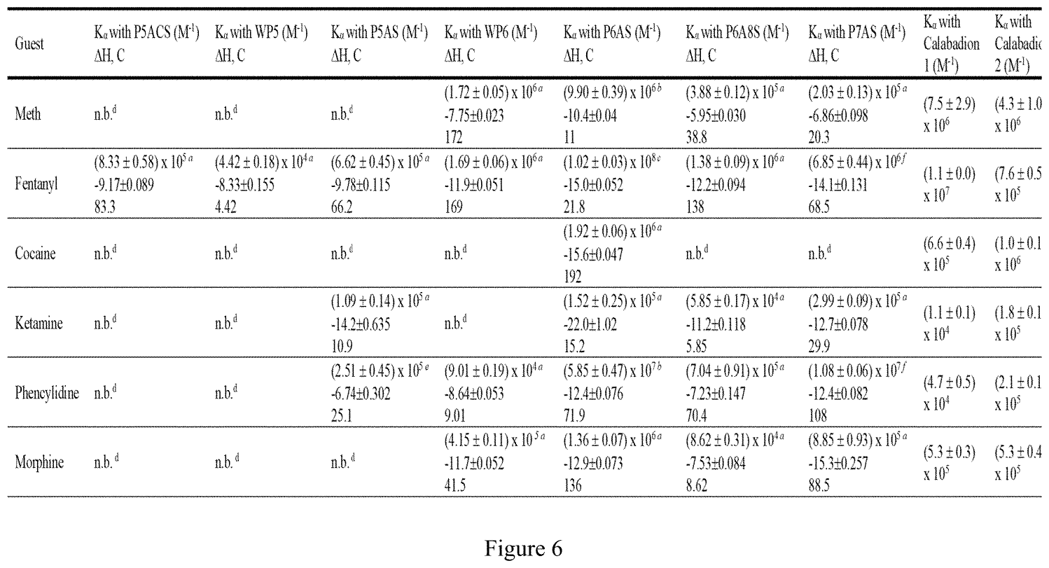

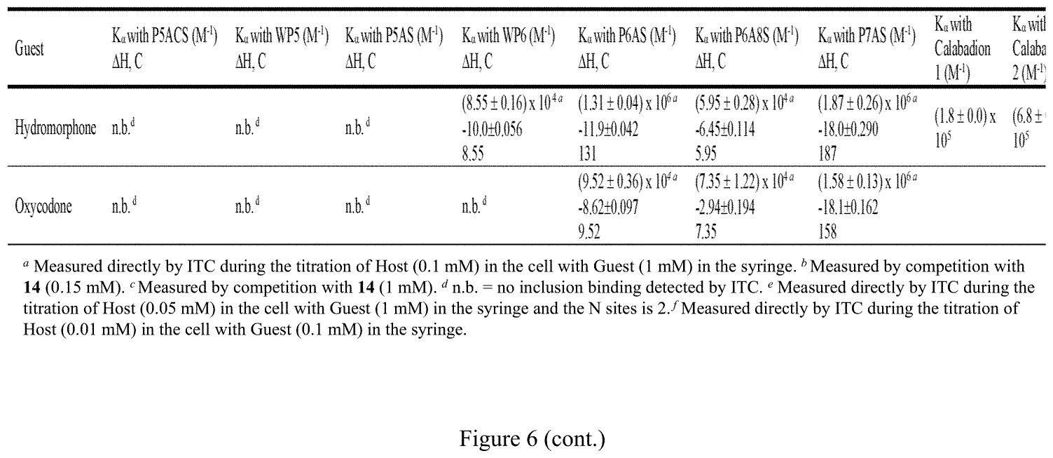

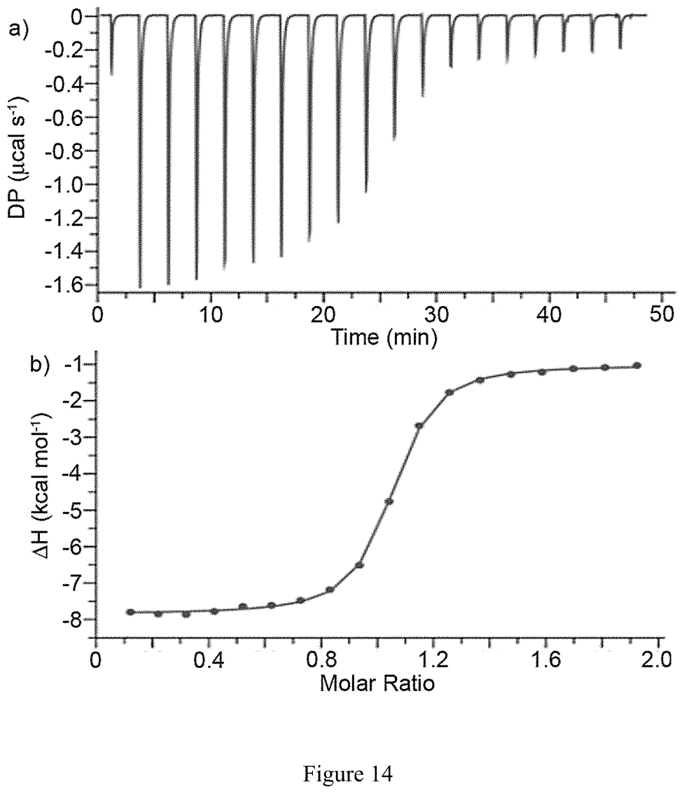

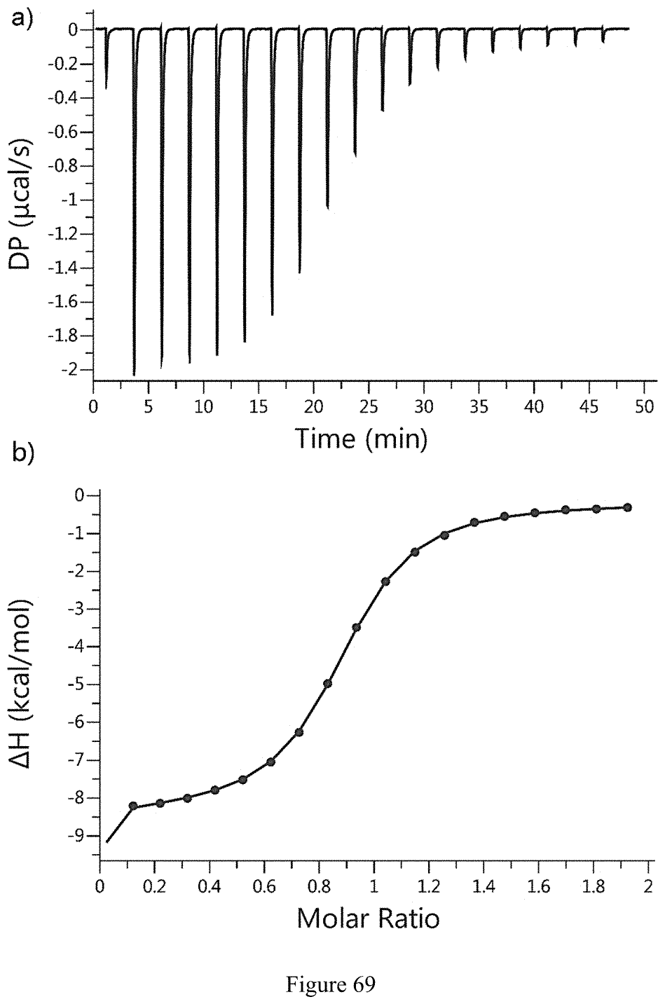

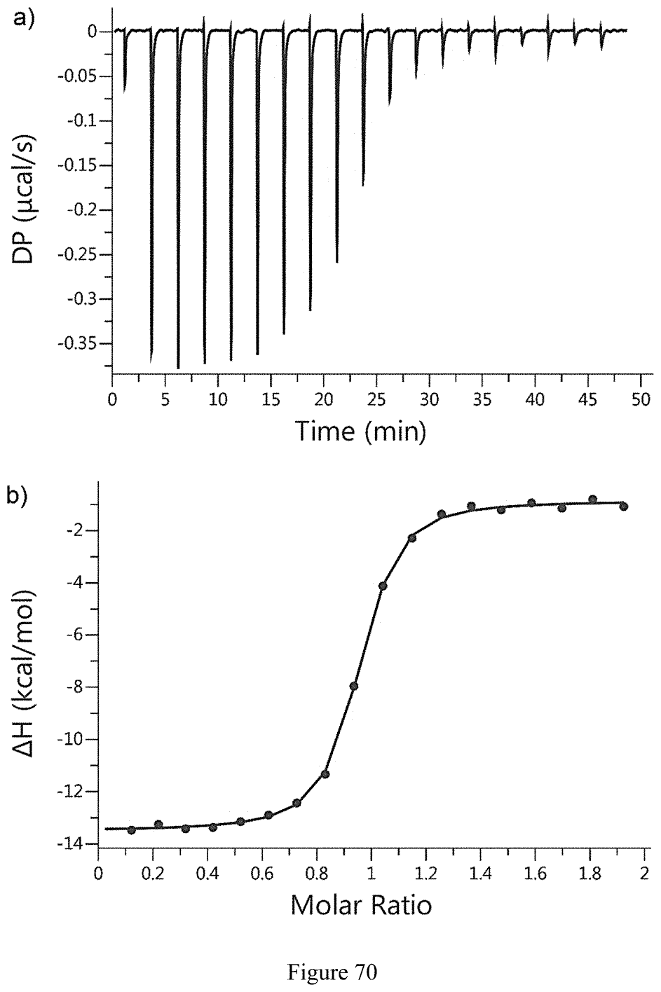

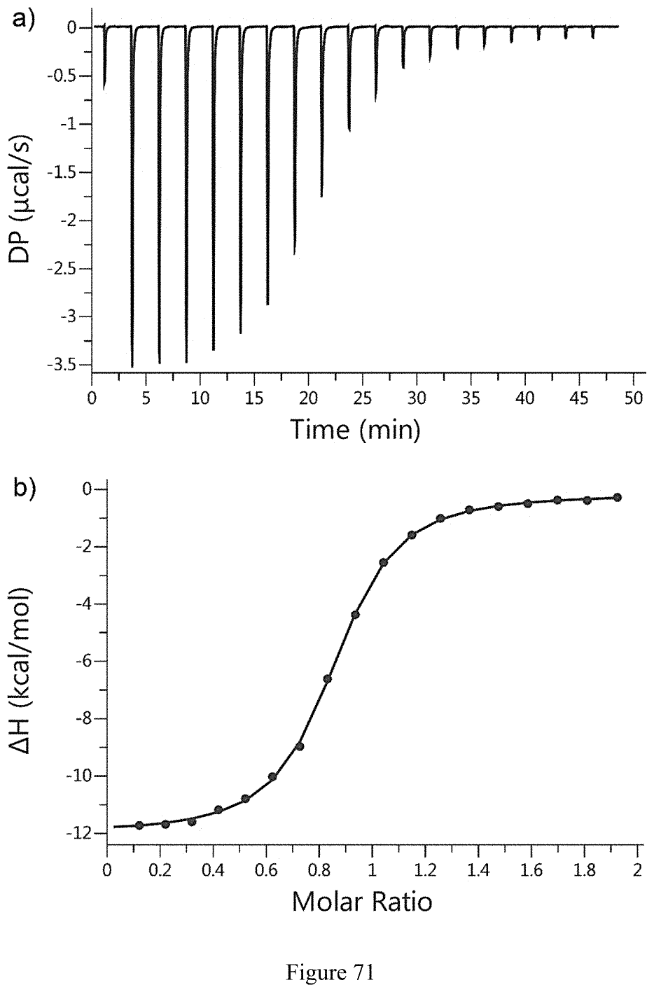

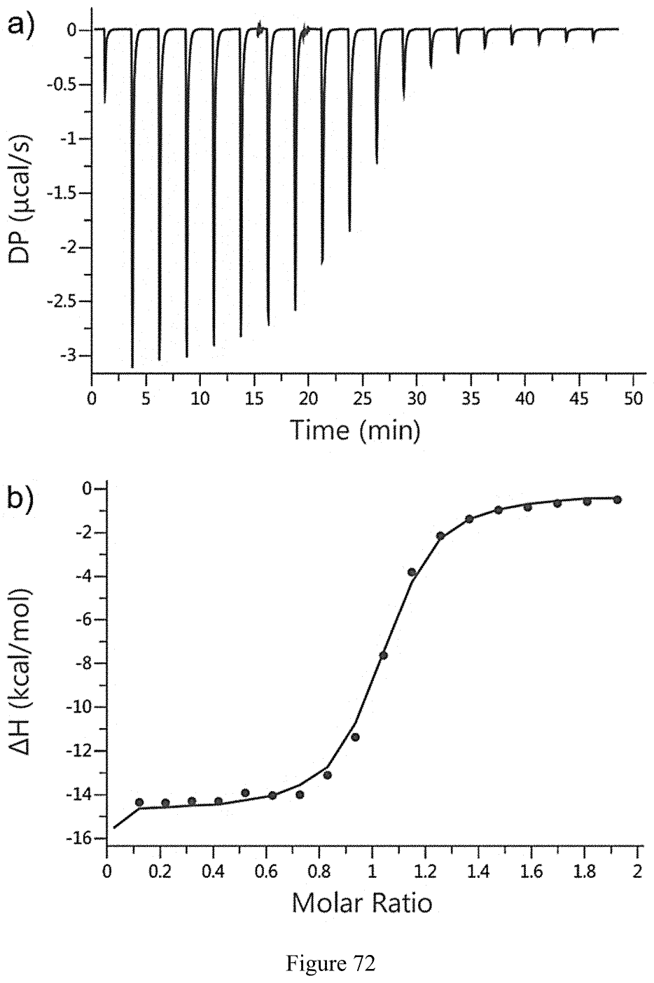

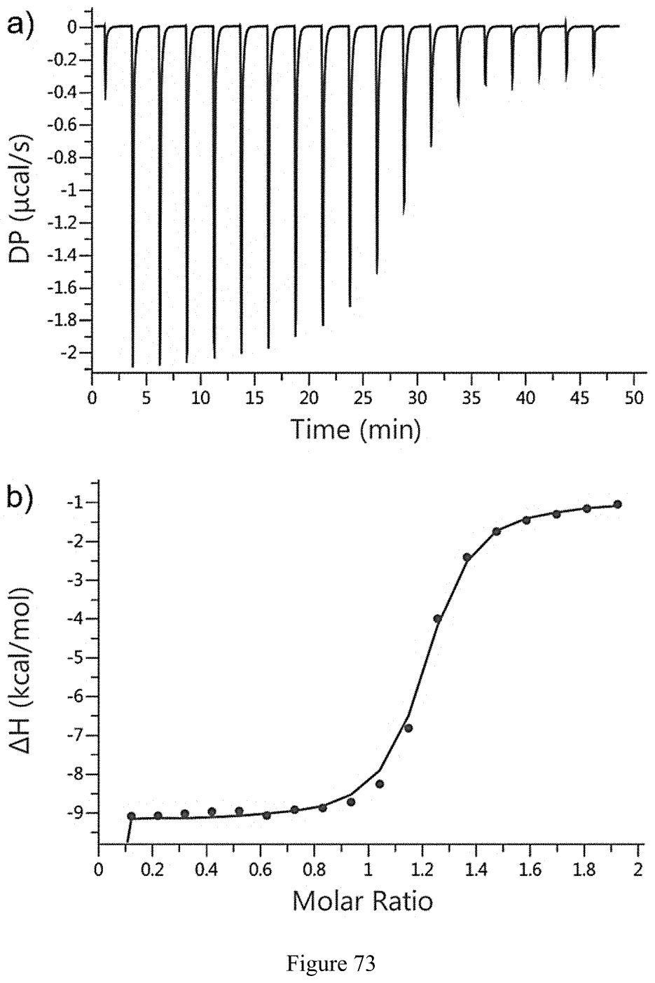

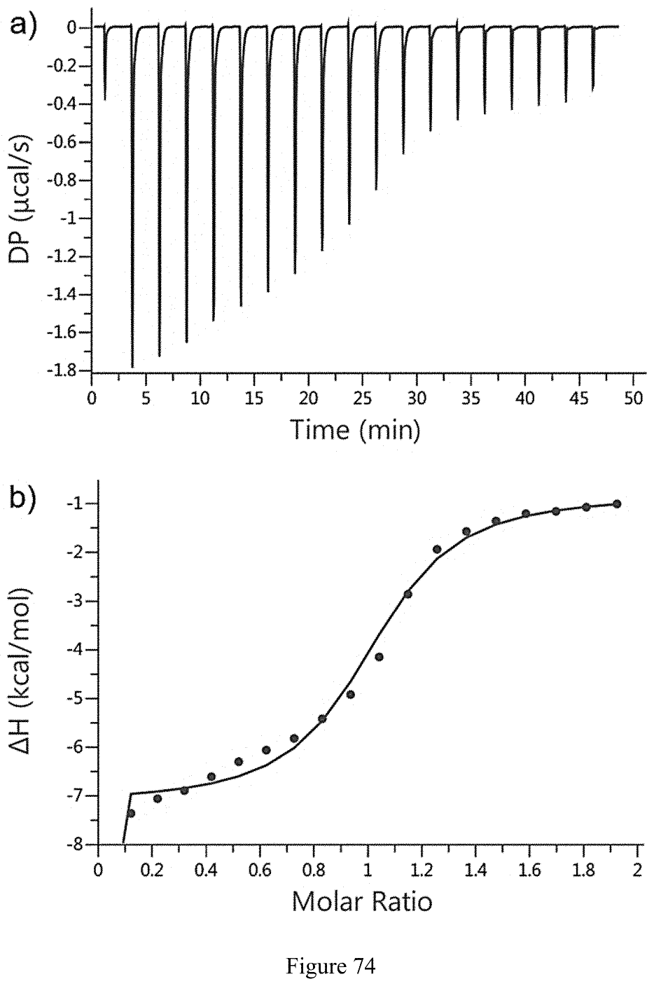

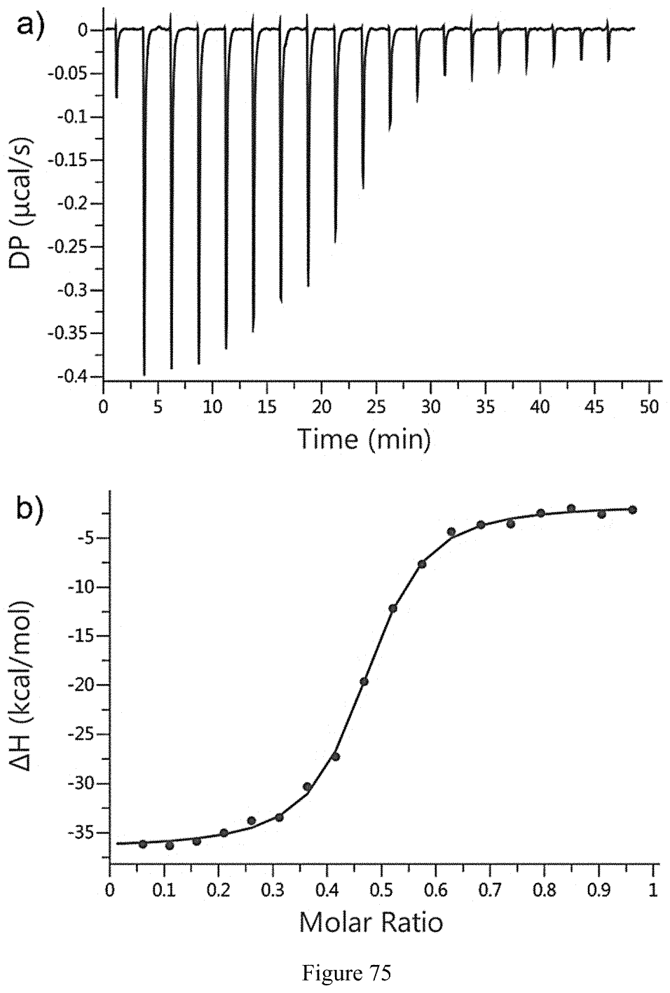

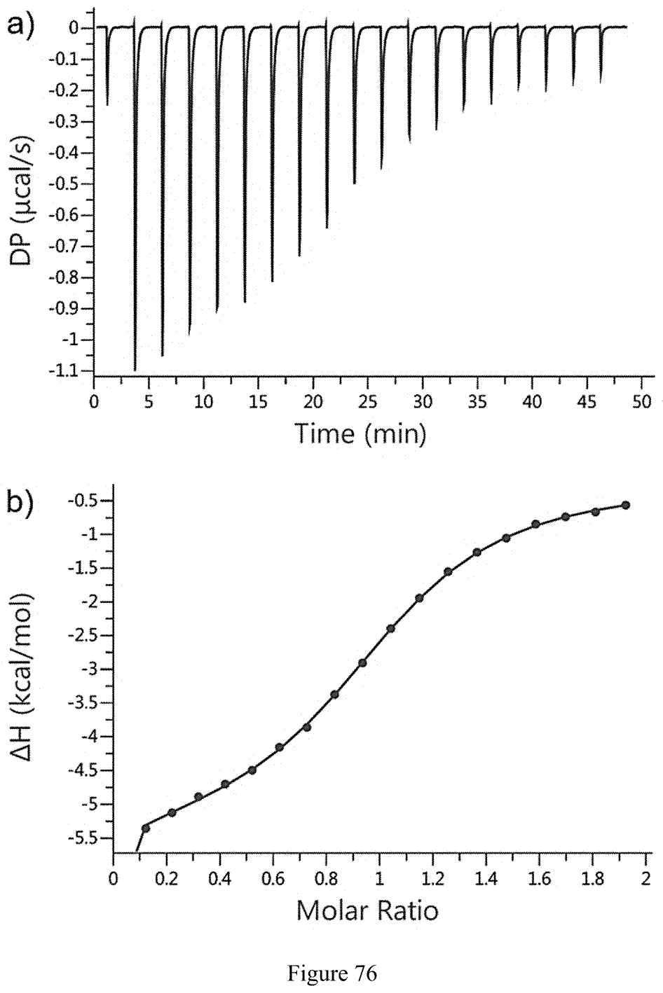

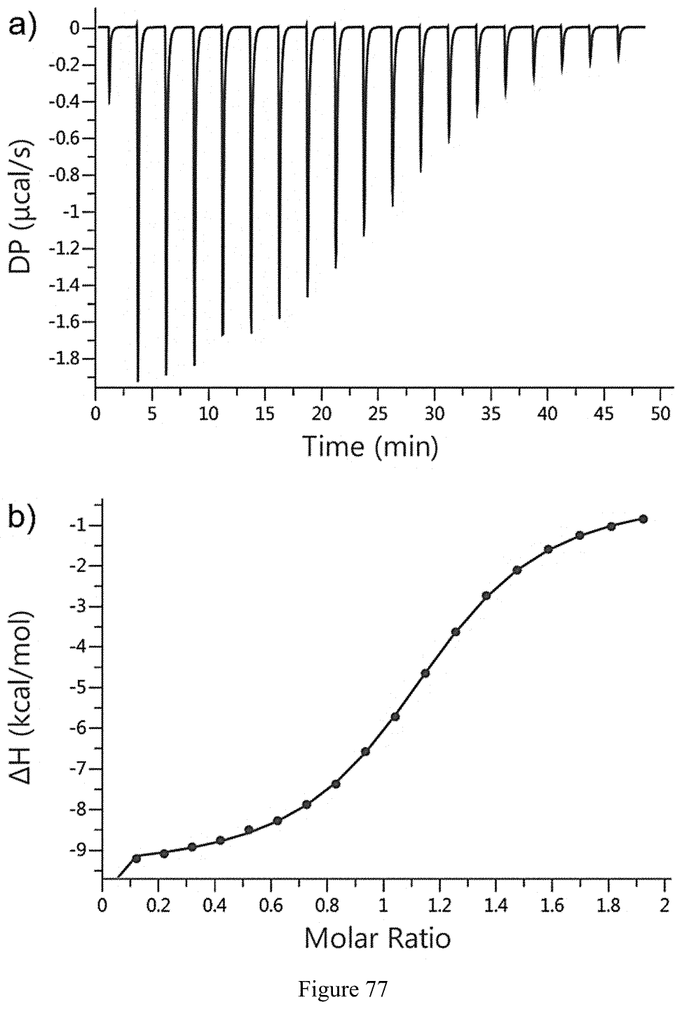

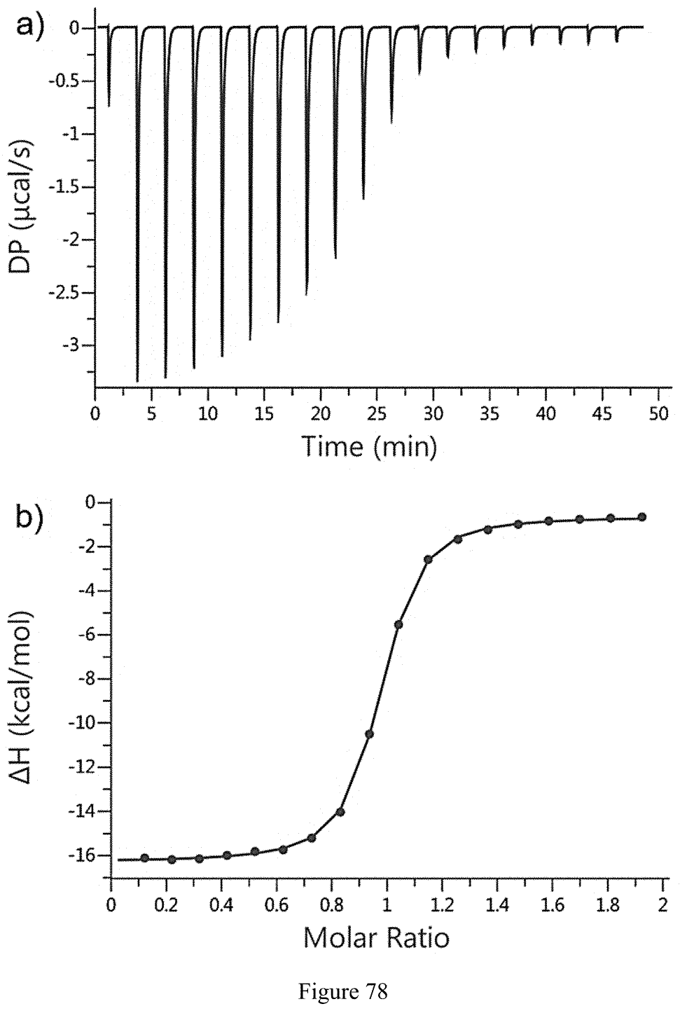

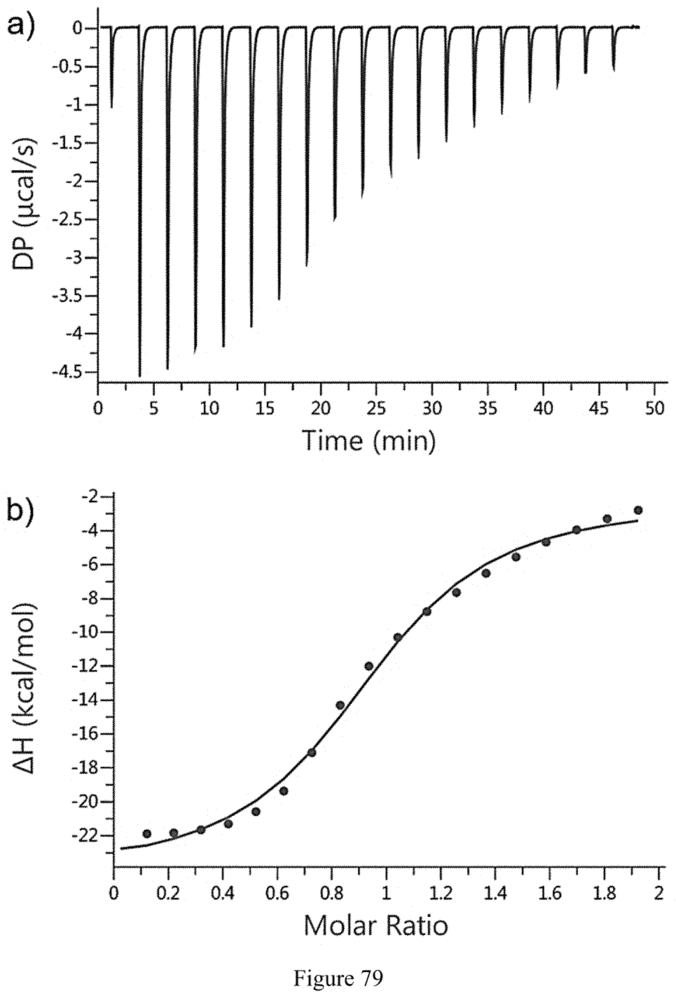

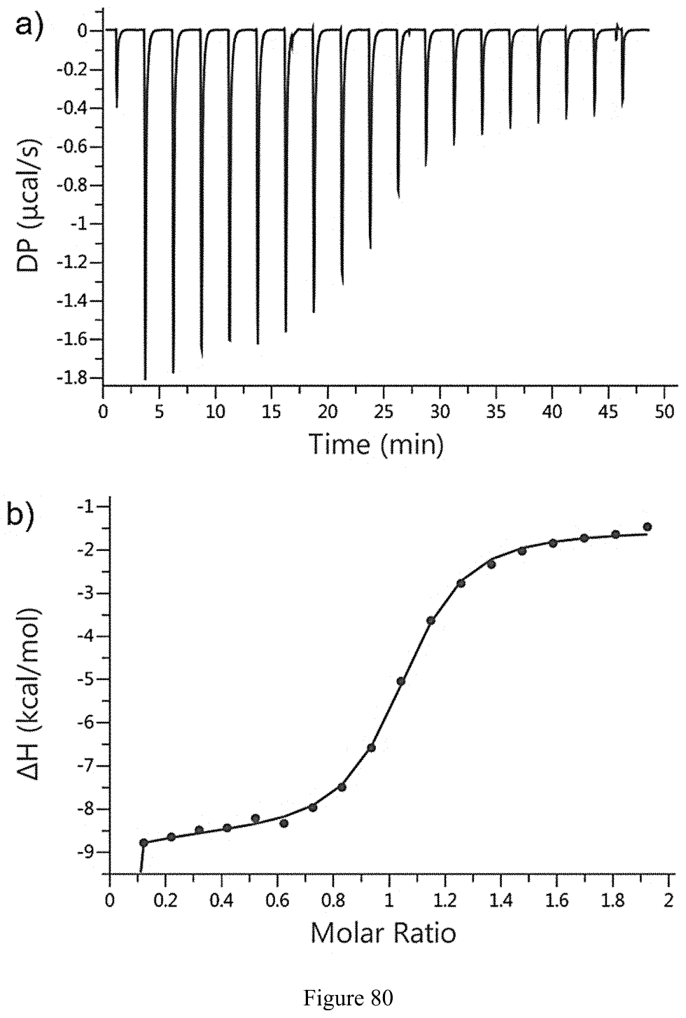

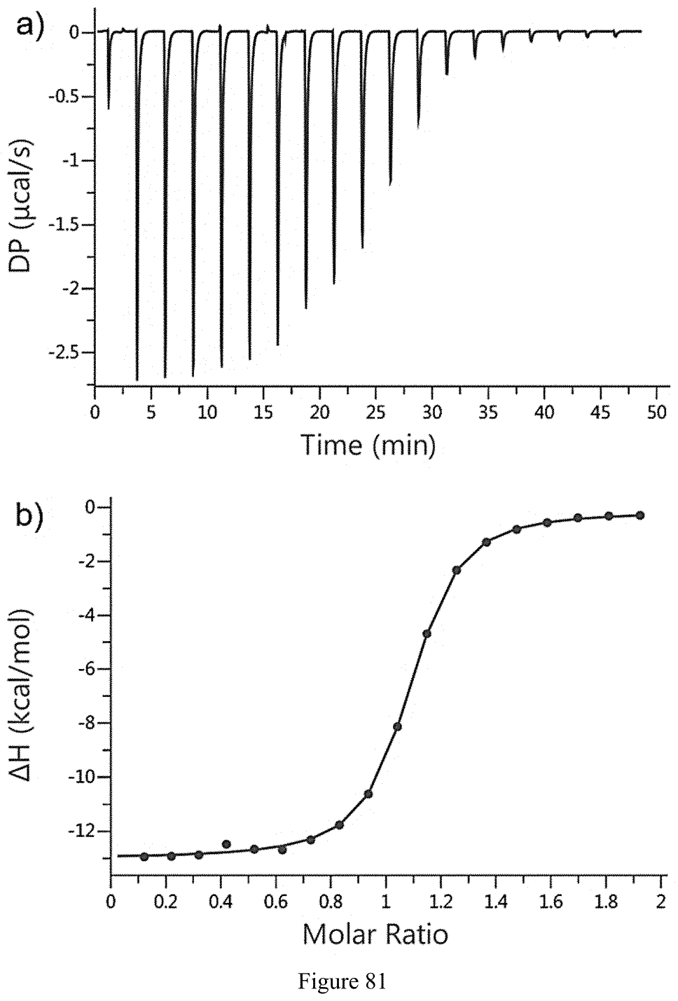

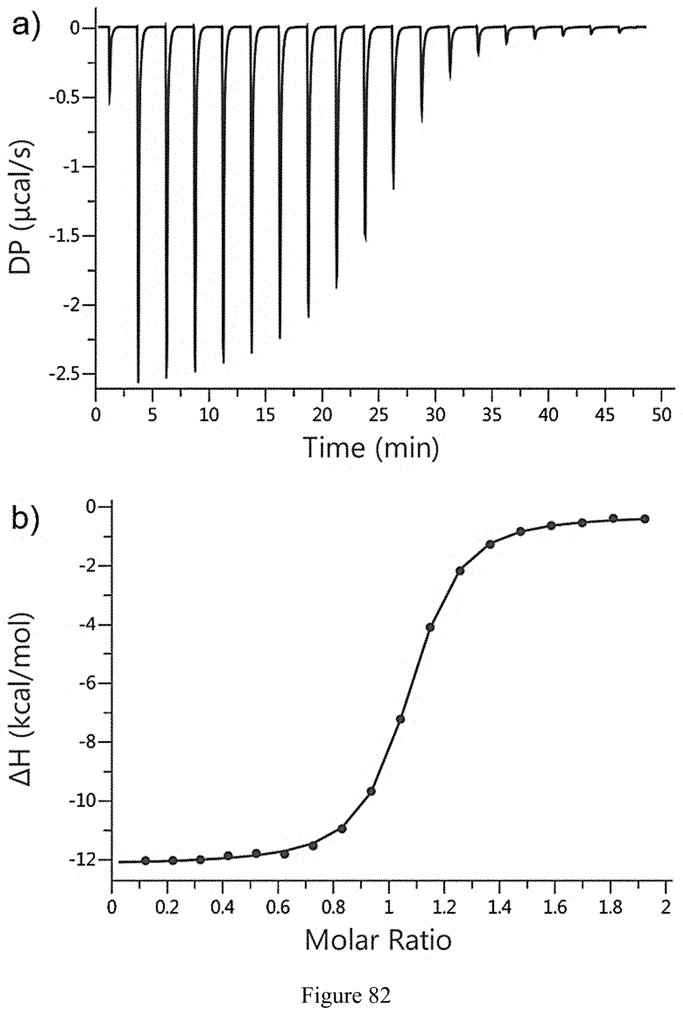

Initially, the molecular recognition properties of the new hosts toward guests 11-28 (FIG. 2) by were investigated by ¹H NMR spectroscopy. Compounds 11-28 were selected because they feature different numbers of charged groups (one or two), length of hydrophobic residue, width of hydrophobic residue, and degree of ammonium ion substitution (1°, 2°, 3°, 4°) to assess the preferences of the new hosts. FIG. 12 shows the ¹H NMR spectra recorded for P[6]AS, 25, and 1:1 and 1:2 mixtures of P[6]AS and 25 which is a particularly well resolved example. Next, the strength of the binding interactions between the various hosts and guests was quantified. Given the complexity of the ¹H NMR spectra and the observed tight binding (vide infra) we used isothermal titration calorimetry (ITC). For most complexes, we performed direct titration of host in the cell with guest in the syringe. FIGS. 5-7 shows the thermodynamic parameters determined by these direct ITC titrations and the representative experimental data is shown in FIGS. 69-83. Direct titrations were inappropriate for the tighter host.guest complexes where Kₐ values exceeded 4×10⁷ M⁻¹ where the c-value exceeded the recommended range even when working at [host]=10 μM. In these cases, competition ITC experiments were employed where a mixture of host and an excess of a weaker binding guest in the cell was titrated with a stronger binding guest in the syringe. In these ITC competition experiments, the ΔH and Kₐ values for the weaker host.guest complex are determined independently and used as inputs for the competitive ITC titrations. FIG. 14a shows the titration of a mixture of P[6]AS and weaker binding guest 17 in the cell with the stronger binding guest 20 in the syringe. Fitting of the data (FIG. 14b) to a competitive binding model allowed the extraction of the thermodynamic parameters for P[6]AS.20 (Kₐ=(1.20±0.06)×10¹¹ M⁻¹; ΔH=−17.1±0.033 kcal mol-1). FIGS. 5-7 reports the results of competitive ITC titrations for the tighter host.guest complexes.

The extensive dataset presented in FIGS. 5-7 allows a thorough discussion of the binding preferences of the new hosts in comparison to the previously known WP[5] and WP[6]. All of the complexes are driven by favorable ΔH values which suggests these complexes benefit from the non-classical hydrophobic effect as planned. First, it is noted that P[5]ACS with its (CH₂)₃-linkers binds ≈10¹-10²-fold more weakly toward alkanediammonium ions 16-20 than observed for WP[5], which may be a consequence of the linkers partially occluding the host cavity or the longer linker to the anionic SO₃⁻ group diminishing electrostatic interactions. Furthermore, P[5]ACS and WP[5] display little selectivity in binding based on the degree of methylation of the diammonium ion (e.g. 1°: 17, 2°: 18; 3°: 19; 4°: 20). In contrast, P[5]AS is a superior host toward diammonium ions than WP[5] (e.g. 17: 41-fold; 18: 390-fold; 19: 7300-fold; 20: 88000-fold). P[5]AS displays increasing binding affinity as the degree of methylation of the N-atoms of the guest are increased. Accordingly, this class of hosts was dubbed as Pillar[n]MaxQ to denote their generally superior binding affinity and selectivity toward quaternary ammonium ions. A comparison of the binding affinities of P[5]AS toward different length quaternary diammonium ions (e.g. 15, 16, 20) shows that the C₄-diammonium ion binds 317-458-fold more weakly than the C₅- and C₆-analogues presumably due to better matching of the N . . . N to ⁻O₃S . . . SO₃⁻ distance and the increased hydrophobicity of the C₆-hydrophobic residue. Quite interestingly, a comparison of the affinity of P[5]AS toward mono quaternary guest 13 (4.41×10⁸ M⁻¹) and bis quaternary guest 20 (9.90×10¹¹ M⁻¹) reveals the importance of electrostatic interactions in the recognition process. All of these narrow guests form 1:1 P[5]AS.guest complexes. In contrast, the ITC results reveal that wider guests (e.g. 12, 25, 27, cis, roc, vec, pan) cannot form inclusion complexes with P[5]AS and instead form 1:2 P[5]AS:guest complexes at the portals. The ITC titrations of P[5]AS with this subset of guests fit well to a 1:1 binding model with N=2, and therefore the Kₐ values reported in FIGS. 5-7 have M⁻¹ units and refer to each of the two independent binding events. The Kₐ value of P[5]AS toward Me₄N⁺ (P[5]AS.26; Kₐ=3.11×10⁴ M⁻¹) reveals that each quaternary ammonium ion head group makes a large contribution toward the observed ultrahigh affinity of P[5]AS toward (bis)quaternary ammonium ions (e.g., 20).

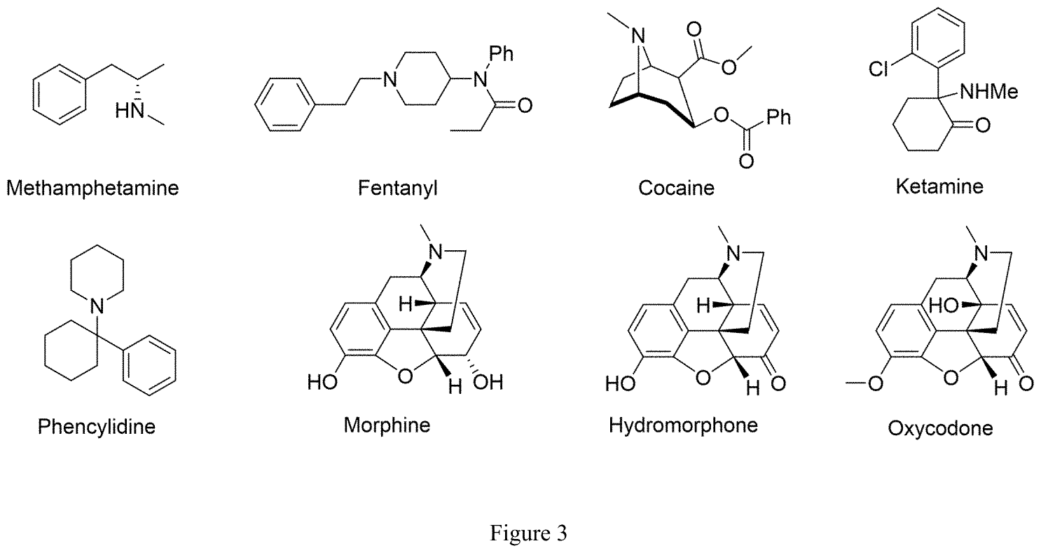

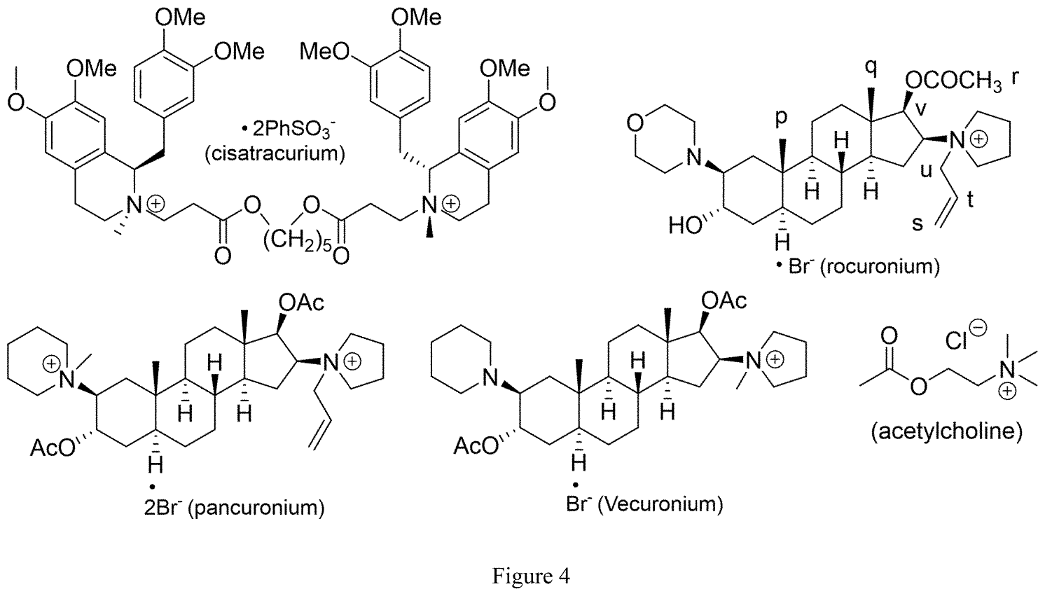

FIGS. 5-7 show the binding constants (Kₐ, M⁻¹) and thermodynamic parameter (ΔH, kcal mol⁻¹) for various hosts and guests 11-28, the neuromuscular blocking agents are shown in FIG. 4, and drugs of abuse are show in FIG. 3 and FIG. 68. Conditions: H₂O, 20 mM NaH₂PO₄ buffer, pH 7.4, 298K.—not measured. n.b.=no heat change detected by ITC. a Measured by direct ITC titration with [host]≥10 μM. ^{b} Measured by competitive ITC titration with 13. ^{c} Measured by competitive ITC titration with 14. ^{d}Measured by competitive ITC titration with 16. ^{e} Measured by competitive ITC titration with 17. ^{f} Measured by competitive ITC titration with 21. ^{g} Measured by competitive ITC titration with 24. ^{h} Measured by competitive ITC titration with 27. ⁱ Measured by competitive ITC titration with 28. ^{j} 1:2 host:guest complex. ^{k} 2:1 host:guest complex.

Related comparisons can be made between hosts WP[6] and P[6]AS which exhibit 1:1 host: guest complexation toward all the guests used in this study. For example, P[6]AS is the superior host toward 20 out of the 23 guests studied with exceptions including 1° ammonium ions 24 and 27. Similar to P[5]AS, P[6]AS is highly selective based on guest length (e.g. 14 vs 28 vs 17; 15 vs 16 vs 20) and on the degree of methylation of the diammonium ion (e.g. 17 vs 20; Kₐ=1.43×10⁹ vs 1.20×10¹¹ M⁻¹). Interestingly, the binding affinity of P[6]AS toward Me₄N⁺(26, Kₐ=2.32×10⁶ M⁻¹) is 75-fold stronger than P[5]AS which suggests P[6]AS should be regarded as a powerful host for quaternary ammonium ions. In fact the Kₐ values of P[6]AS toward the guest panel lie in the single digit μM to 1 μM range which places P[6]AS squarely alongside CB[n] as one of the highest affinity synthetic host.guest systems in water although the balance between complexation driving forces (e.g. electrostatic versus hydrophobic effect) obviously differs. Finally, FIGS. 5-7 present the binding affinities of P[7]AS toward the panel of guests (11-28). In this case, comparison with the water soluble pillararene analogue (WP[7]) could not be performed since access to it failed. Regardless, a perusal of FIGS. 5-7 reveal that P[7]AS is a significantly less potent receptor toward the guest panel than P[6]AS with the exception of the primary ammonium ion 24. Although the reasons for the relatively poor performance of P[7]AS are not established, without intending to be bound by any particular theory, it was surmised the reasons may parallel those of the CB[n] host family where the size of the electrostatically negative portals and the energetics and number of bound waters in the host cavity play important roles.

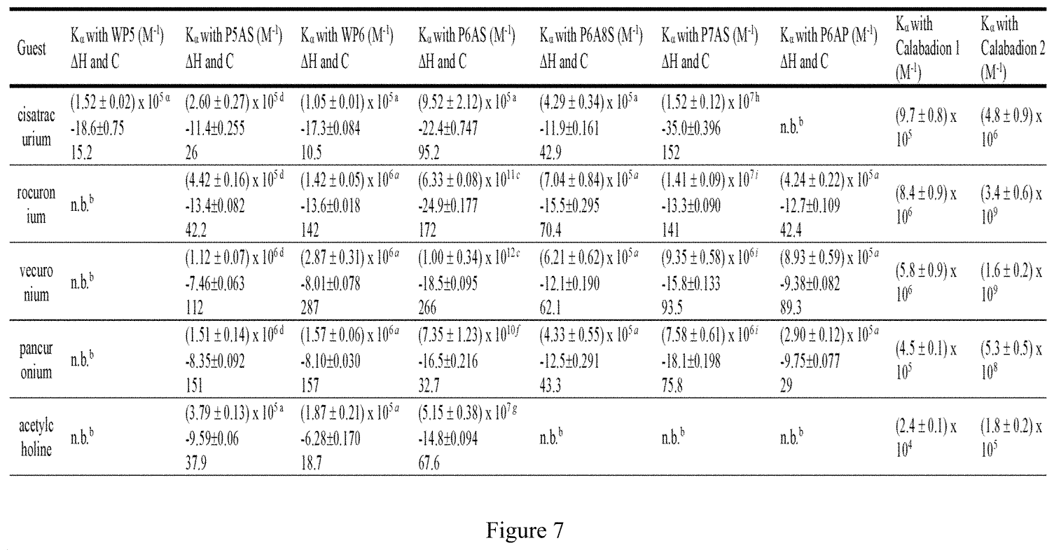

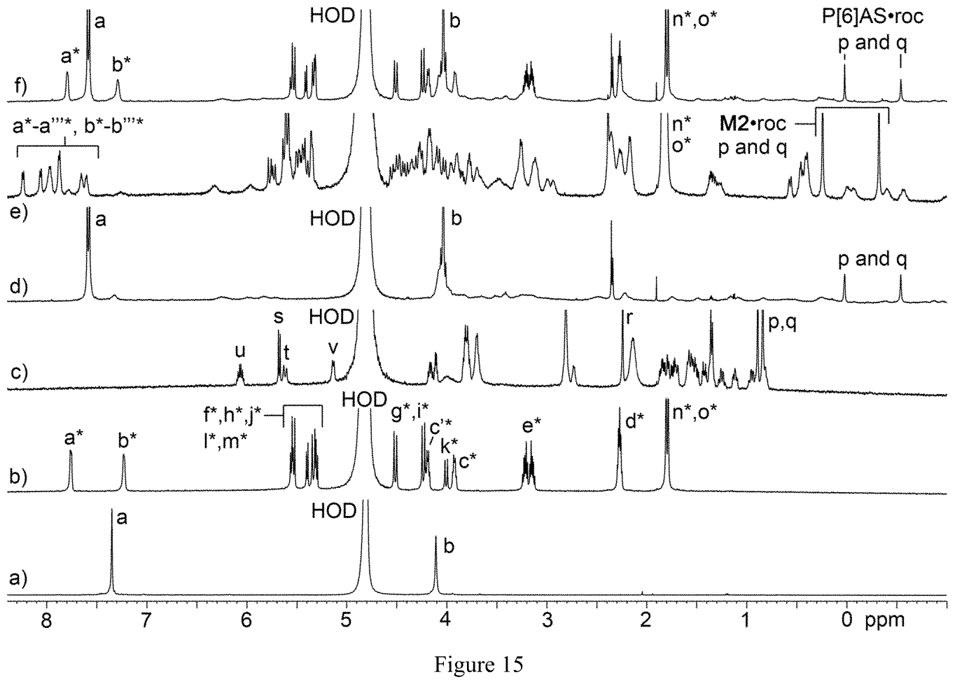

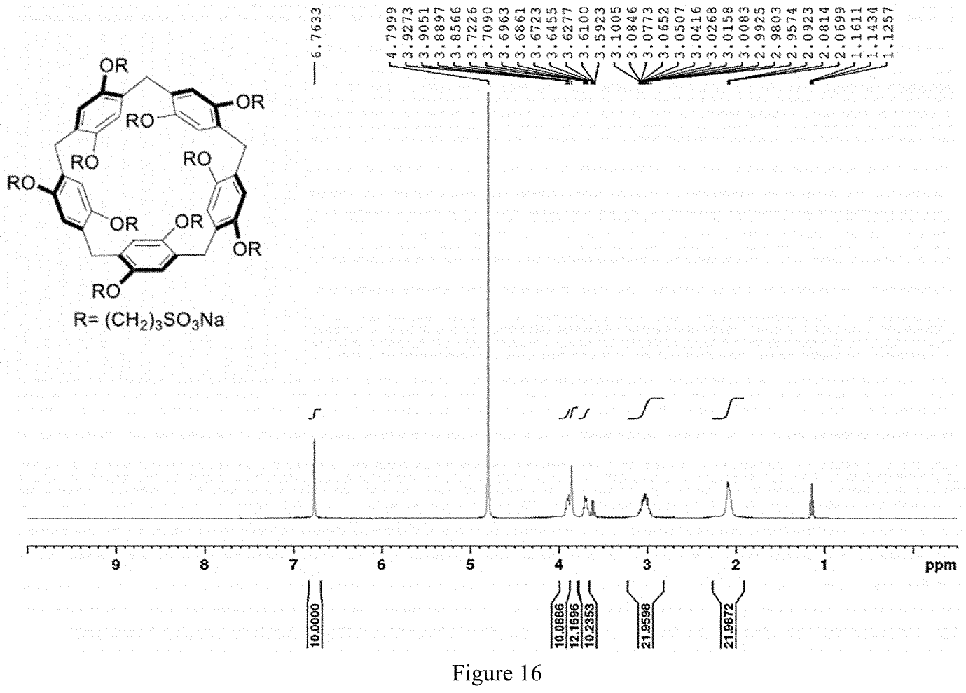

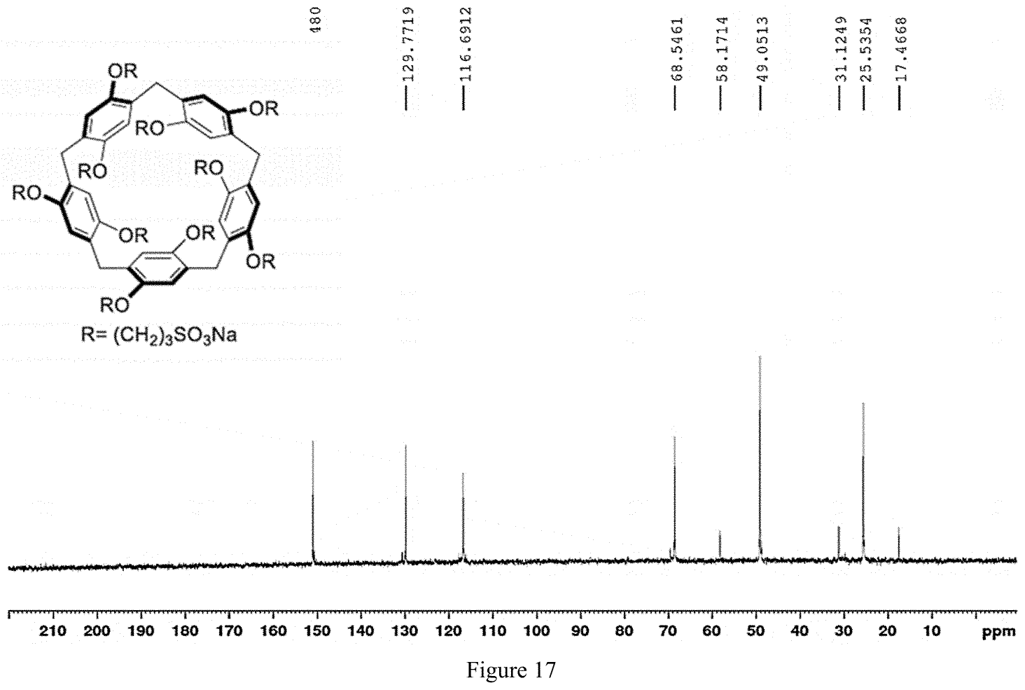

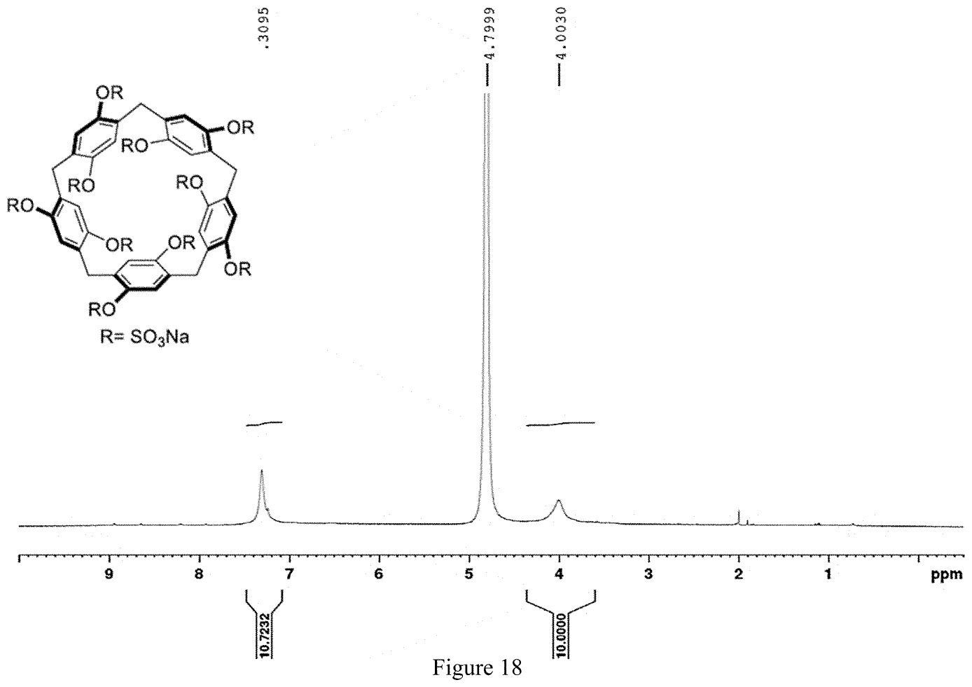

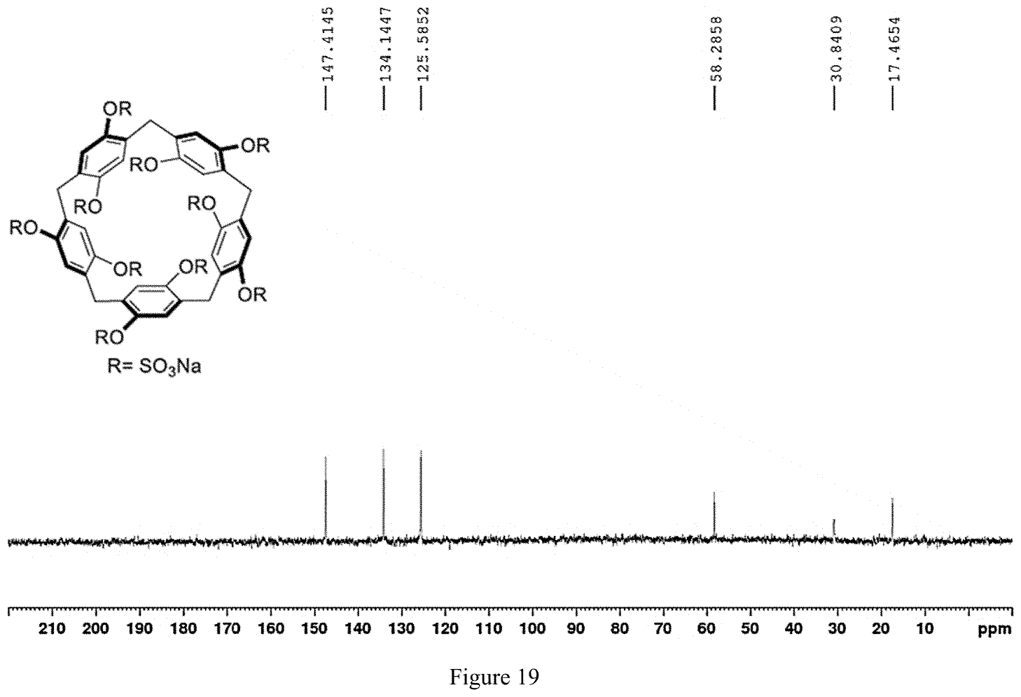

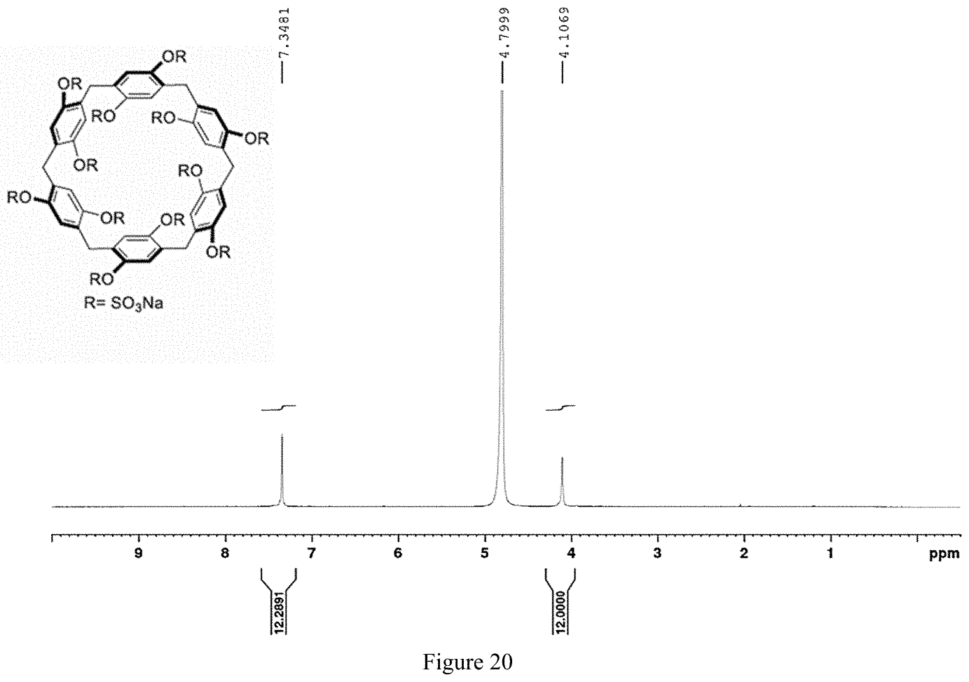

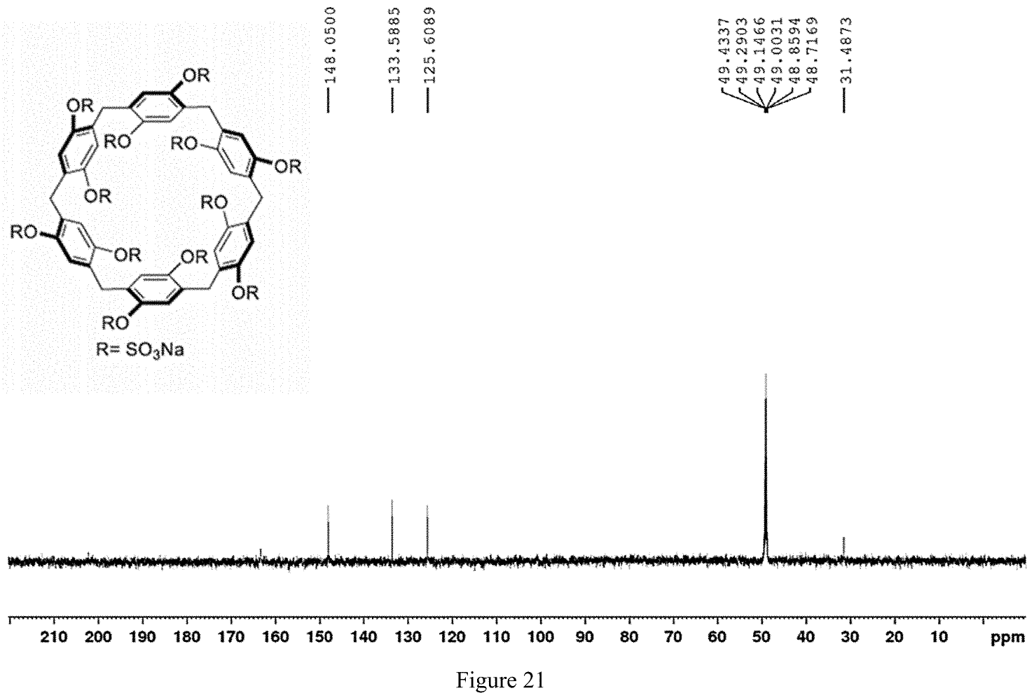

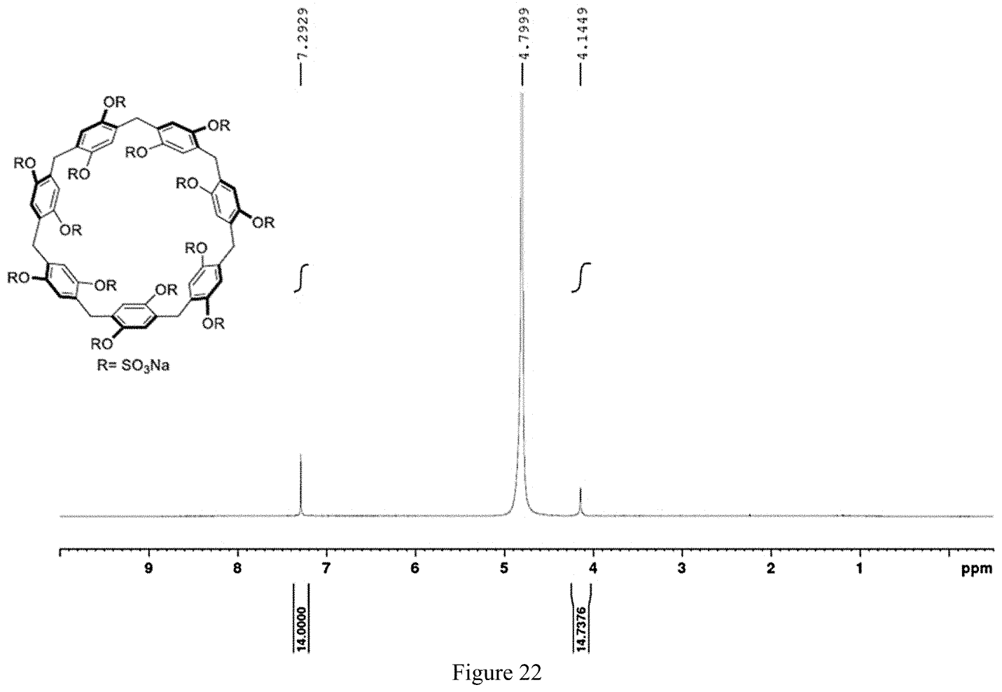

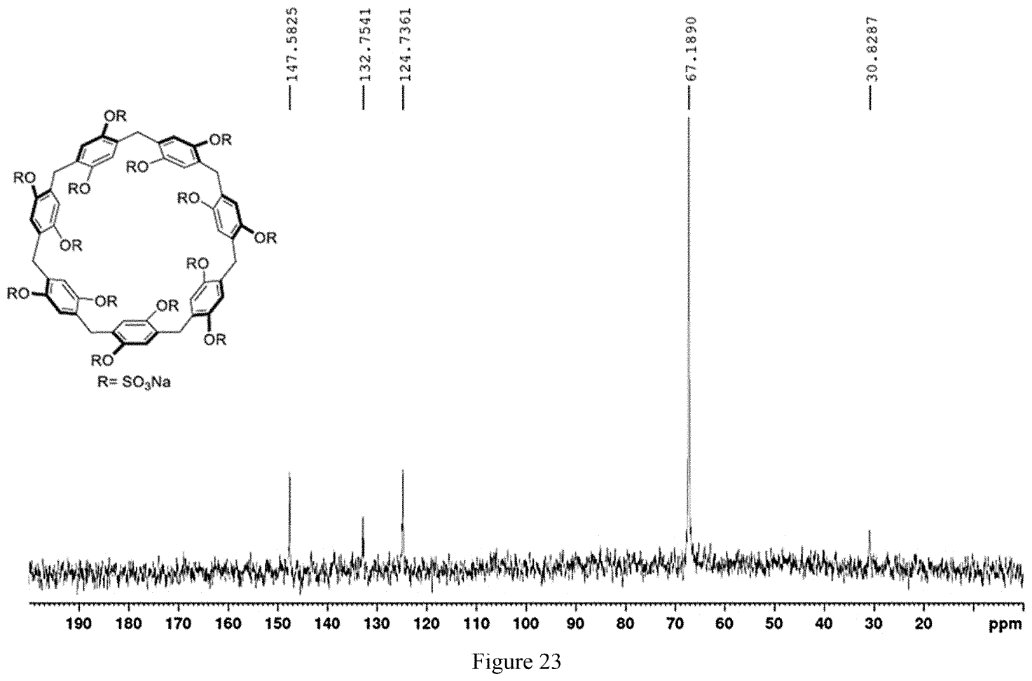

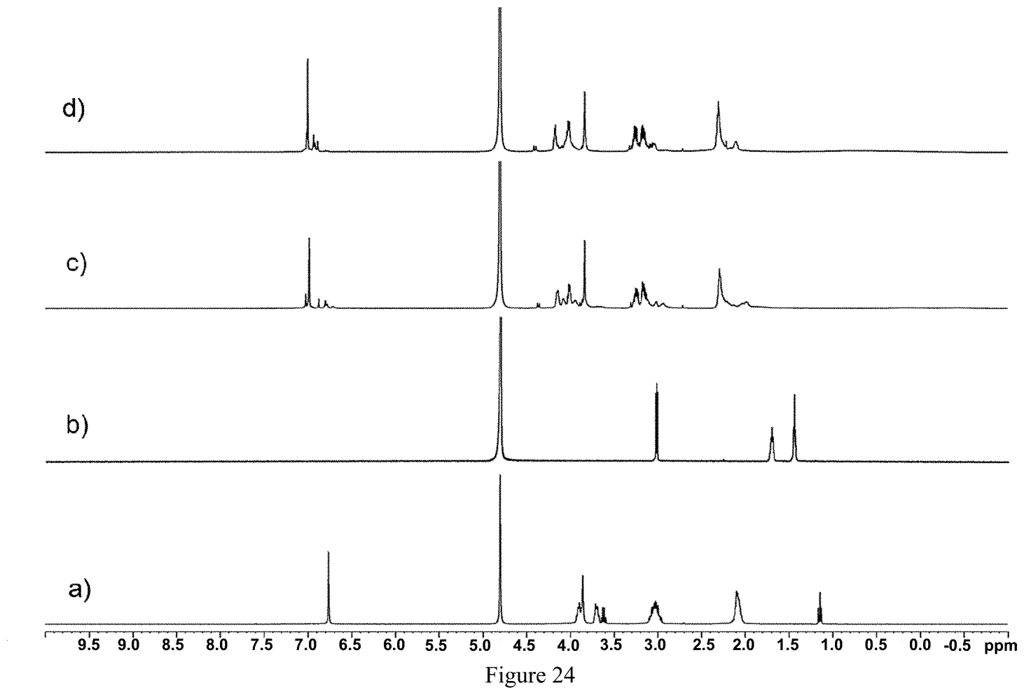

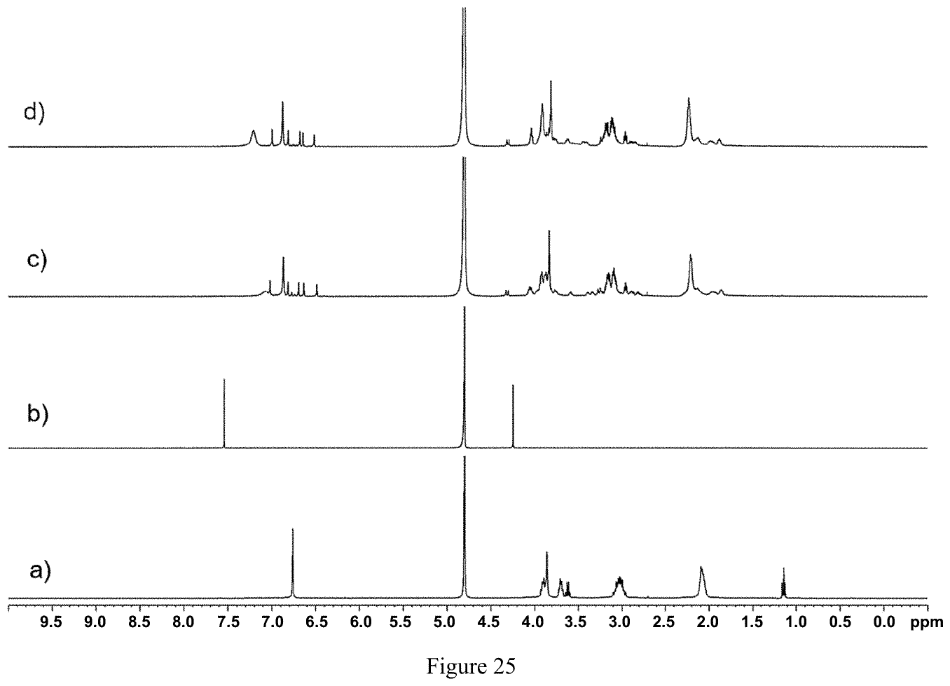

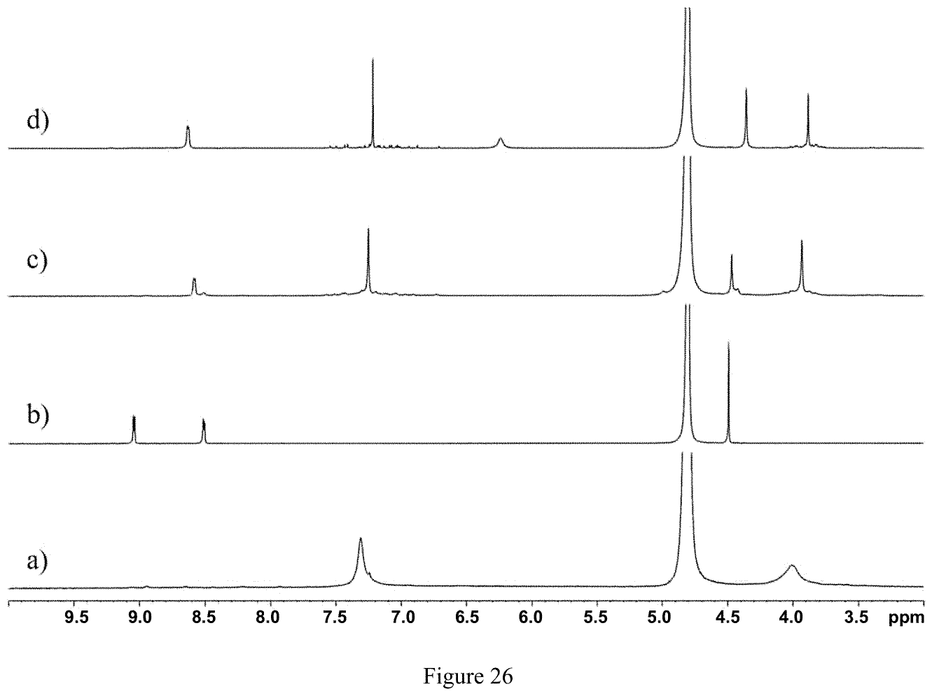

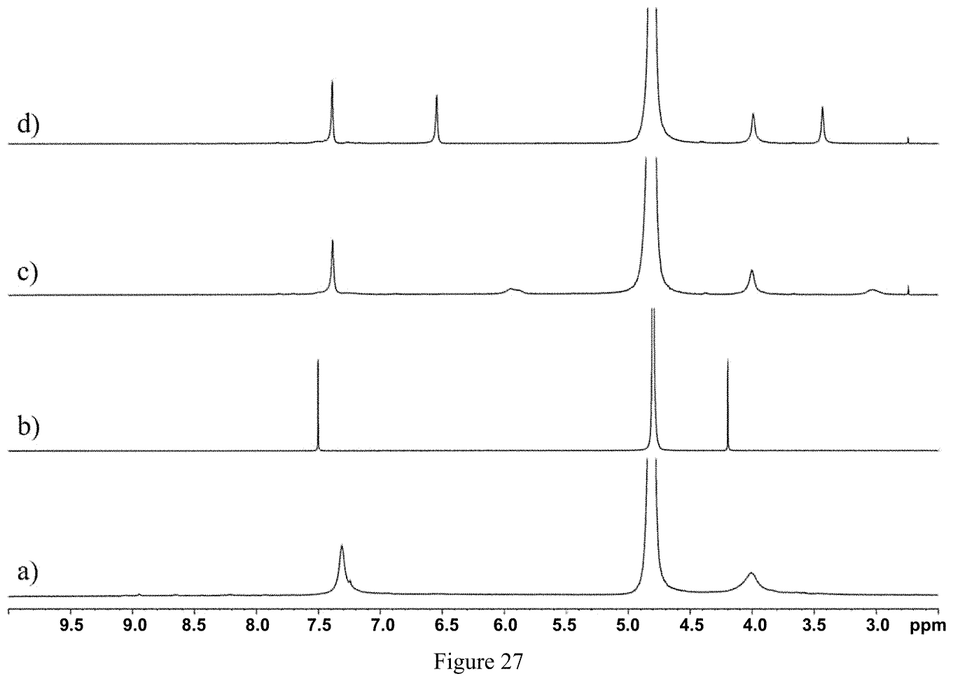

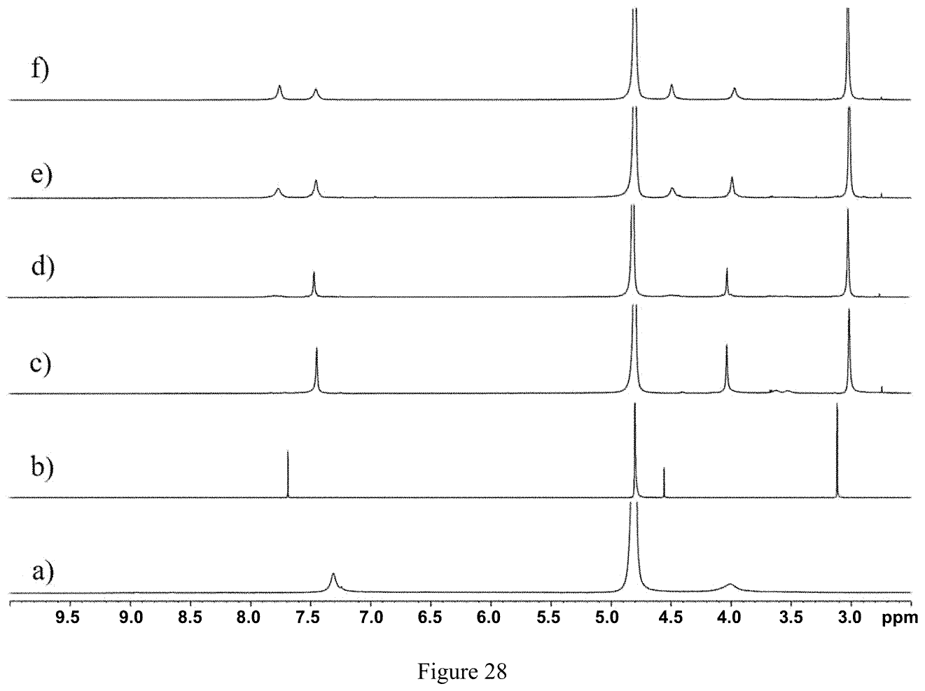

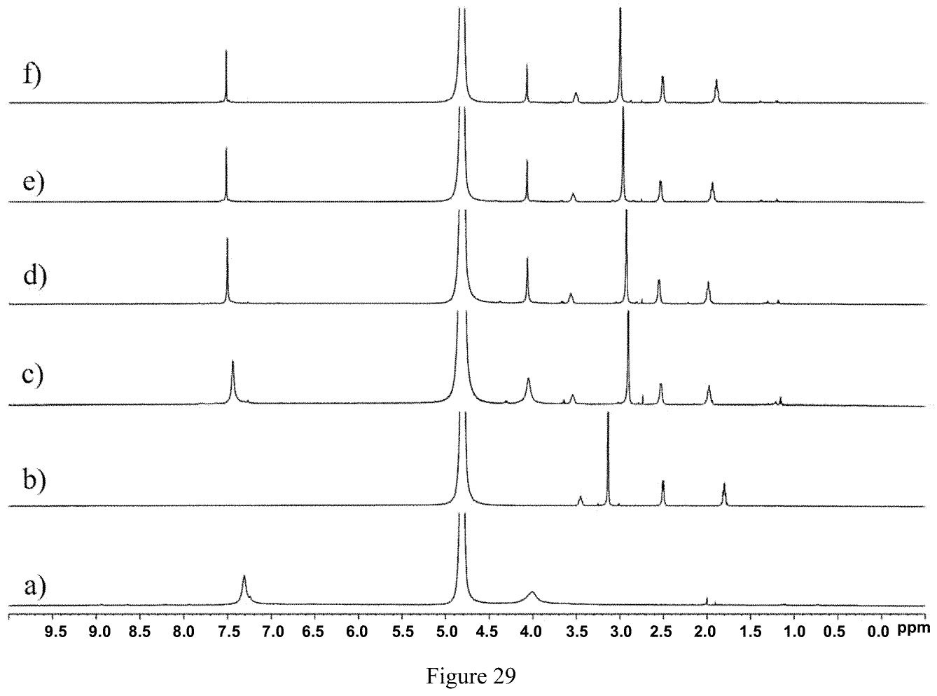

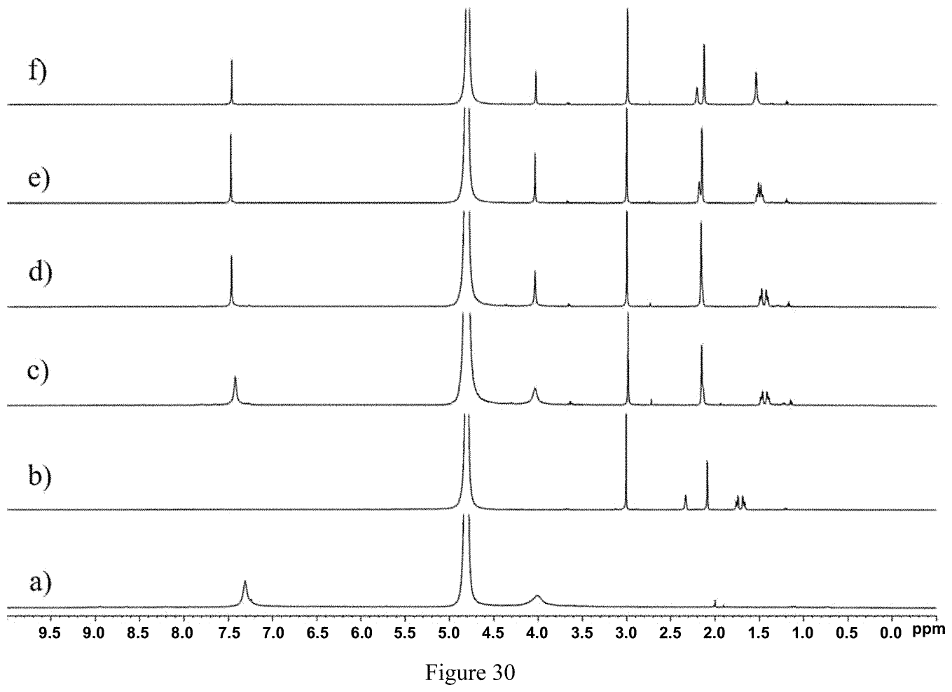

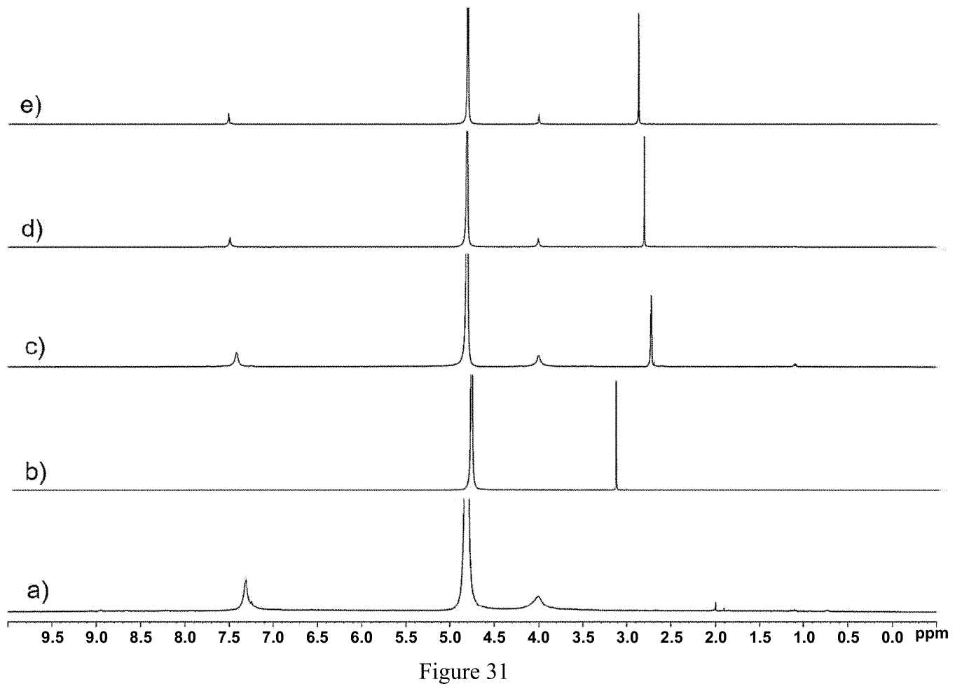

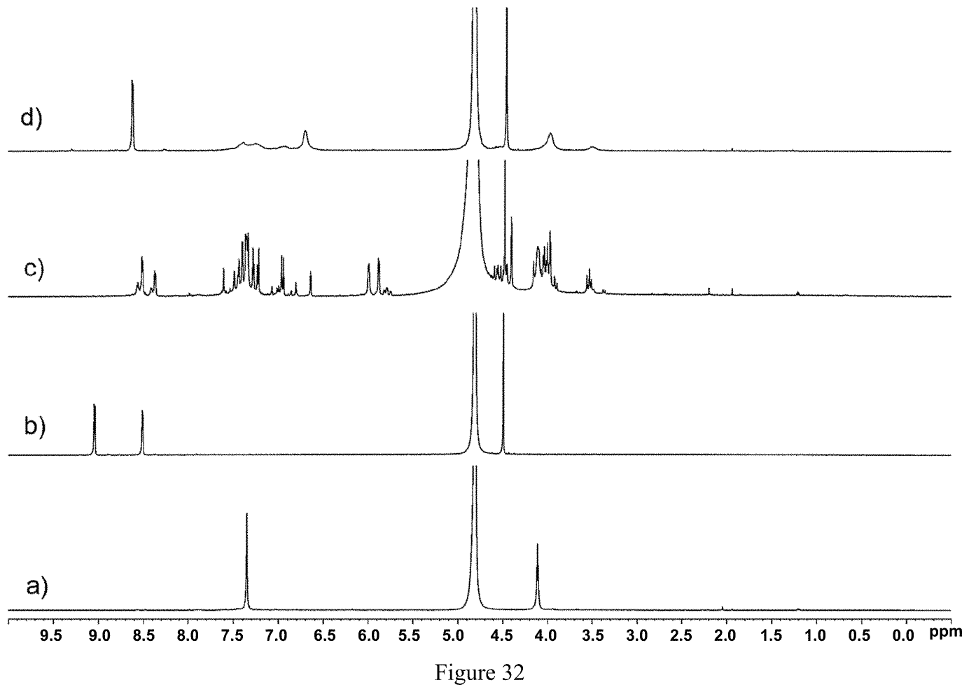

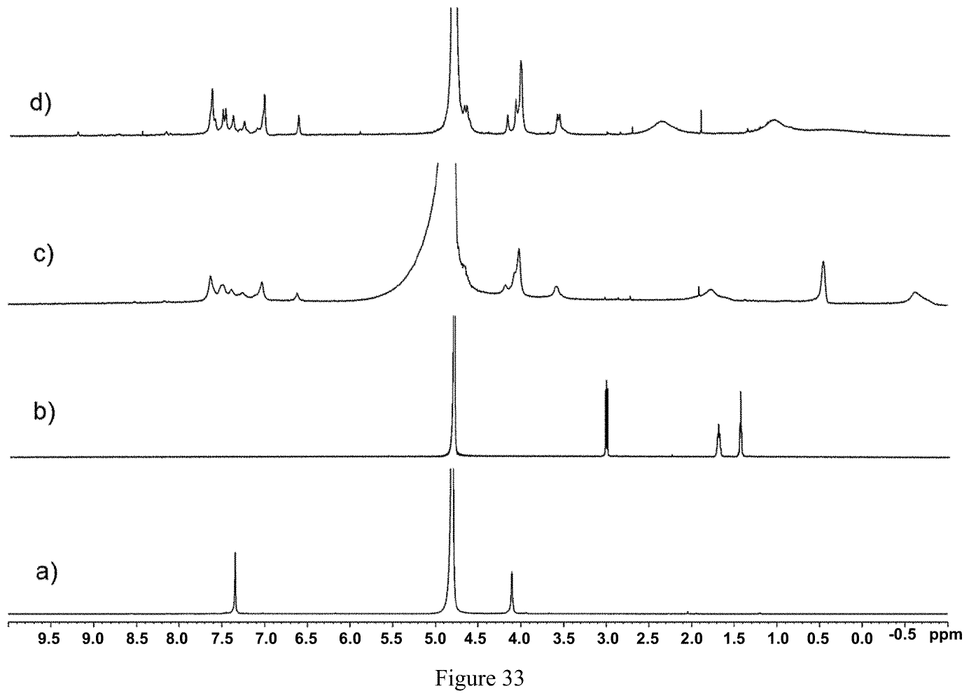

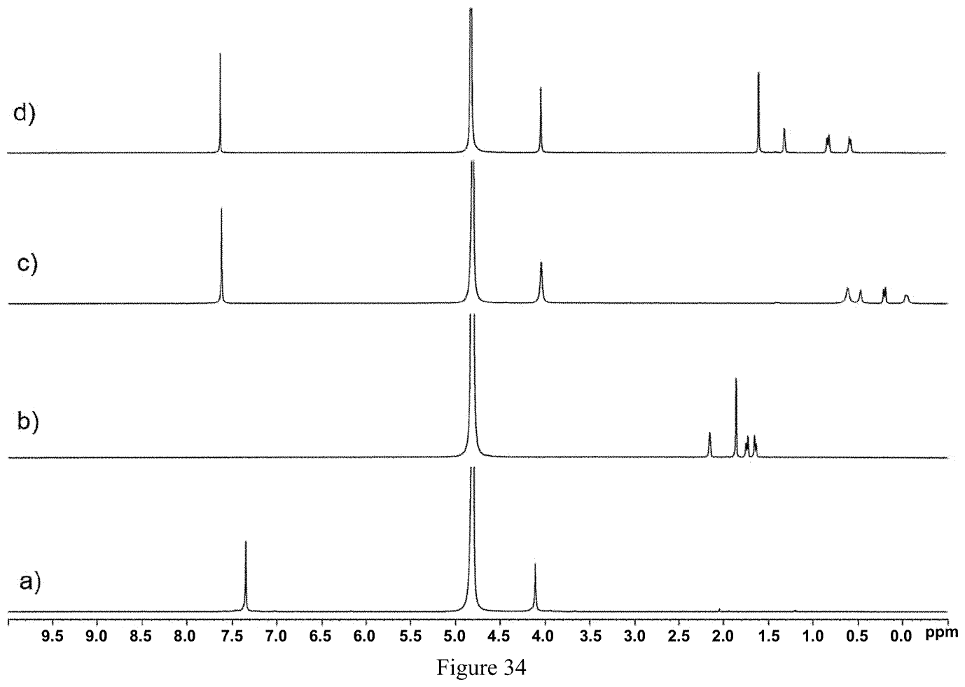

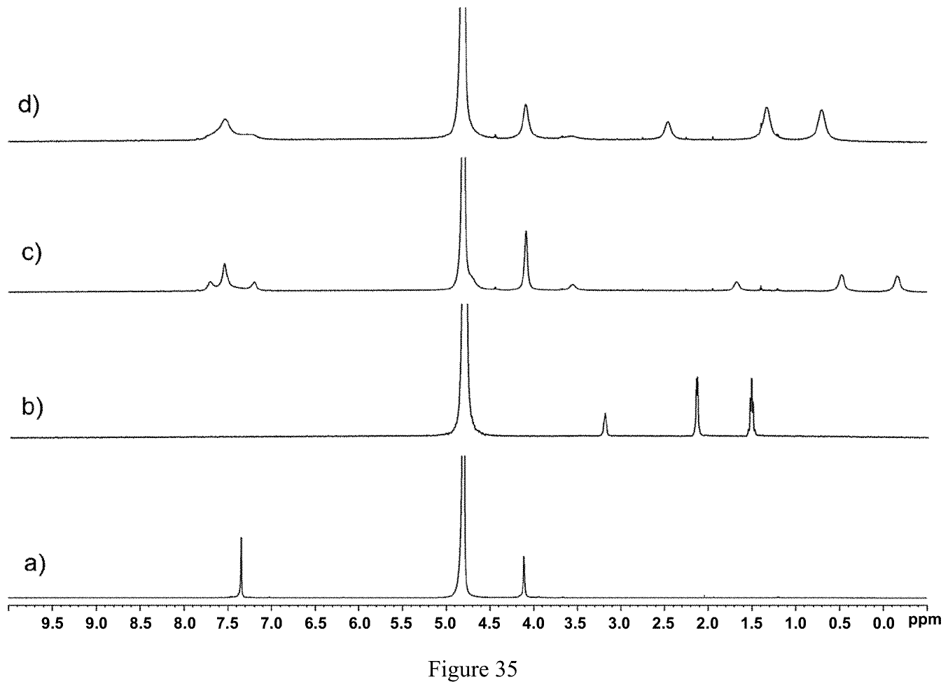

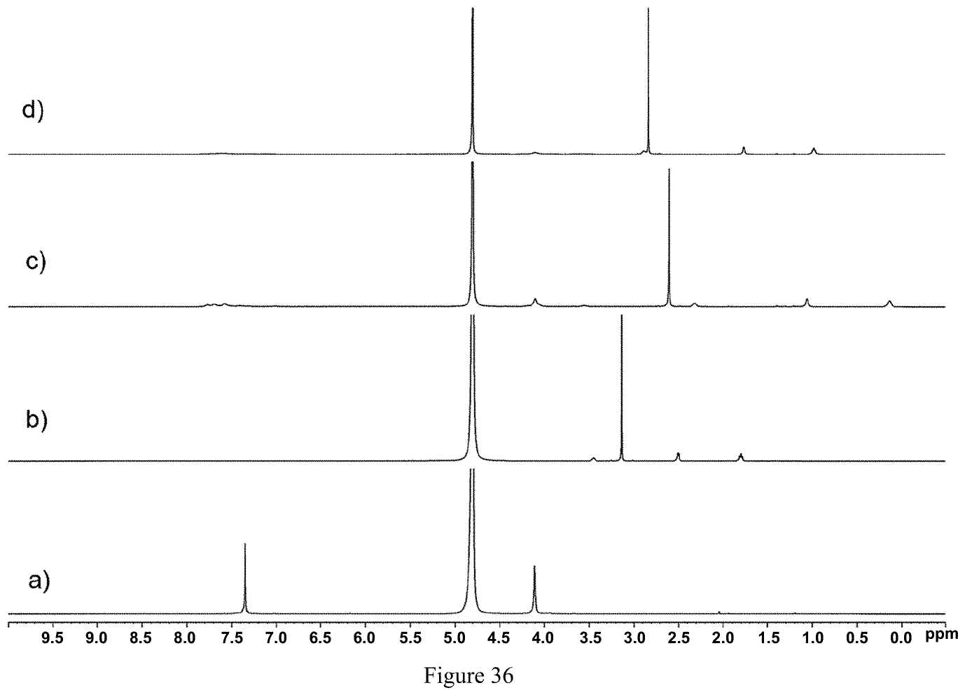

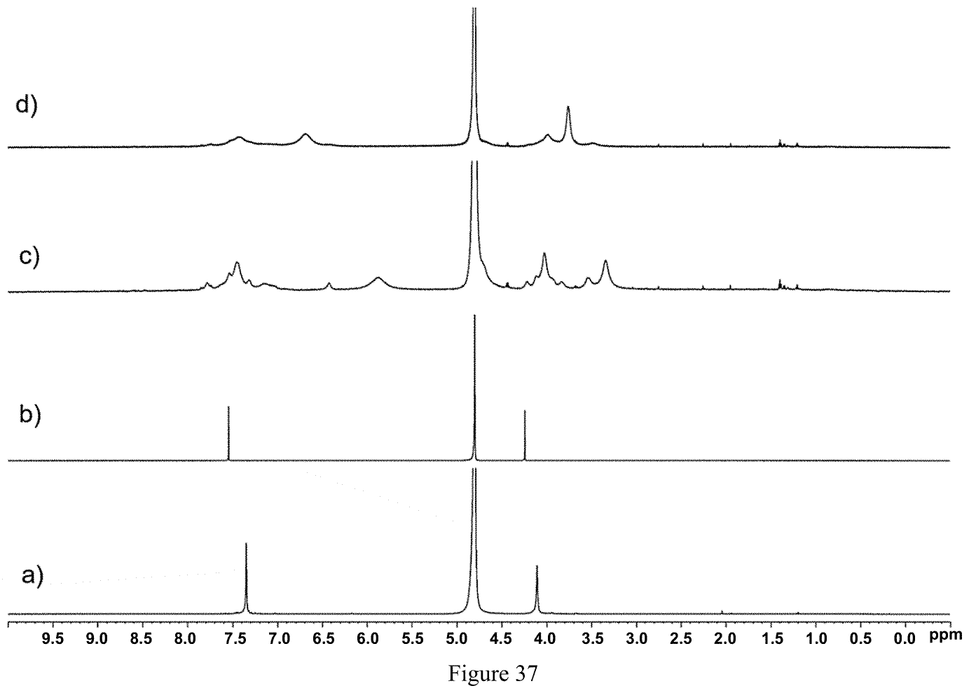

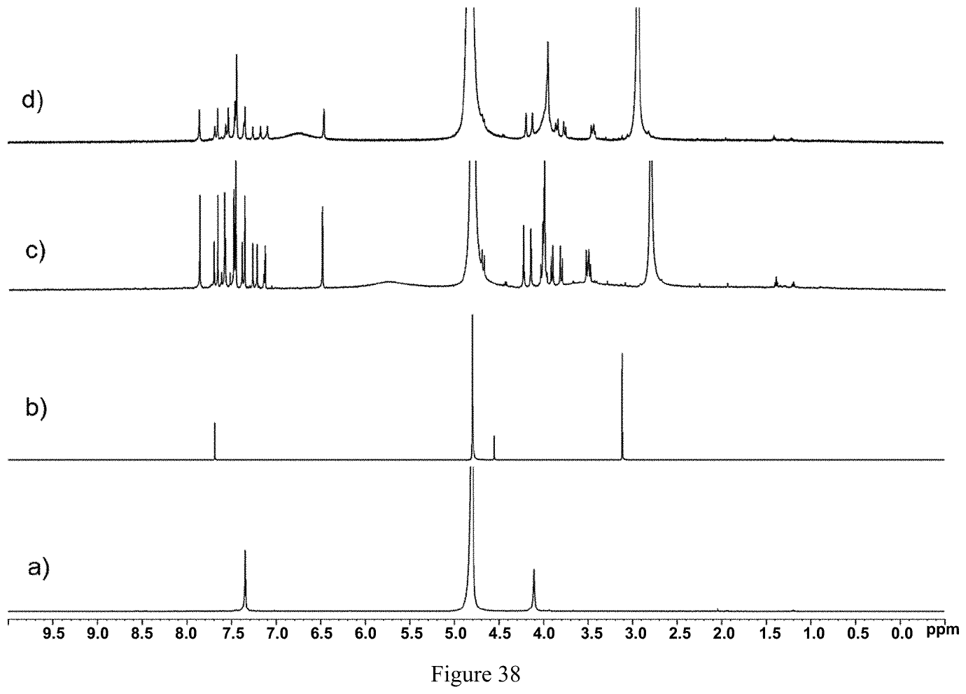

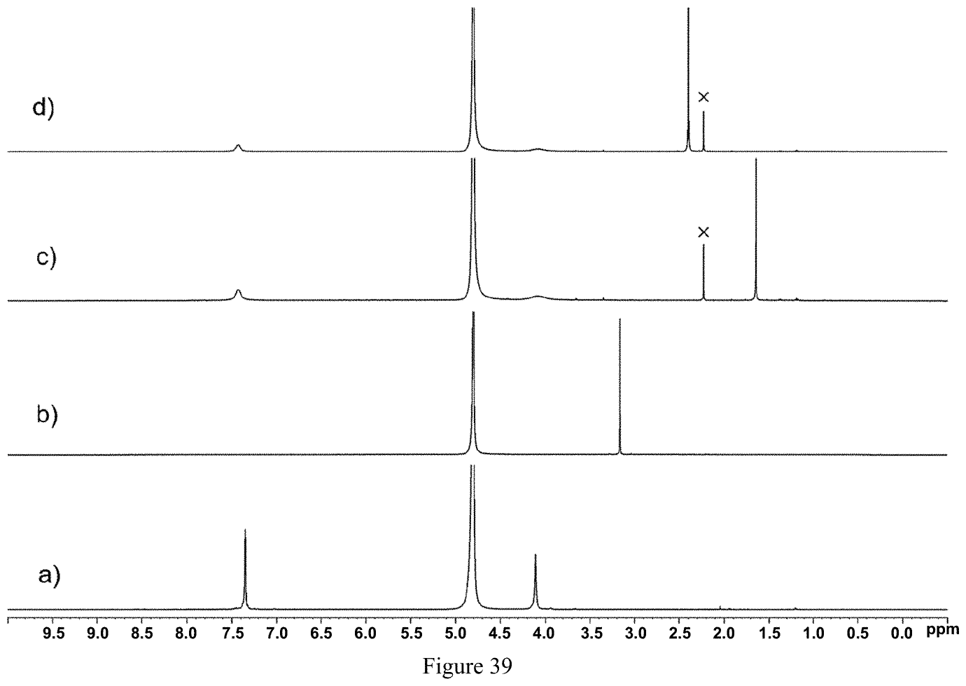

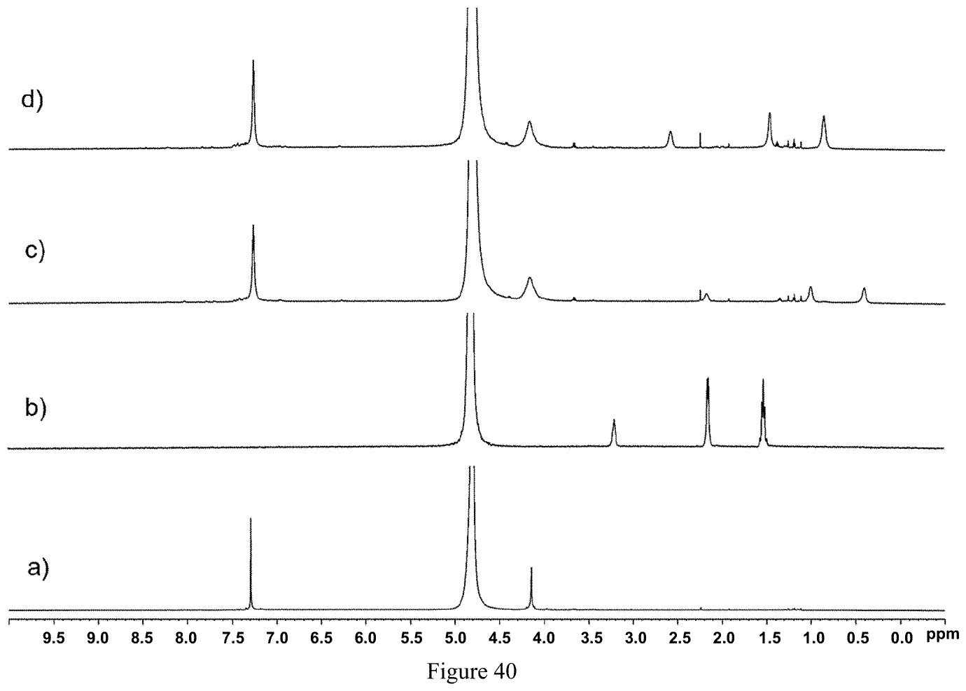

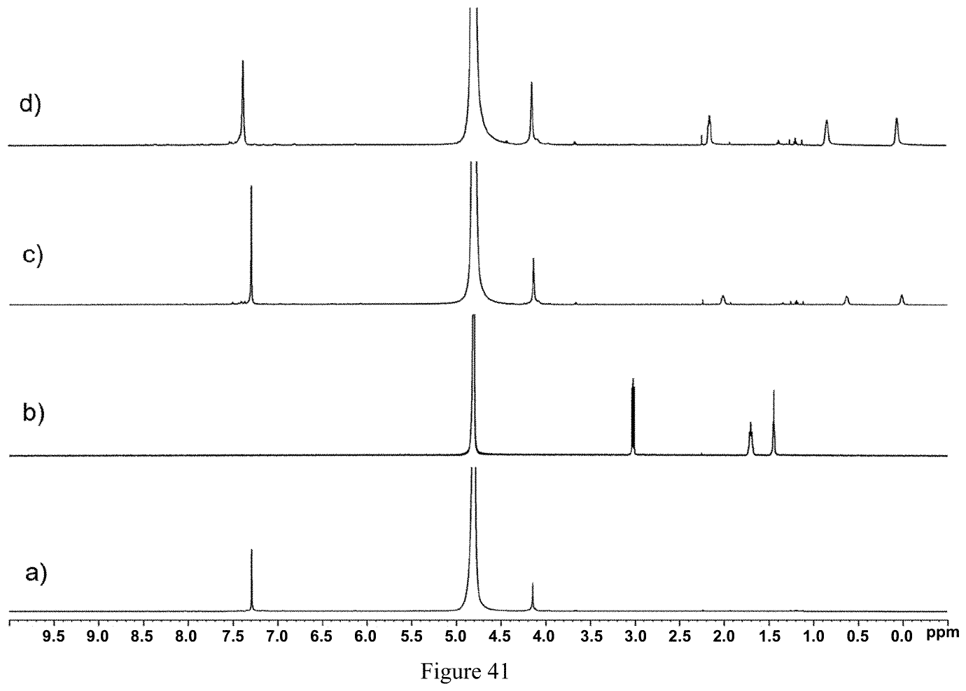

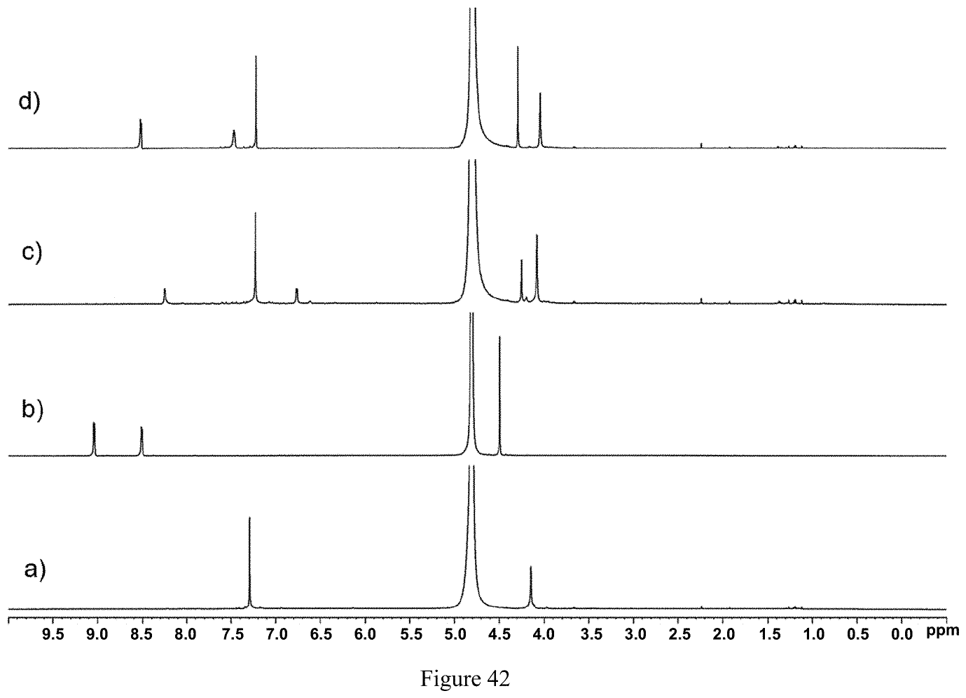

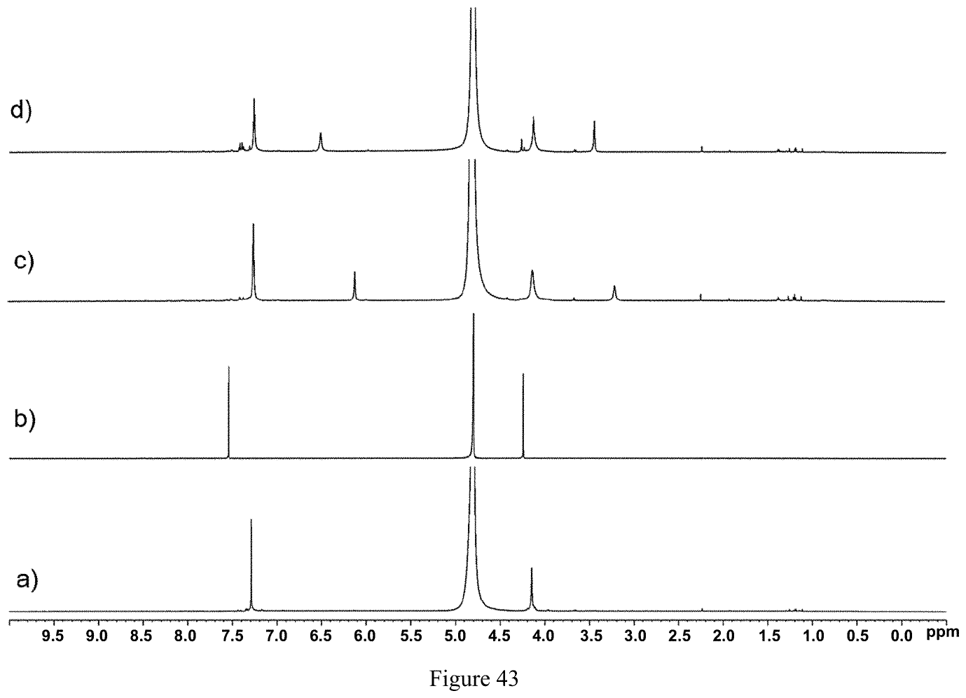

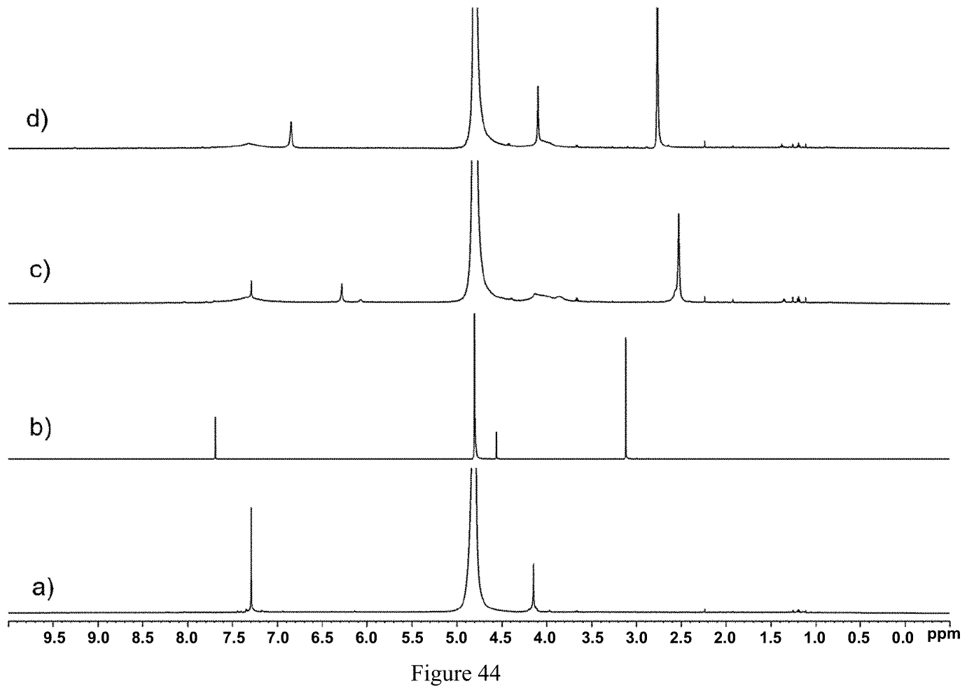

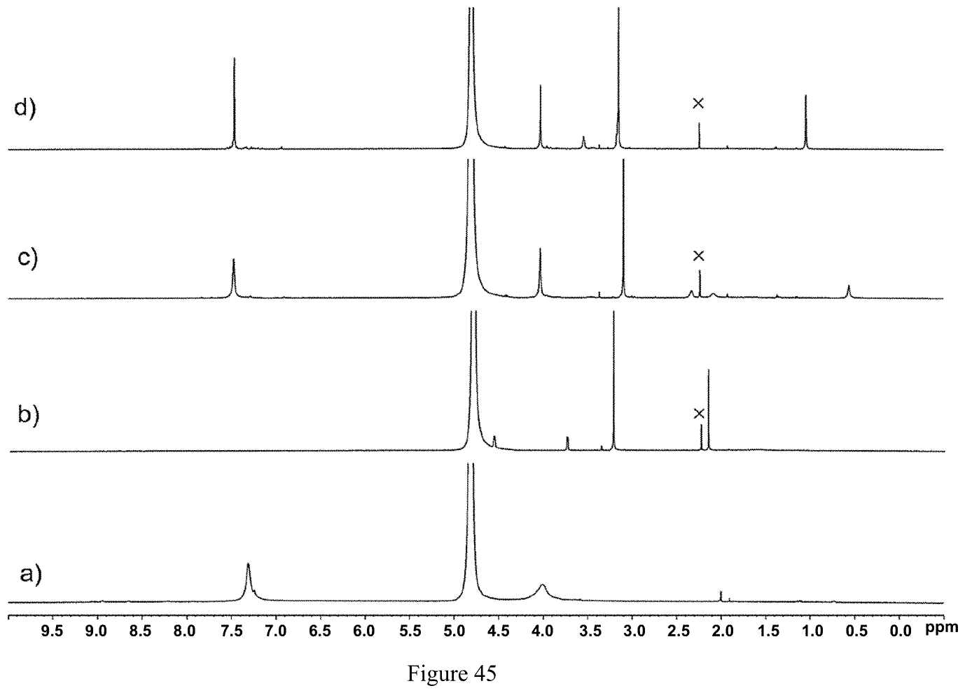

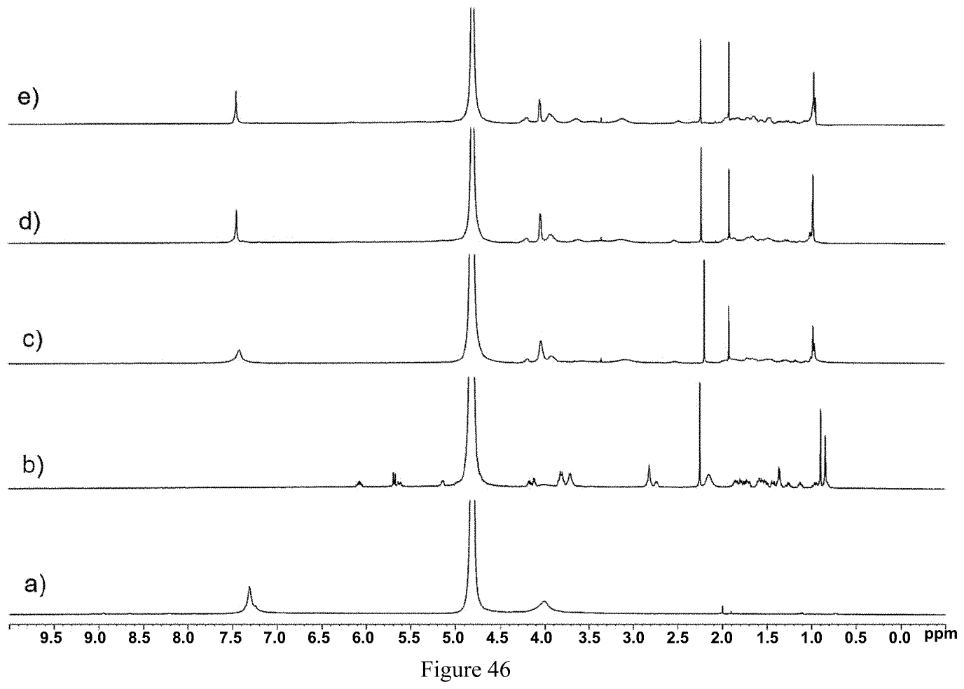

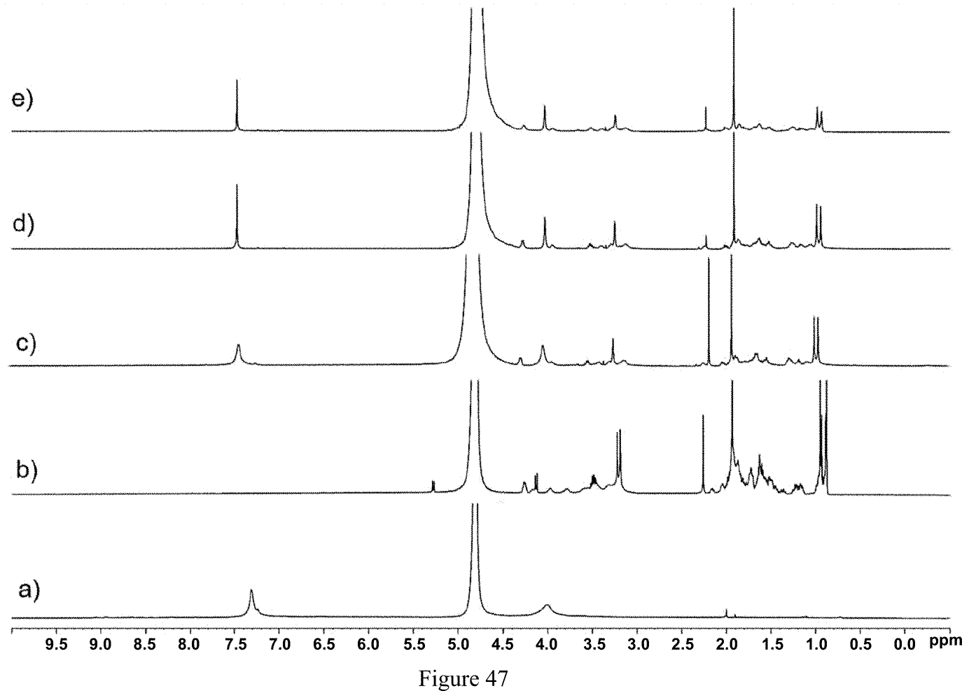

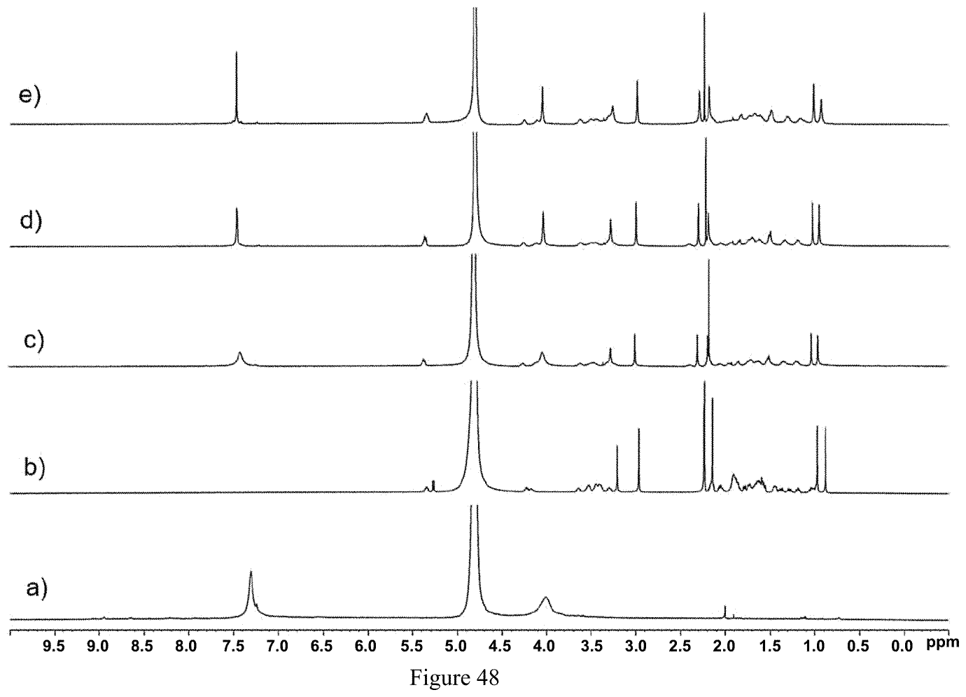

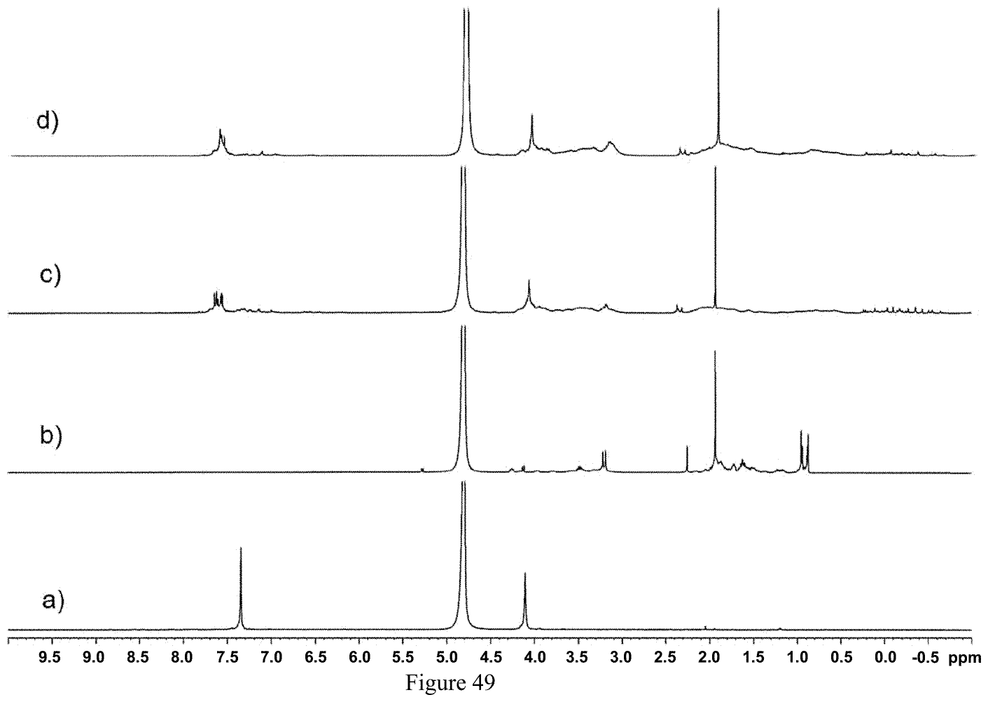

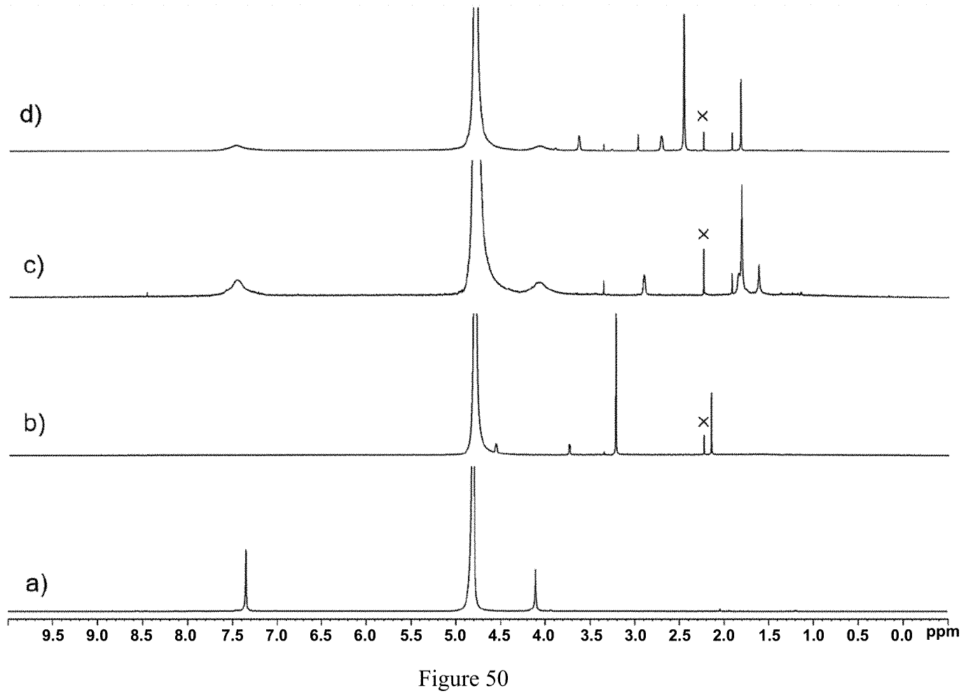

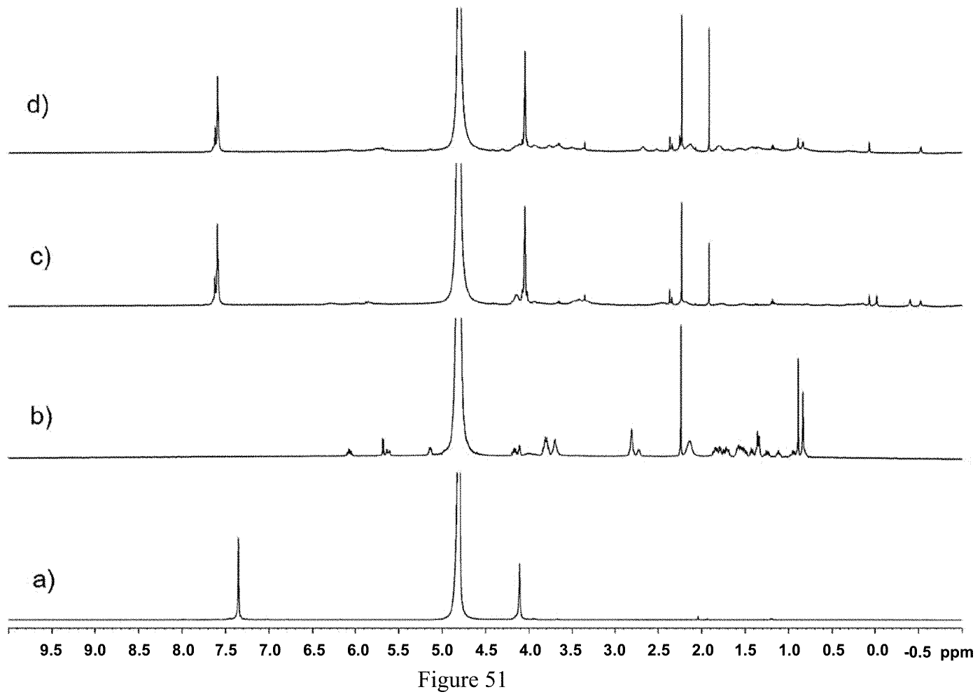

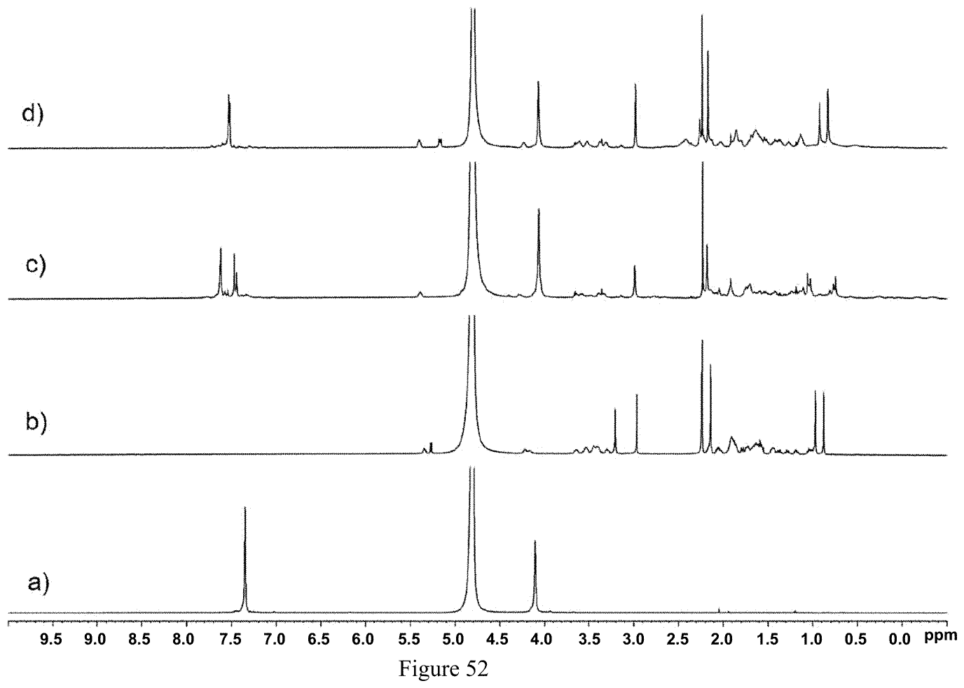

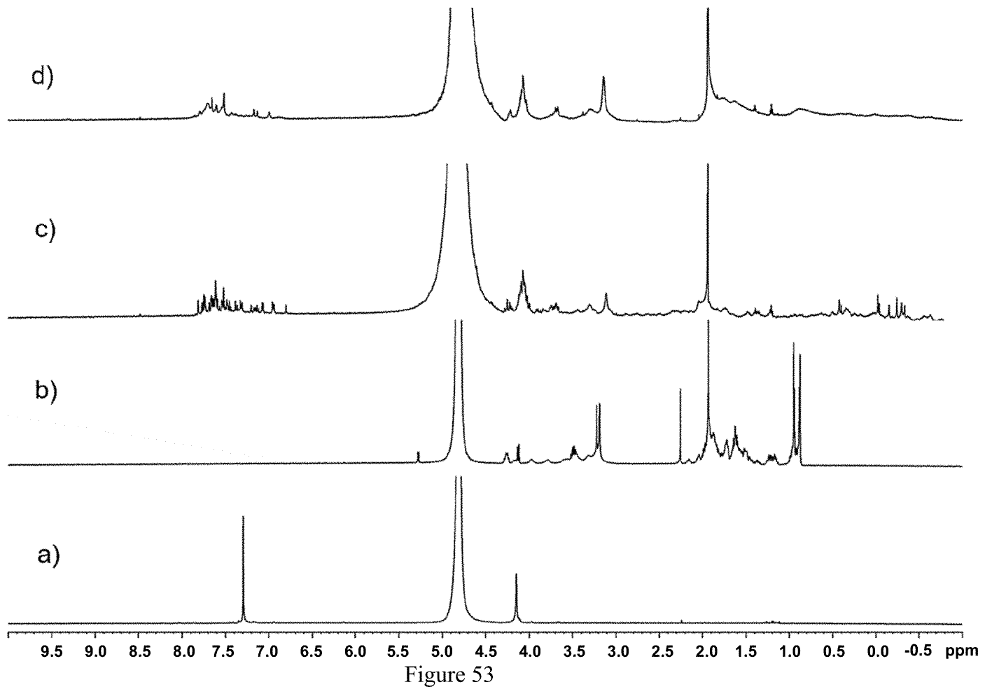

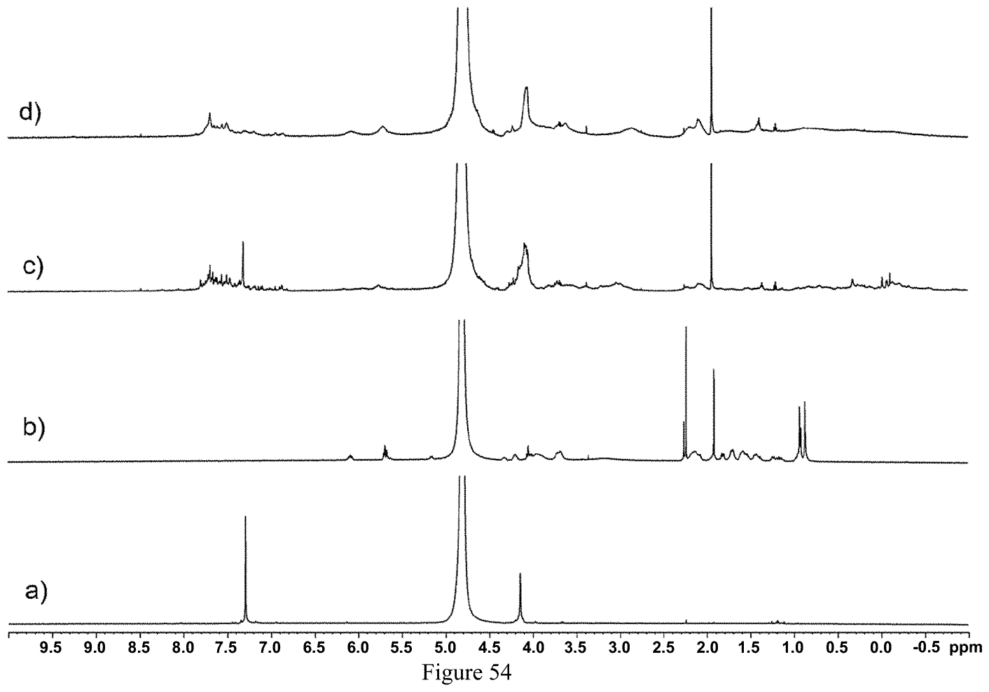

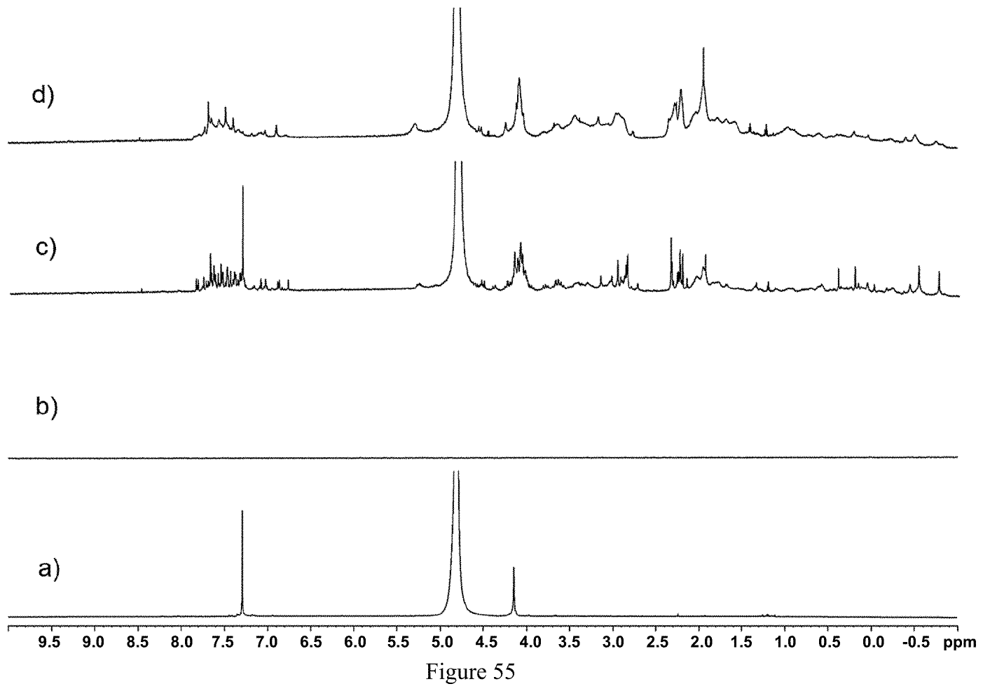

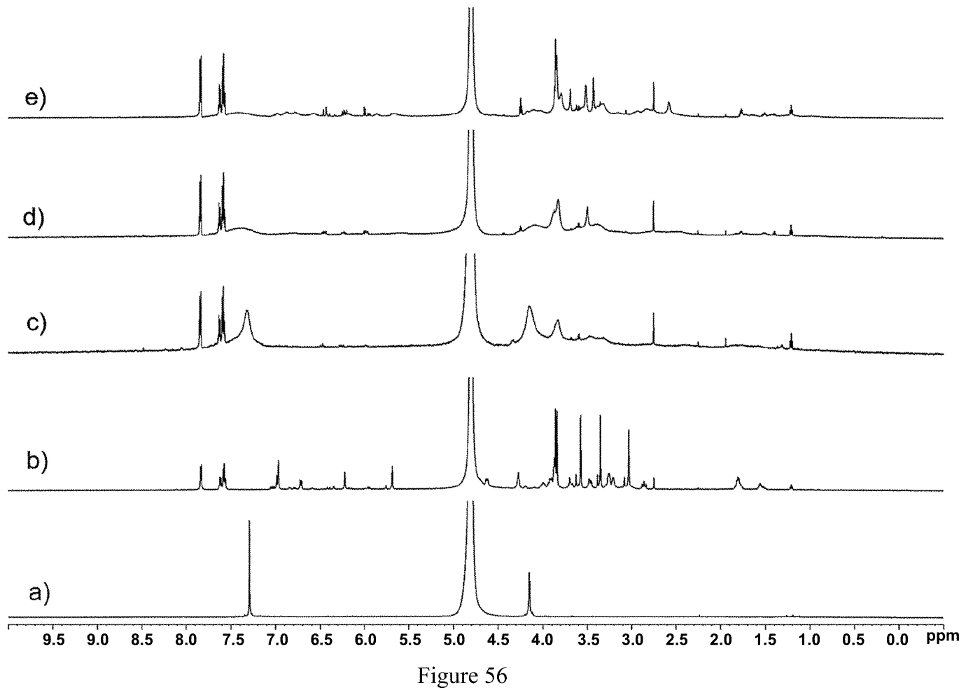

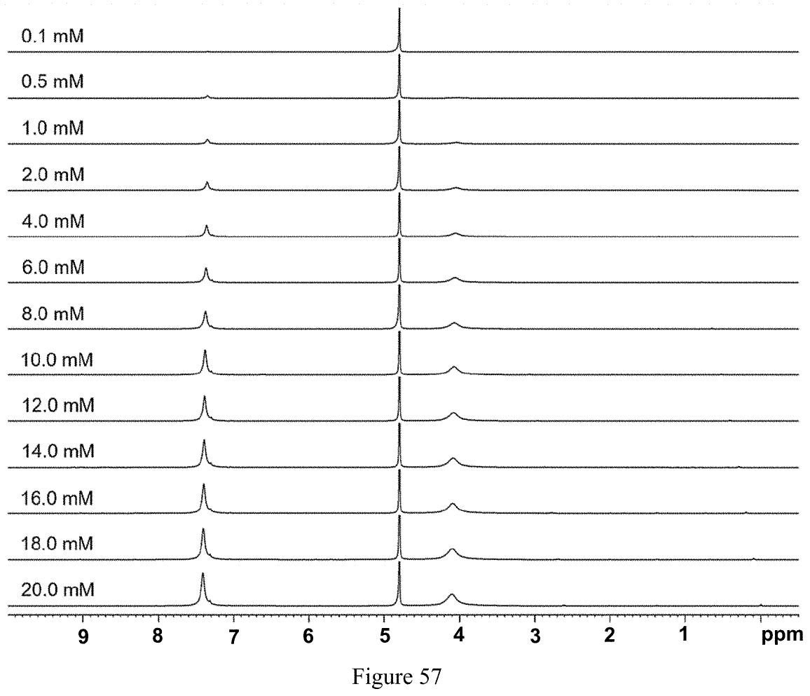

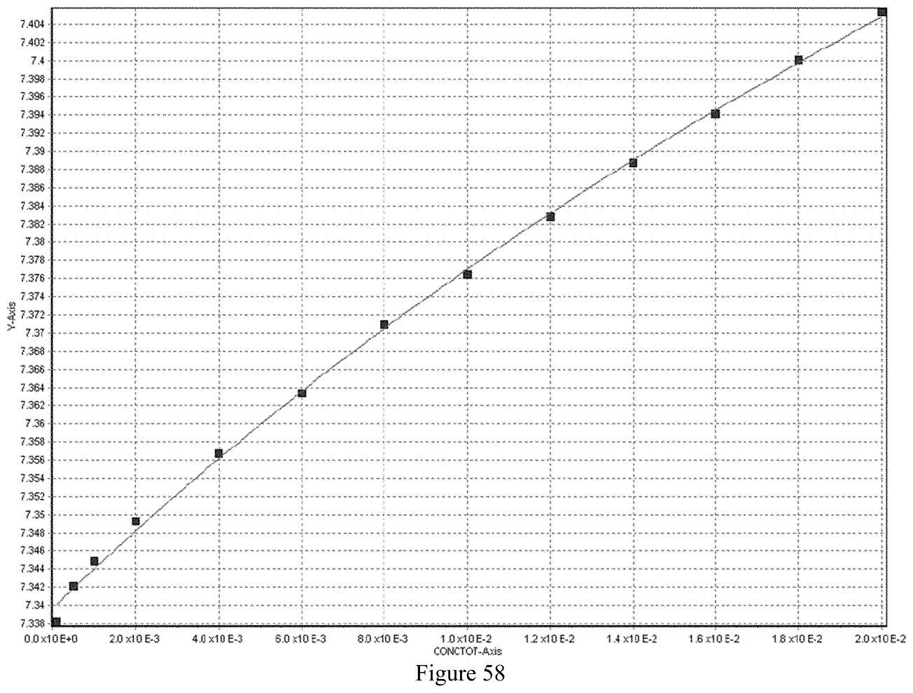

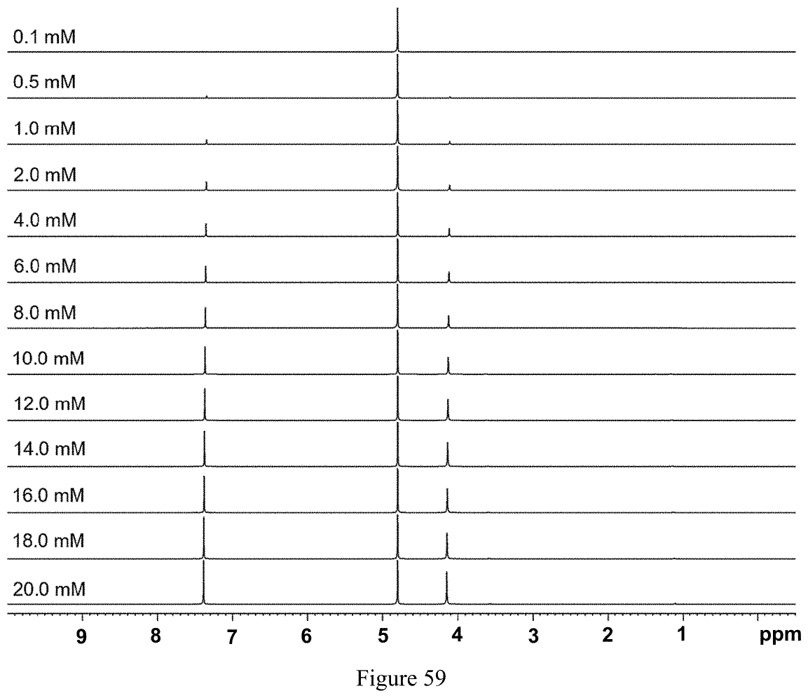

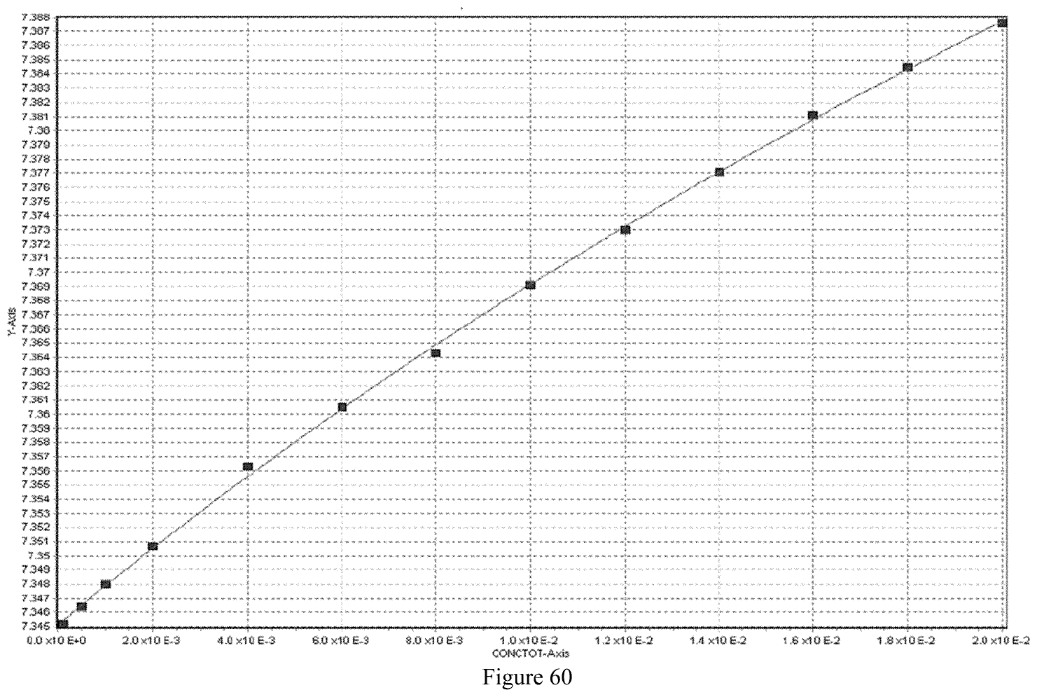

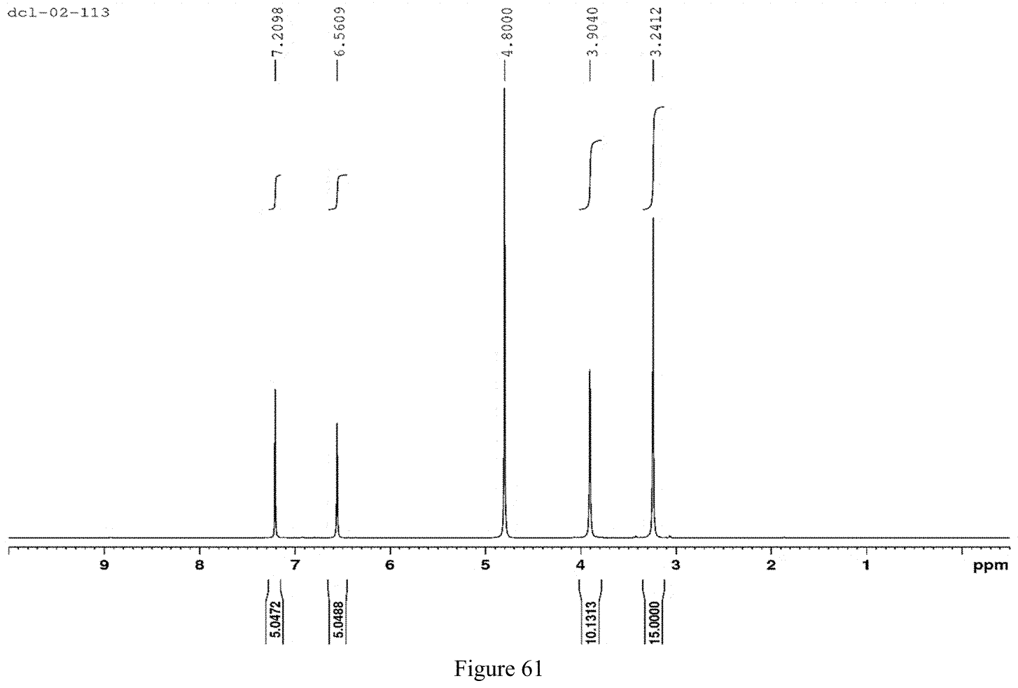

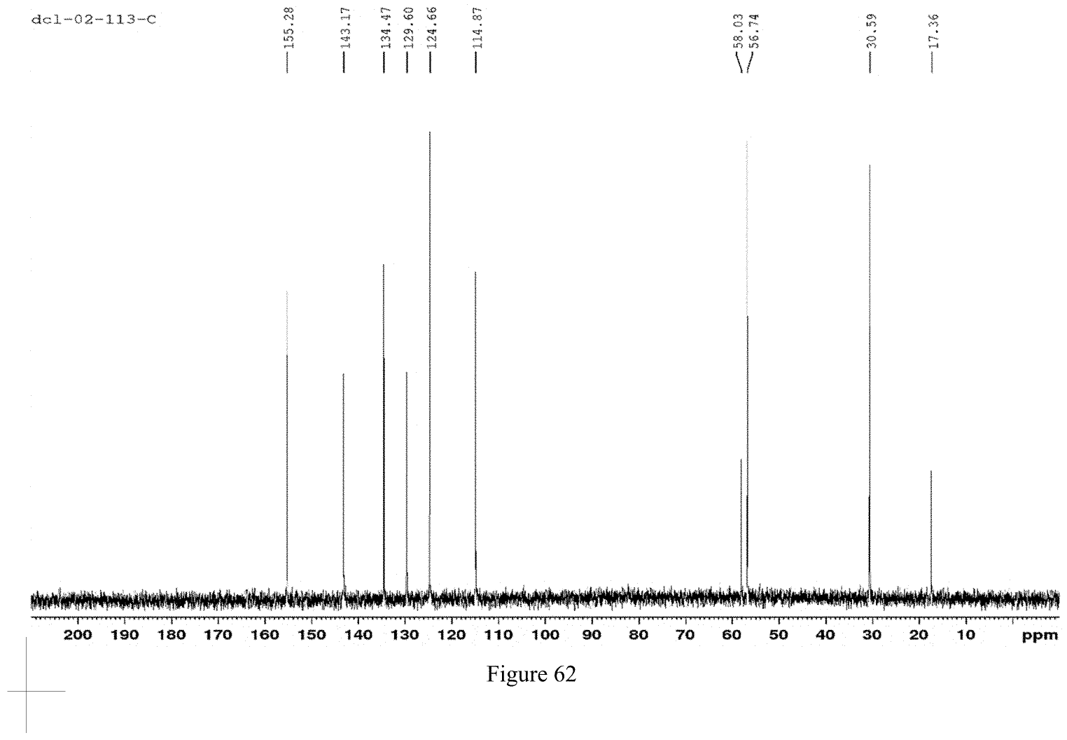

Given the demonstrated preference of P[5]AS and P[6]AS toward quaternary diammonium ions the guest panel extended to include the clinically important neuromuscular blocking agents roc, vec, pan, and cis as well as acetyl choline (ACh). Macrocyclic receptors (e.g., γ-cyclodextrin derivative Sugammadex marketed by Merck as Bridion™, acyclic CB[n]-type receptor M2, and WP[6]) have previously been used as in vivo sequestration agents for NMBAs. Accordingly, the binding affinities of P[5]AS-P[7]AS, WP[5], and WP[6] were measured toward the NMBAs (FIG. 7). Most strikingly, it was found that P[6]AS binds roc, vec, and pan 10⁴-10⁵-fold more tightly than WP[6] or Sugammadex while maintaining very good levels of discrimination against acetyl choline (10³-10⁴-fold), which is also present in the neuromuscular junction. In fact, P[6]AS displays>100-fold higher affinity toward roc, vec, and pan than previously reported host M2 (Kₐ: M2.roc=3.4×10⁹ M⁻¹; M2.vec=1.6×10⁹ M⁻¹; M2.pan=5.3×10⁸ M⁻¹), which has been demonstrated to successfully reverse the biological effects of roc, vec, and cis in vivo in rats. To further demonstrate the superior binding affinity of P[6]AS over M2 toward roc a head-to-head test monitored by ¹H NMR spectroscopy was performed. FIG. 15a-e shows the ¹H NMR recorded for uncomplexed P[6]AS, M2, and roc and the P[6]AS.roc and M2.roc complexes. For the M2.roc complex, there is splitting and downfield shifting of H_{a*} and H_{b*} into a total of 8 resonances for the enantiomerically pure complex. For both complexes, there substantial upfield shifts of the axial steroidal Me-groups (Hₚ and H_{q}) which allow monitoring of the composition of mixtures of these two competing host.guest complexes. FIG. 15f shows the ¹H NMR spectrum recorded when a solution of M2.roc (0.5 mM) was treated with 1 equivalent of P[6]AS. The loss of the resonances for M2.roc and the appearance of resonances for P[6]AS.roc further verify the superior affinity of P[6]AS in the context of neuromuscular blockers. Previously, only reversal the in vivo effects of cis was achieved in rats at higher doses of M2 (>40 mg kg⁻¹) due to the lower binding affinity of the M2.cis complex (Kₐ=4.8×10⁶ M⁻¹). Experimentally, it was found that P[7]AS and cis form a (P[7]AS)₂.cis complex where the benzylisoquinolinium endgroups are each complexed by a P[7]AS host. The ITC data for (P[7]AS)₂.cis could be fitted to a 1:1 binding model with Nₛᵢₜₑₛ=2 and Kₐ=1.52×10⁷ M⁻¹. Accordingly, P[7]AS has potential for translation into an in vivo reversal agent for cis.

In summary, this Example describes the synthesis of P[5]AS-P[7]AS, the x-ray crystal structures of P[5]ACS and P[6]AS, and their molecular recognition properties toward (di)ammonium ions in aqueous solution. P[n]AS packs 2n negative charges into a small volume near the portals of the receptors which augments the electrostatic contributions to binding free energy. It was found that P[5]AS and P[6]AS display significantly higher binding affinity than WP[5] and WP[6] toward (bis)quaternary (di)ammonium ions. Accordingly, the suggested family name is Pillar[n]MaxQ. The picomolar affinity of P[6]AS toward roc and vec greatly exceeds that of acyclic CB[n]-type receptor M2 and Sugammadex which is used in clinical practice under the trade name BRIDION™. The ultratight binding (e.g. picomolar Kₐ) displayed by P[5]AS and P[6]AS places them alongside CB[n] as some of the most potent synthetic receptors in water. The ultratight binding of P[5]AS and P[6]AS suggests that sulfated pillararenes and their functionalized derivatives may be used as non-covalent connectors for bioconjugation, in (bio)chemical separations, for theranostics, as well as for sequestration and remediation in chemical and biological systems.

Determination of Kₐ between various hosts and cationic guests using Isothermal Titration Calorimetry (ITC). All ITC experiments were conducted in the 200 μL working volume of the sample cell of the PEAQ ITC instrument. An injection syringe of 40 L capacity was used. In each case, the host and guest solutions were prepared in a 20 mM NaH₂PO₄ buffer (pH 7.4). The sample cell was filled to capacity (200 μL) with the host solution and the guest solution was titrated in (first injection=0.4 μL, subsequent 18 injections=2 μL). The binding data was fitted using the 1:1 binding model or the competitive binding models in MicroCal PEAQ-ITC analysis software.

### Example 3

This Example provides in vivo effects of P[6]AS on reversal of methamphetamine induced hyperlocomotion in a pertinent mouse model. This Example also provides results from an in vivo toxicology study of P[5]AS and P[6]AS.

Cell Cytotoxicity Data for P[5]AS and P[6]AS. To test the Cytotoxicity and Cell Viability of the above compounds we used two different assays: an MTS (CellTiter 96 AQueous Kit®) assay that measures cellular metabolism, and the AK (Toxilight®BioAssay Kit) assay that measures cell death through release of the cytosolic enzyme adenylate kinase into the supernatant. Both assays were performed with two different cell lines. HEK293 and Hep G2cells, are frequently used in drug toxicity studies. HEK293, a human kidney cell line, is used to evaluate the effect of the drug on the renal system and Hep G2, a human hepatocyte cell line, is used to assess the response of liver cells where drugs are metabolized. The MTS and AK assays for both cell lines were conducted after 24 h of incubation with the compounds at concentrations of 0.01 mM, 0.03 mM, 0.1 mM, 0.3 mM, and 1 mM. Eight technical replicates were designated for untreated cells and four technical replicates were designated for the cells treated with each compound and staurosporine (apoptosis inducer).

The collected absorbance and relative luminescence data were normalized to percent cell viability (MTS) and percent cell death (AK) using equations 1 and 2:

% cell viability=(Abs sample/Average Abs UT)×100 1)

% cell death=(RLU samples/Average RLU Distilled water)×100 2)

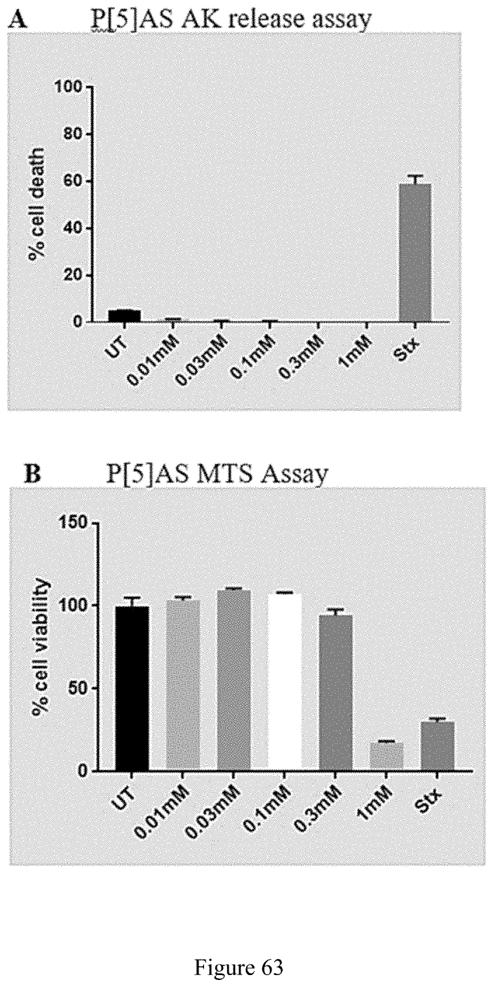

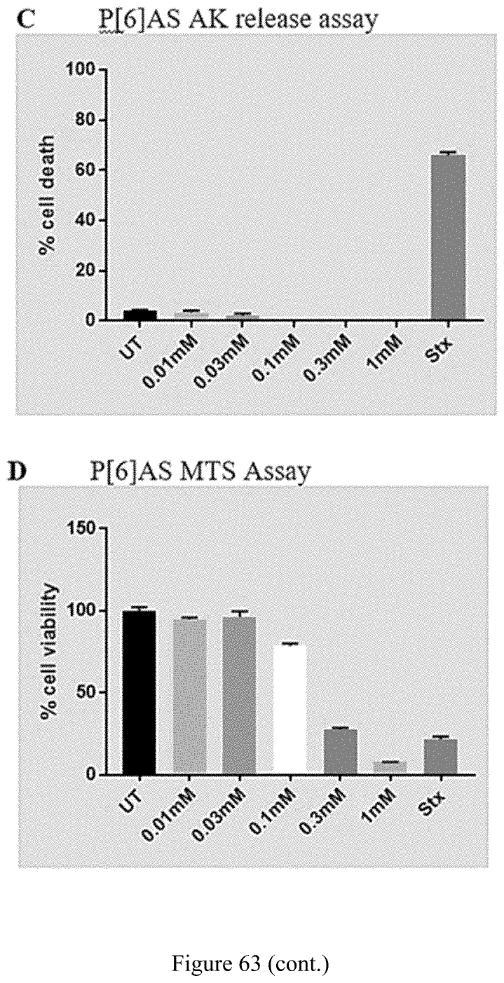

Toxicity studies using the MTS and AK assays for the liver cell line, HepG2 suggests that P[5]AS demonstrates low cytotoxicity up to a concentration of 1 mM and high cell tolerance up to a concentration of 0.3 mM (FIG. 63A,B). P[6]AS demonstrates low cytotoxicity up to a concentration of 1 mM with human HepG2 cells and high cell tolerance up to a concentration of 0.1 mM (FIG. 63C,D).

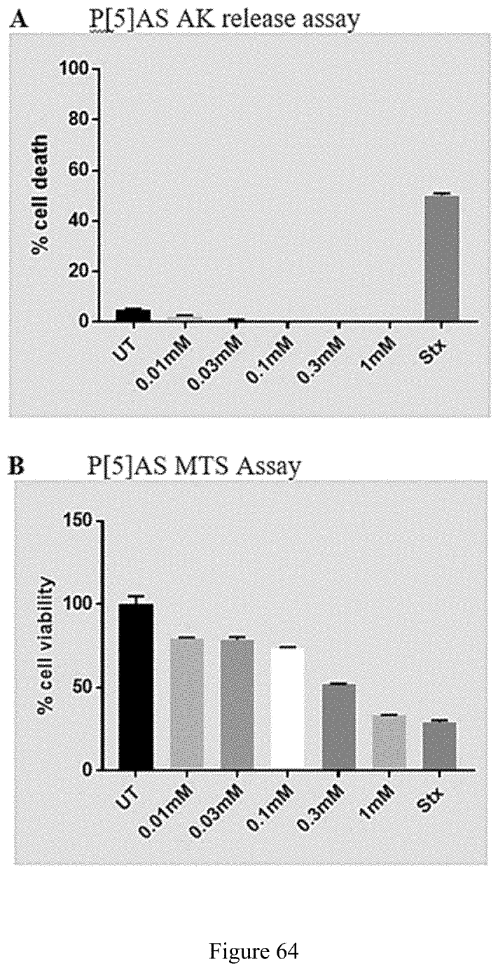

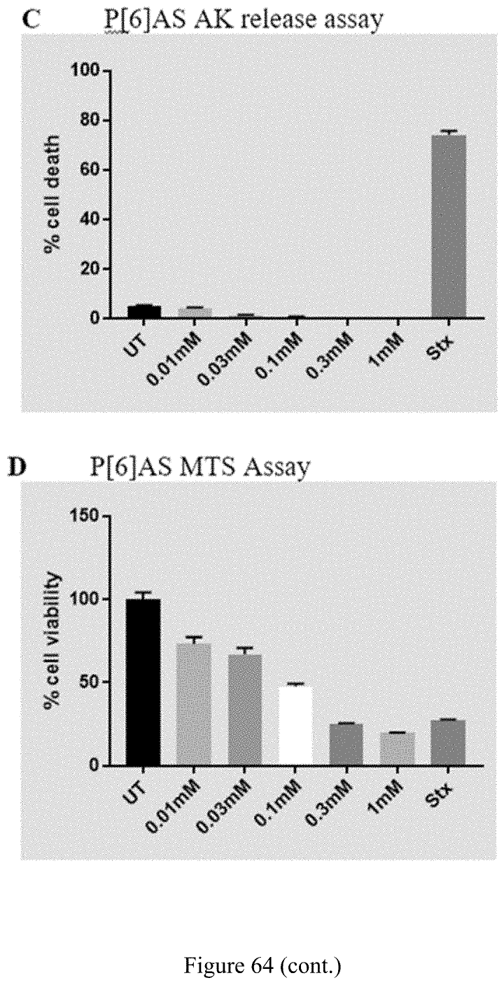

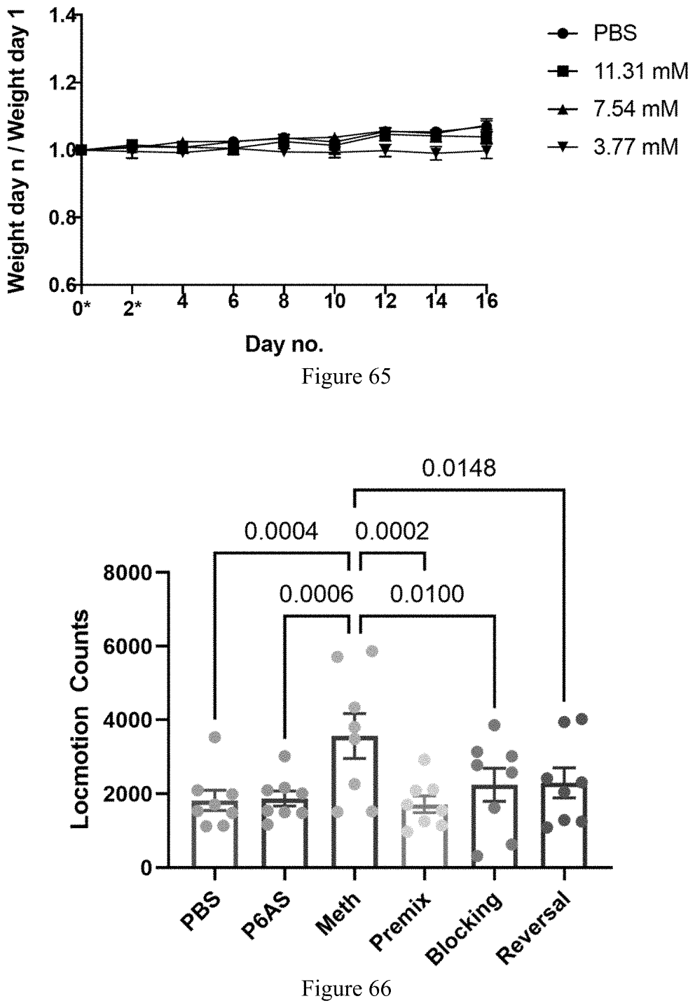

Similarly toxicity studies performed on human kidney (HEK293) cells suggest that P[5]AS demonstrates low cytotoxicity up to a concentration of 1 mM and high cell tolerance up to a concentration of 0.1 mM (FIG. 64A,B). P[6]AS demonstrates low cytotoxicity up to a concentration of 1 mM and high cell tolerance up to a concentration of 0.03 mM (FIG. 64C-D).

In Vivo Maximum Tolerated Dose Study (MTD). Animals studies were performed at the University of Maryland, Microbiology Building under the supervision of Dr. Volker Briken (IACUC #R-JAN-17-25). A total of 20 female Swiss Webster were used for this study. Three different concentrations of P[6]AS (11.31 mM, 7.54 mM, 3.77 mM) were used. A PBS control group was also included. Each concentration and control group contained 5 mice. The mice received the compound in 0.150 ml of PBS via tail vein injection, with 48 hours between injections. The weight and health status of the mice were monitored for 2 weeks following the last injection. Behavior summary: 11.31 mM dose group showed dose-dependent adverse effects in the form of freeze ups and some labored breathing. The 11.31 mM dose group returned to baseline behavior (that observed with the PBS control) ≈2-3 hours after injection. The lowest dose group 3.77 mM overall exhibited no adverse effects and behavior on par with the PBS control group.

MTD study performed for P[6]AS. Female Swiss Webster mice (n=5 per group) were dosed via tail vein on days 0 and 2 (denoted by *) with different concentrations of P[6]AS or phosphate buffered saline (PBS). The normalized average weight change per study group is indicated. Error bars represent SEM.

In Vivo Reversal of Methamphetamine Induced Hyperlocomotion by P[6]AS

Animals. Eight male Swiss Webster (CFW) mice were obtained from Charles River Laboratories that weighed ˜30 g upon arrival. Mice were individually housed in a temperature- and humidity-controlled room on a 12 h light/dark schedule with lights on at 6:00 am EST. For the duration of both experiments mice had ad libitum access to food and water. All behavioral testing occurred between 6:30 am and 2:00 pm EST, and all experimental procedures were approved by the University of Maryland Animal Care and Use Committee and conformed to the guidelines set forth by the National Research Council

Surgical Procedures. Mice were anesthetized with an intraperitoneal (IP) injection of ketamine (100 mg/kg)/xylazine (10 mg/kg) (n=8) and were implanted with jugular catheters with head-mounted ports. All surgical procedures were conducted using aseptic technique, with body temperature monitored and maintained throughout surgery. Catheters were placed in the right jugular vein with the port passed subcutaneously out towards the top of skull. Ports (5 MM Up Pedestal; P1 Technologies) were fixed to the skull with a combination of super glue (Loctite) and dental cement. Following surgery, mice received an immediate injection of Rimadyl (5 mg/kg) and 0.4 mL of warm sterile saline. Mice were treated post-operatively for two days with Rimadyl (5 mg/kg) and given a minimum of 5 days to recover before resuming training. Catheters were flushed daily with 0.1 mL sterile saline solution containing gentamycin (0.33 mg/mL) and 0.1 mL sterile saline solution containing heparin (20 IU/mL) in order to reduce clotting and maintain catheter patency. Catheter patency was assessed daily from the first day following surgery until the end of testing. Any mouse whose catheter exhibited significant flowback on a majority of days was excluded from analysis.

Behavioral Testing. Mice were trained on a standard autoshaping task described previously. All behavioral procedures were conducted in a Med Associates test chamber equipped with a food cup, a retractable lever, and 4 floor IR photobeams. Time stamps were generated from head entries into the food cup, downward deflections of the lever, or disruption of floor beams and recorded by the behavioral computer.

Mice were given one day of magazine training that consisted of the delivery of thirty 20 mg sucrose pellets (Bioserv) randomly delivered on a variable interval 30±15 schedule, in order to habituate mice to the box and pellet delivery. In order to minimize the impact of novelty-induced suppression of feeding, mice were given five to six 20 mg sucrose pellets each in their home cage for 2-3 days prior to the beginning of training.

Following magazine training, mice began Pavlovian training sessions, which consisted of the presentation of the lever (CS) for 8 s, which was immediately followed by the delivery of a sucrose pellet and the retraction of the lever. The CS was presented on a random interval of 90±30 s schedule. Each Pavlovian session consisted of 30 trials. Pavlovian training continued for 4 days prior to surgery. Following surgery and recovery, mice underwent Pavlovian training for an additional 8 days while being exposed to various treatments.

Experimental Design. P[6]AS's efficacy was assessed using a semi-counterbalanced design where all mice received each possible experimental treatment. The purpose of the experiments was to: (1) verify that binding of methamphetamine by P[6]AS would not be compromised in vivo, (2) verify that P[6]AS would not alter locomotor behavior, and (3) to demonstrate that P[6]AS can sequester methamphetamine in vivo. On the first day, regardless of experiment, mice underwent a refresher session free of treatment. On the following six sessions mice were treated with one of six possible treatments: 0.01M PBS (0.2 mL infused), P[6]AS only (4 mM; 0.178 mL infused), methamphetamine only (0.5 mg/kg; 0.022 mL infused), a premixed solution of P[6]AS and methamphetamine (Premix; ˜7:1 P[6]AS:Meth; 0.178 mL P[6]AS+0.022 mL Meth infused), P[6]AS followed by methamphetamine administered 30 s later (0.178 mL P[6]AS, 0.022 mL Meth infused), and methamphetamine followed by P[6]AS administered 30 s later (0.022 mL Meth, 0.178 mL P[6]AS infused). Mice only received only one infusion per day. The dose of methamphetamine was chosen based on previously published values that observed reliable hyperlocomotion in mice. It was sought to choose smallest dosage that reliably induced hyperlocomotion.

Following completion of the first six sessions, mice completed another two days of behavioral testing. On day 7, half of the mice (n=4) received P[6]AS followed by methamphetamine administered 5 minutes later (0.178 mL P[6]AS, 0.022 mL Meth infused), followed by infusion of methamphetamine followed by P[6]AS administered 5 minutes later (0.022 mL Meth, 0.178 mL P[6]AS infused) administered on the eighth day of testing. The other half of the mice (n=4) received the same exact treatment but in reverse order across days 7 and 8.

For each experiment, total locomotion counts (i.e., the total number of beam breaks) were obtained for each mouse across the entirety of each training session. For each experiment, locomotion counts were then analyzed across treatments using one-way repeated measures ANOVAs with tukey-corrected pairwise post-hoc t-tests in Graphpad Prism (Version 9.0.0).

In vivo reversal of methamphetamine-induced hyperlocomotion effects observed after 5 minute delay between treatment with methamphetamine and P[6]AS administration. On day 7 and 8 mice (n=8) received methamphetamine followed by an infusion of 0.01M PBS administered 5 minutes later (REV-C; 0.022 mL Meth, 0.2 mL PBS infused) or methamphetamine followed by P[6]AS administered 5 minutes later (REV-5; 0.022 mL Meth, 0.178 mL P[6]AS infused) in counterbalanced manner. Administration of P[6]AS 5 minutes after exposure to methamphetamine reduced hyperlocomotion (paired t-test, t(7)=2.757, p=0.0282). Bars represent average locomotion counts. Error bars represent the standard error of the mean (SEM). Dots represent counts for each mouse (n=8).

It will be recognized from the foregoing that this Example provides an analysis of the efficacy of P[6]AS in the sequestration of methamphetamine in vivo. Eight male Swiss Webster (CFW) mice were trained on an Pavlovian autoshaping task described previously and locomotion values were obtained and analyzed accordingly. To establish methamphetamine induced hyperlocomotion and examine the efficacy of P[6]AS mice were first treated single infusions of PBS (0.01M), P[6]AS only, methamphetamine only, a premixed solution of P[6]AS and methamphetamine, P[6]AS followed by methamphetamine administration 30s later, or methamphetamine followed by P[6]AS administered 30s later in counterbalanced manner. FIG. 66 depicts the results of this experiment by plotting locomotion counts as a function of treatment. Mixed effects analysis revealed a significant main effect of treatment (F(5,35)=7.116, p=0.0001) with Tukey-corrected post-hoc comparison showing a significant increase in locomotion counts for treatment with methamphetamine against all other treatments (p's<0.05). Critically, there was no difference in locomotion for the comparison between reversal (i.e., meth first, followed by P[6]AS 30s later) suggesting that P[6]AS on its own has no negative effect on locomotor behavior, and that sequential administration of P[6]AS reduces methamphetamine-induced hyperlocomotion to control levels.

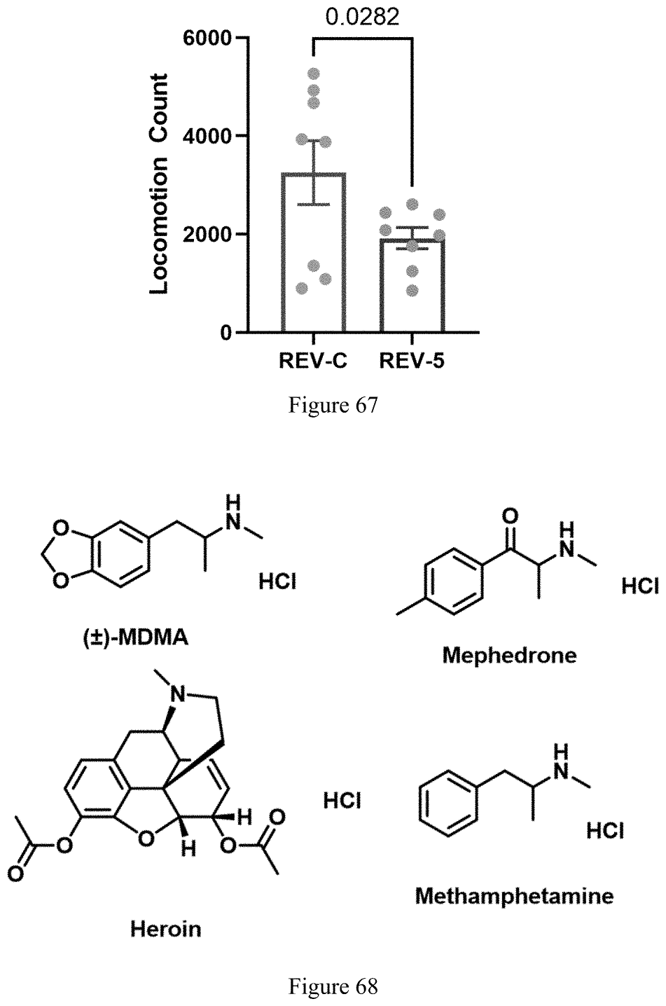

Although the results of this first analysis are suggestive of the potential efficacy of P[6]AS in the sequestration of methamphetamine and inducing behavioral change, it is possible that the 30 second (s) interval between methamphetamine administration and P[6]AS administration in the reversal condition is too short to be ethologically relevant. To address this, a follow up experiment was conducted where on days 7 and 8 of testing, mice (n=8) were administered either methamphetamine followed by administration of 0.01 M PBS 5 minutes later (REV-C) or methamphetamine followed by P[6]AS 5 minutes later (REV-5) in a counterbalanced manner before completing the autoshaping task. FIG. 67 plots locomotion counts as a function of either REV-C or REV-5 treatment. A significant decrease was observed in locomotion in the REV-5 condition relative to REV-C(paired t-test, t(7)=2.757, p=0.0282). Although not directly comparable from an experimental design perspective, importantly locomotion levels in the REV-5 condition closely approximate those observed in control conditions on Day 1-6, while locomotion counts in the REV-C condition appear to approximate those observed with the methamphetamine only treatment. Collectively these findings suggest that P[6]AS is capable of sequestering methamphetamine in vivo and reversing methamphetamine-induced hyperlocomotion, with little to no effect on the locomotor behavior of the animal itself.

### Example 4

The following example shows ITC data for the binding of various drugs with hosts of the present disclosure.

TABLE 10
Kₐ of MDMA, mephedrone, and heroin.

| | Kₐ with P[6]AS (M⁻¹) |
|---|---|
| Drug | ΔH, C or app. C |
| MDMA | (3.92 ± 0.20) × 10^{7 a} |
| | −13.30 ± 0.04 |
| | 49.8 |
| Mephedrone | (1.91 ± 0.19) × 10^{7 b} |
| | −12.60 ± 0.11 |
| | 191 |
| Heroin | (5.78 ± 0.02) × 10^{5 c} |
| | −11.9 ± 0.11 |
| | 57.8 |

^{a} Measured by the ITC competition titration of Host (0.1 mM) and 1,3-propanediammonium chloride (0.15 mM) in the cell with Guest (1 mM) in the syringe.
^{b} Measured directly by the ITC titration of Host (10 μM) in the cell with Guest (100 μM) in the syringe.
^{c} Measured directly by the ITC titration of Host (0.1 mM) in the cell with Guest (1 mM) in the syringe.

FIGS. 69-71 shows ITC data of P[6]AS and MDMA, mephedrone, and heroin.

### Example 5

The following example shows use of pillararene sulfates as in vivo reversal.

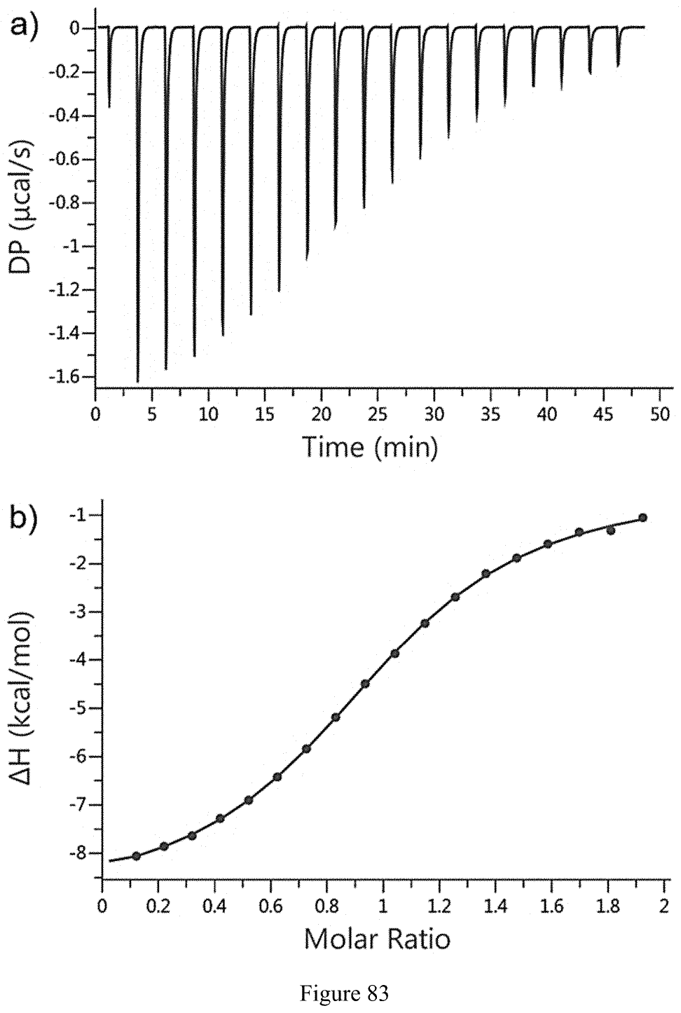

Human ether-a-go-go (hERG) Ion Channel Inhibition Assay. The hERG ion channel is a voltage-gated potassium channel in cardiac cells that is essential for cardiac repolarization. With the inhibition of this channel, the electrical depolarization and repolarization of the heart ventricles can be extended, leading to potentially fatal cardiac malfunction. The ability of P6AS at six concentrations (0.008 μM to 25 μM) to inhibit the hERG ion channel function was evaluated via the patch-clamp technique (QPatch HTX). The patch clamp hERG assay was conducted using mammalian cells (HEK-293) expressing the hERG channel. FIG. 83 shows the results of the hERG assay for P6AS and for E-4031 as positive control. As can be readily seen, the positive control (E-4031) exhibits a sharp increase in inhibition of ion channel activity as the concentration increases past 0.01 μM. In contrast, no concentration dependent change in ion channel activity is observed for the cells treated with P6AS. The calculated IC₅₀ value for E-4031 is 0.0267 μM whereas the IC₅₀ value for P6AS is greater than 25 μM. IC50 values below 0.1 μM are defined as highly potent inhibitors of the hERG channel, values between 0.1 and 1 μM as potent, values between 1-10 μM as moderately potent, and finally, IC₅₀ values above 10 μM are typically categorized as having little to no inhibition of the channel. Accordingly, P6AS is not an inhibitor of the hERG ion channel which encourages the further development of the in vivo sequestering abilities of the compound.

Ames Test. In order to assess the potential mutagenicity of P6AS, the Ames fluctuation test and the associated bacterial cytotoxicity assays were performed. The Ames fluctuation test is a reverse mutation assay that utilizes four different S. typhimurium strains (TA98, TA100, TA1535, TA1537) which possess unique mutations within the histidine operon. Compounds that induce reverse mutations allow these strains to grow in the absence of histidine which is measured spectroscopically. The S. typhimurium strain TA1535 contains a T to C missense mutation in the hisG gene (his G46) leading to a leucine to proline amino acid substitution. With the reversal of this mutation, TA1535 can detect compounds that cause base pair mutations. The TA1537 strain detects compounds that induce a +1 frameshift mutation on the his C gene (his C3076). This allows frameshift mutagens to be detected. The TA98 strain detects +1 frameshift mutation on the his D gene (his D3052) and also contains the pkM101 plasmid, which increases the sensitivity of the strain to mutagenic compounds. Finally, TA100 contains the same mutation as TA1535 plus the pkM101 plasmid. The Ames fluctuation test also employs rat liver enzyme fractions (S9) to assess the potential mutagenicity of metabolites produced by the action of the liver enzymes on the test compound.

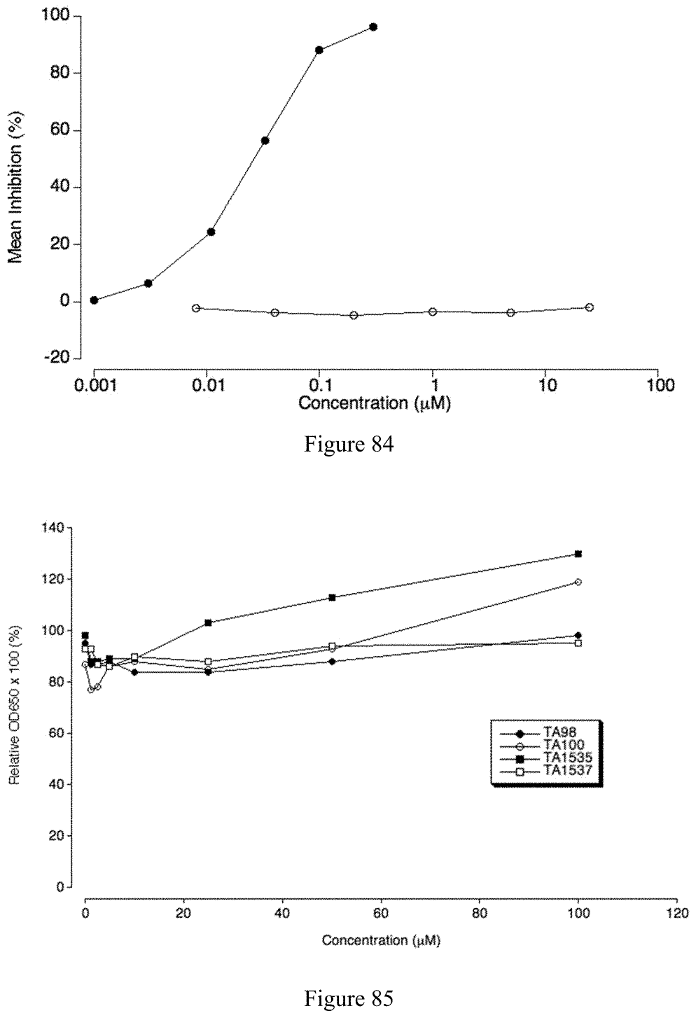

Initially, bacterial cytotoxicity assays were performed to determine whether P6AS was cytotoxic toward the histidine revertant tester strains (TA98R, TA100R, TA1535R, TA1537R) which would cause false negatives in the Ames fluctuation test. For this purpose, the four tester strains were cultured overnight at 37° C. in media containing Davis Mingoli salts, D-glucose, D-biotin, and low level histidine at pH 7.0 yielding OD₆₅₀ from 0.60 to 1.10. The cultures were then incubated with eight different concentrations of P6AS (0.6, 1.2, 2.5, 5, 10, 25, 50, 100 μM; n=3) for 96 hours followed by measurement of OD₆₅₀. Compounds that exhibit OD₆₅₀ values less than 60% of control (not treated with compound) are deemed cytotoxic and do not proceed to the Ames fluctuation test. The known cytotoxic compound mitomycin C (IC₅₀≤100 nM toward the tester strains) is used as a positive control. P6AS did not exhibit bacterial cytotoxicity toward any of the four tester strains at concentrations up to 100 μM (FIG. 84).

Given the absence of bacterial cytotoxicity for P6AS, the Ames fluctuation test was subsequently performed. For this purpose, the four tester strains of bacteria were cultured overnight in media containing Davis Mingoli salts, D-glucose, D-biotin, and low level histidine at pH 7.0 yielding OD₆₅₀ from 0.60 to 1.10. The cultures were then incubated in the absence of P6AS or in the presence of P6AS (5, 10, 50, 100 μM; n=48) both with and without Arochlor-induced rat liver S9 fraction (0.2 mg mL⁻¹) for 96 hours. Bromocresol purple is included as a colorimetric pH indicator that responds to the pH drop resulting from bacterial growth upon reverse mutation. After 96 hours, the OD₄₃₀ and OD₅₇₀ values are measured and the number of positive wells with OD₄₃₀/OD₅₇₀≥1 is determined as surrogate for reverse mutation. The significance of the number of positive wells in the treatment groups (P6AS present) versus the control group (P6AS absent) is calculated using the one-tailed Fisher's exact test and classified as follows: p<0.001 (very strong positive, +++); 0.001<p <0.01 (strong positive, ++); 0.01<p<0.05 (weak positive, +); p >0.05 (negative, −). Control compounds known to induce reverse mutation (2-aminoanthracene (2-AA), 9-aminoacridine (9-AA), Quercetin, Streptozotocin) were tested as positive controls. Table 11 presents the results of the Ames fluctuation test. As can be seen, compared to background, none of the P6AS treatments result in a statistically significant increase in the number of positive wells. This indicates that P6AS does not significantly increase the rate of reverse mutation and is not genotoxic. Conversely, the genotoxic control compounds Streptozotocin, 2-AA, Quercetin, and 9-AA all display the expected increase in genotoxicity in one or more bacterial strains.

TABLE 11
Results from the Ames fluctuation test conducted for P6AS.

| | TA98 | | TA100 | | TA1535 | | TA1537 | |
|---|---|---|---|---|---|---|---|---|
| | Treatment | | | | | | | |
| | −S9 | +S9 | −S9 | +S9 | −S9 | +S9 | −S9 | +S9 |
| Background | 0/48 | 1/48 | 0/48 | 4/48 | 0/48 | 0/48 | 1/48 | 0/48 |
| [P6AS] = 5 μM | 0/48 | 2/48 | 0/48 | 0/48 | 0/48 | 0/48 | 0/48 | 1/48 |
| | − | − | − | − | − | − | − | − |
| [P6AS] = 10 | 0/48 | 0/48 | 0/48 | 0/48 | 0/48 | 0/48 | 0/48 | 2/48 |
| μM | − | − | − | − | − | − | − | − |
| [P6AS] = 50 | 0/48 | 0/48 | 0/48 | 1/48 | 1/48 | 0/48 | 0/48 | 0/48 |
| μM | − | − | − | − | − | − | − | − |
| [P6AS] = 100 | 1/48 | 2/48 | 0/48 | 1/48 | 0/48 | 0/48 | 0/48 | 0/48 |
| μM | − | − | − | − | − | − | − | − |
| Streptozotocin | 0/48 | 0/48 | 5/48 | 7/48 | 16/48 | 24/48 | 1/48 | 1/48 |
| | − | − | + | − | +++ | +++ | − | − |
| 2-AA | 0/48 | 13/48 | 0/48 | 11/48 | 0/48 | 9/48 | 0/48 | 6/48 |
| | − | +++ | − | + | − | ++ | − | + |
| Quercetin | 5/48 | 10/48 | 0/48 | 5/48 | 1/48 | 0/48 | 1/48 | 5/48 |
| | + | +++ | − | − | − | − | − | + |
| 9-AA | 0/48 | 0/48 | 0/48 | 2/48 | 0/48 | 0/48 | 24/48 | 24/48 |
| | − | − | − | − | − | − | +++ | +++ |

In Vivo Reversal of Fentanyl Induced Hyperlocomotion by P6AS—Experiment #1.

Animals. Nine male Swiss Webster (CFW) mice were obtained from Charles River Laboratories that weighed ˜35 g upon arrival. Mice were individually housed in a temperature- and humidity-controlled room on a 12 h (hour) light/dark schedule with lights on at 6:00 am EST. For the duration of both experiments mice had ad libitum access to food and water. All behavioral testing occurred between 6:30 am and 2:00 pm EST, and all experimental procedures were approved by the University of Maryland Animal Care and Use Committee and conformed to the guidelines set forth by the National Research Council.

Surgical Procedures. Mice were anesthetized with an intraperitoneal (IP) injection of ketamine (100 mg/kg)/xylazine (10 mg/kg) (n=9) and were implanted with jugular catheters with head-mounted ports. All surgical procedures were conducted using aseptic technique, with body temperature monitored and maintained throughout surgery. Catheters were placed in the right jugular vein with the port passed subcutaneously out towards the top of skull. Ports (5 MM Up Pedestal; P1 Technologies) were fixed to the skull with a combination of super glue (Loctite) and dental cement. Following surgery, mice received an immediate injection of Rimadyl (5 mg/kg) and 0.4 mL of warm sterile saline. Mice were treated post-operatively for two days with Rimadyl (5 mg/kg) and given a minimum of 5 days to recover before testing. Catheters were flushed daily with 0.1 mL sterile saline solution containing gentamycin (0.33 mg/mL) and 0.1 mL sterile saline solution containing heparin (20 IU/mL) in order to reduce clotting and maintain catheter patency. Catheter patency was assessed daily from the first day following surgery till the end of testing. Any mouse whose catheter exhibited significant flowback on a majority of days was excluded from analysis.

Behavioral Testing. Mice were trained on a standard autoshaping task. All behavioral procedures were conducted in a Med Associates test chamber equipped with a food cup, a retractable lever, and 4 floor IR photobeams. Time stamps were generated from head entries into the food cup, downward deflections of the lever, or disruption of floor beams and recorded by the behavioral computer.

In order to minimize the impact of novelty-induced suppression of feeding, mice were given five to six 20 mg sucrose pellets (Bioserv) each in their home cage for 2-3 days prior to the beginning of training. Mice were weighted and handled daily upon arrival until the completion of testing.

Following surgery, mice were habituated to the behavioral box and underwent one session of autoshaping to establish baseline locomotion levels before treatment began. Pavlovian training sessions which consisted of the presentation of the lever (CS) for 8 s, which was immediately followed by the delivery of a sucrose pellet and the retraction of the lever. The CS was presented on a random interval of 90±30 s schedule. Each Pavlovian session consisted of 30 trials. In total baseline plus testing lasted 11 days.

Experimental Design. P6AS efficacy was assessed using a semi-counterbalanced design where all mice received each possible experimental treatment. The purpose of the experiments was to: (1) verify that binding of fentanyl by P6AS would not be compromised in vivo, (2) verify that P6AS would not alter locomotor behavior, and (3) to demonstrate that P6AS can sequester fentanyl in vivo. On the first day of testing, regardless of experiment, mice underwent a baseline session free of treatment. On the following six sessions mice were treated with one of six possible treatments: PBS (0.2 mL infused), P6AS only (4 mM; 0.178 mL infused), fentanyl only (0.17 mg/mL; 0.022 mL infused), a premixed solution of P6AS and fentanyl (Premix; ˜68.34:1 P6AS:Fentanyl; 0.178 mL P6AS+0.022 mL fentanyl infused), P6AS followed by fentanyl administered 30 s later (0.178 mL P6AS, 0.022 mL fentanyl infused), and fentanyl followed by P6AS administered 30 s later (0.022 mL fentanyl, 0.178 mL P6AS infused). Mice received only one treatment per day. The dose of fentanyl was chosen based on previously published values that observed reliable hyperlocomotion in mice. The smallest dosage that reliably induced hyperlocomotion was selected.

Following completion of the first six sessions, mice completed another two days of behavioral testing. On day 8, half of the mice (n=5) received fentanyl followed by P6AS administered 5 minutes later (0.022 mL fentanyl infused, 0.178 mL P6AS), followed by infusion of and fentanyl followed by PBS administered 5 minutes later (0.022 mL fentanyl, 0.178 mL PBS infused) administered on the ninth day of testing. The other half of the mice (n=4) received the same exact treatment but in reverse order across days 8 and 9. On days 10 and 11, mice received the same treatment order but the time between fentanyl and PBS or P6AS was extended to 15 minutes.

For each experiment, total locomotion counts (i.e., the total number of beam breaks) were obtained for each mouse across the entirety of each training session. For each experiment, locomotion counts were then analyzed across treatments using one-way repeated measures ANOVAs with tukey-corrected pairwise post-hoc t-tests in Graphpad Prism (Version 9.0.0). For 5 minute and 15-minute reversal experiments data were analyzed with paired t-tests.

Results.

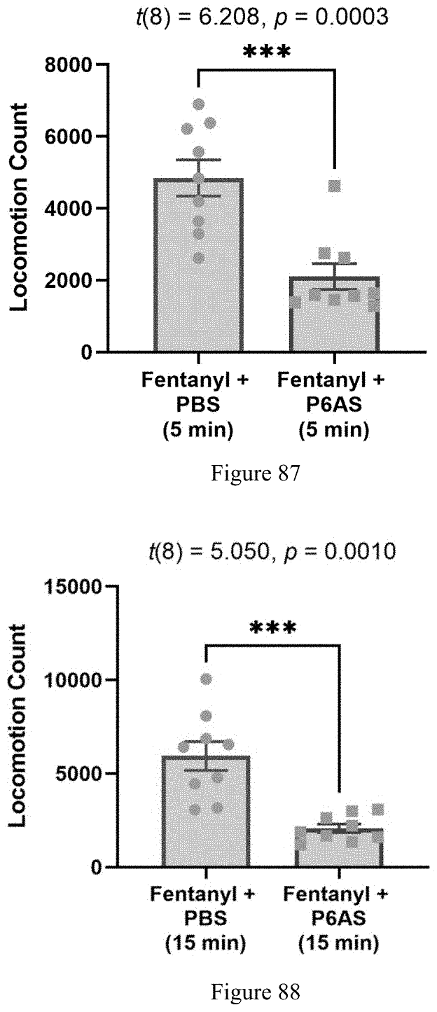

Applicable to FIGS. 85, 86, and 87: The concentrations of compounds used for the injections were selected so that the doses were as follows: P6AS (4.0 mM)=35.8 mg/kg; Fentanyl=0.1766 mg/ml=0.1 mg/kg. The molar ratio of P6AS (15 mg/kg):fentanyl is 68.3:1.

Discussion.

The efficacy of P6AS in the sequestration of fentanyl was examined in vivo. Nine male Swiss Webster (CFW) mice were trained on an Pavlovian autoshaping task described previously and locomotion values were obtained and analyzed accordingly. To establish fentanyl induced hyperlocomotion and examine the efficacy of P6AS, mice were first treated single infusions of PBS (0.01 M), P6AS only, fentanyl only, a premixed solution of P6AS and fentanyl, P6AS followed by fentanyl administration 30s later, or fentanyl followed by P6AS administered 30s later in counterbalanced manner. FIG. 85 depicts the results of this experiment by plotting locomotion counts as a function of treatment. Mixed effects analysis revealed a significant main effect of treatment (F(8,48)=22.47, p<0.0001) with Tukey-corrected post-hoc comparison showing a significant increase in locomotion counts for treatment with fentanyl against all other treatments (p's<0.05).

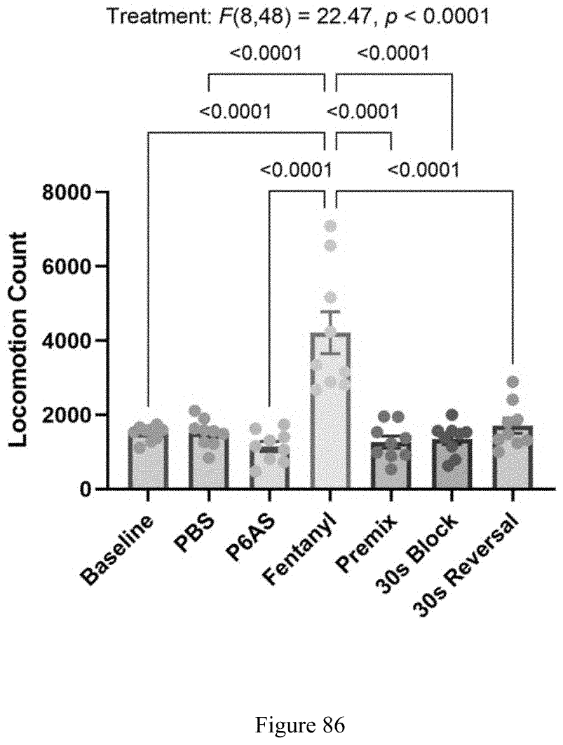

Although the results of this first analysis are suggestive of the potential efficacy of P6AS in the sequestration of fentanyl and inducing behavioral change, it is possible that the 30s interval between fentanyl administration and P6AS administration in the reversal condition is too short to be ethologically relevant. To address this, two follow up experiment were conducted, where on days 8/9 of testing, and days 10/11 of testing, mice (n=9) were administered either fentanyl followed by administration of 0.01M PBS or fentanyl followed by P6AS either 5 or 15 minutes later in a counterbalanced manner before completing the autoshaping task. FIG. 86 plots locomotion counts as a function of either treatment for the 5-minute experiment. A significant decrease in locomotion was observed when P6AS was administered 5 minutes after fentanyl administration condition relative to when PBS was administered 5 minutes later (paired t-test, t(8)=6.208, p=0.0003). Similarly, administration of P6AS 15 minutes after fentanyl administration significantly reduced hyperlocomotion in comparison to PBS treatment (paired t-test, t(8)=5.050, p=0.0010).

Although not directly comparable from an experimental design perspective, locomotion levels in the 5-minute reversal and 15-minute reversal using P6AS condition closely approximated those observed in control conditions on Day 1-7, while locomotion counts in the PBS condition approximate those observed with the fentanyl only treatment. Collectively these findings suggest that P6AS is capable of sequestering fentanyl in vivo and reversing fentanyl-induced hyperlocomotion, with little to no effect on the locomotor behavior of the animal itself.

Given the desirable results obtained in these experiments, it was tested whether lower doses of P6AS would be similarly effective and also included active comparators in the form of Naloxone and Motor1.

Comparison of Lower Doses of P6AS, Naloxone, and TetM1 as Countermeasures to Fentanyl-Induced Hyperlocomotion in Mice—Experiment #2.

Animals. Eleven male Swiss Webster (CFW) mice were obtained from Charles River Laboratories weighting 32.36±1.2 g upon arrival. Mice were individually housed in a temperature- and humidity-controlled room on a 12 h light/dark schedule with lights on at 6:00 am EST. For the duration of the experiment mice were given ad libitum access to food and water. All behavioral testing occurred between 6:00 am and 2:00 pm EST, and all experimental procedures were approved by the University of Maryland Animal Care and Use Committee and conformed to the guidelines set forth by the National Research Council.

Surgical Procedures. Mice were anesthetized with an intraperitoneal (IP) injection of ketamine (100 mg/kg)/xylazine (10 mg/kg) (n=11) and were implanted with jugular catheters with head-mounted ports. All surgical procedures were conducted using aseptic technique, with body temperature monitored and maintained throughout surgery. Catheters were placed in the right jugular vein with the port passed subcutaneously out towards the top of skull. Ports (5 MM Up Pedestal; P1 Technologies) were fixed to the skull with a combination of super glue (Loctite) and dental cement. Following surgery, mice received an immediate injection of Rimadyl (5 mg/kg) and 0.4 mL of warm 0.9% sterile saline. Mice were treated post-operatively for two days with Rimadyl (5 mg/kg) and given a minimum of 7 days to recover before testing. Catheters were flushed daily with 0.1 mL 0.9% sterile saline solution containing gentamycin (0.33 mg/mL) and 0.1 mL sterile saline solution containing heparin (20 IU/mL) in order to reduce clotting and maintain catheter patency. Catheter patency was assessed daily from the first day following surgery until the end of testing. Any mouse whose catheter exhibited significant flowback on a majority of days was excluded from analysis.

Behavioral Testing. Mice were trained on a standard autoshaping task described previously. All behavioral procedures were conducted in a Med Associates test chamber equipped with a food cup, a retractable lever, and 4 floor IR photobeams. Time stamps were generated from head entries into the food cup, downward deflections of the lever, or disruption of floor beams and recorded by the behavioral computer.

In order to minimize the impact of novelty-induced suppression of feeding, mice were given five to six 20 mg sucrose pellets (Bioserv) each in their home cage for 2-3 days prior to the beginning of training. Mice were weighted and handled daily upon arrival until the completion of testing.

Following surgery, mice were habituated to the behavioral box and underwent one session of autoshaping to establish baseline locomotion levels before treatment began. Pavlovian training sessions which consisted of the presentation of the lever (CS) for 8 s, which was immediately followed by the delivery of a sucrose pellet and the retraction of the lever. The CS was presented on a random interval of 90±30 s schedule. Each Pavlovian session consisted of 30 trials. In total baseline plus testing lasted 11 days.

Experimental Design. P6AS efficacy was assessed using a semi-counterbalanced design where all mice received each possible experimental treatment. The purpose of the experiments was to: (1) verify that binding of fentanyl by P6AS would not be compromised in vivo, (2) test lower doses of P6AS (0.5 mM and 1.5 mM) than previously used (4.0 mM) to modulate locomotor behavior, and (3) to compare the efficacy of P6AS sequester fentanyl to alternative countermeasures, naloxone (4.37 mM) and TetM1 (1.507 mM), in vivo. On the first day of testing, regardless of experiment, mice underwent a baseline session free of treatment. During the following six sessions mice underwent 15 minute reversals where either a single 0.022 mL infusion of PBS or 0.1591 mg/mL fentanyl followed by a 0.178 mL infusion of a candidate countermeasure. The possible six treatments included PBS followed by PBS, fentanyl followed by 1.5 mM P6AS, fentanyl followed by 0.5 mM P6AS, fentanyl followed by 4.37 mM naloxone, fentanyl followed by 1.507 mM TetM1, or fentanyl followed by PBS. Mice received only one treatment per day, and treatments were counterbalanced across animals. The dose of fentanyl used was chosen based on previously published values that observed reliable hyperlocomotion in mice.

TetM1 is Also Known as Motor 1 or Calabadion 1.

Total locomotion counts (i.e., the total number of beam breaks) were obtained for each mouse across the entirety of each behavioral session. Locomotion counts were analyzed across treatments using one-way repeated measures ANOVAs with tukey-corrected pairwise post-hoc t-tests in Graphpad Prism (Version 9.0.0).

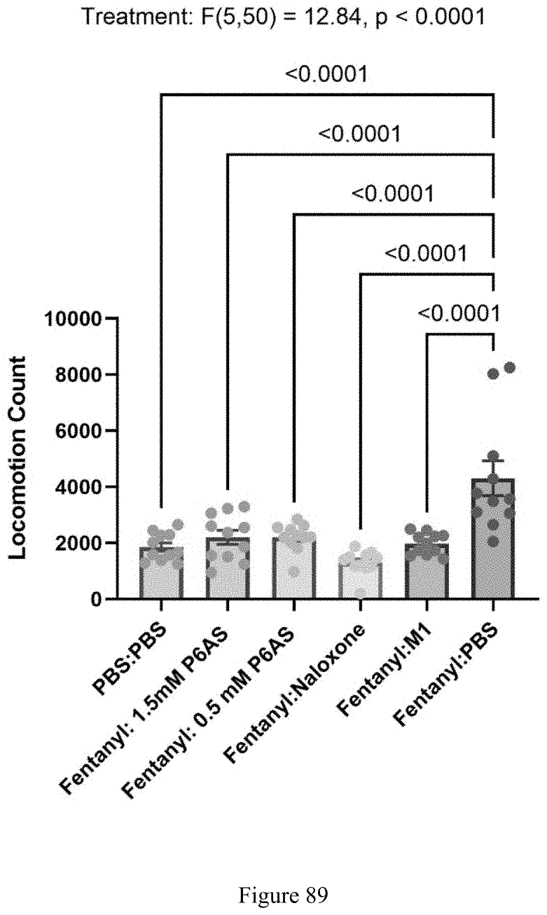

Discussion The efficacy of P6AS in the sequestration of fentanyl was investigated, as well as comparison its efficacy to other known countermeasures in vivo. Eleven male Swiss Webster (CFW) mice were trained on an Pavlovian autoshaping task and locomotion values were obtained and analyzed accordingly. Mice underwent 15 minutes reversals in which they were first treated with either PBS or 0.1591 mg/mL of fentanyl before receiving one of six possible countermeasures 15 minutes later. FIG. 89 depicts the results of this experiment by plotting locomotion counts as a function of treatment. Mixed effects analysis revealed a significant main effect of treatment (F(5,50)=12.84, p<0.0001) with Tukey-corrected post-hoc comparison showing a significant increase in locomotion counts for treatment with fentanyl then PBS against all other countermeasure combinations (p's<0.05).

The results establish that P6AS is a desirable in vivo sequestrant for fentanyl that is capable of reversing the hyperlocomotion observed for animals treated with fentanyl. Reversal using P6AS, TetM1, and Naloxone displayed no statistically significant differences. The fact that low dose P6AS (5 mg/kg) is capable of effecting reversal is a finding with translational potential.

Although the present disclosure has been described with respect to one or more particular example(s), it will be understood that other examples of the present disclosure may be made without departing from the scope of the present disclosure.

1. A compound having the following structure: 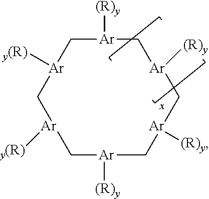
wherein
Ar is an aryl group wherein the aryl groups are attached in a para orientation to the adjacent methylene groups;
each R is independently chosen from: —OS(O)₂O⁻M⁺, —OS(O)₂OH, non-sulfate anionic groups, carboxylic acid/carboxylate groups, phosphonic acid/phosphonate groups, phosphate groups, substituted or unsubstituted aryl groups, substituted or unsubstituted heteroaryl groups, substituted or unsubstituted aliphatic groups, O-alkyl groups, —H, substituted or unsubstituted alkyl groups, halogens, amide groups, cyano groups, substituted or unsubstituted sulfur-containing aliphatic groups, nitro groups, amino groups, substituted or unsubstituted nitrogen-containing aliphatic groups, substituted or unsubstituted polyethylene glycol groups, polyether groups, O-aryl groups, ester groups, carbamate groups, imine groups, aldehyde groups, —SO₃H groups, —SO₃Na groups, —OSO₂F groups, —OSO₂CF₃ groups, —OSO₂OR′″ groups, wherein R′″ are substituted or unsubstituted aryl groups or substituted or unsubstituted alkyl groups, and combinations thereof, wherein M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS),
x is 0, 1, 2, or 3; and y is independently at each occurrence 0, 1, 2, 3, or 4, with the proviso that at least one y is 1 and at least one R group is —OS(O)₂O⁻M⁺, wherein M⁺ is Na⁺, K⁺, Ca²⁺, Mg²⁺, Zn²⁺, H₄N⁺, Et₃NH⁺, Me₄N⁺, (HOCH₂CH₂)₃NH⁺, or a cationic form of ethylenediamine, piperazine, or trishydroxymethyl aminomethane (TRIS) or —OS(O)₂OH, or a salt, a partial salt, a hydrate, a polymorph, a stereoisomer, conformational isomer, or a mixture thereof.

2. The compound of claim 1, wherein the aryl groups are independently at each occurrence chosen from phenyl groups, fused-ring groups, biaryl groups, and terphenyl groups.

3. The compound of claim 1, wherein at least two of the one or more phenyl group(s) of one or more of the aryl group(s) comprising the cyclic core of the compound have at least 1 R groups independently chosen from —OS(O)₂O⁻M⁺ and —OS(O)₂OH.

4. The compound of claim 3, wherein the compound has the following structure:

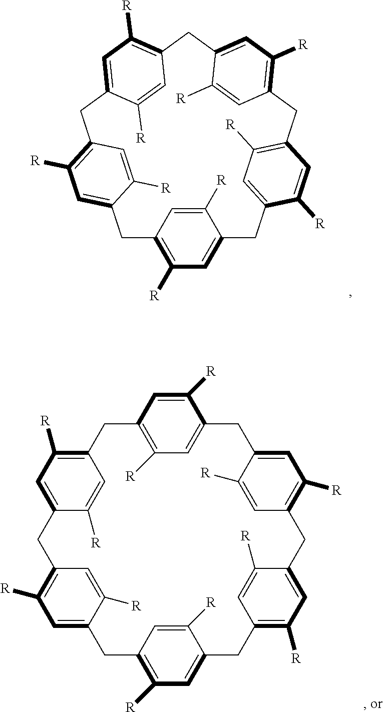

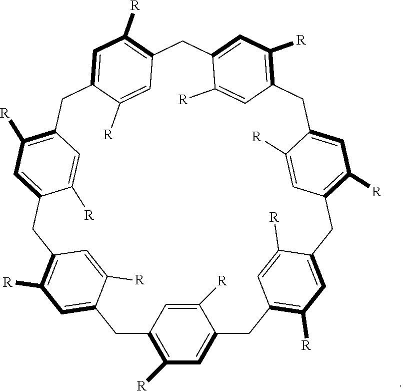

5. The compound of claim 1, wherein all of the aryl groups comprise an R group that is independently —OS(O)₂O⁻M⁺ or —OS(O)₂OH.

6. The compound of claim 1, wherein at least one aryl group does not comprise an R group that is —OS(O)₂O⁻M⁺ or —OS(O)₂OH.

7. The compound of claim 1, wherein the compound has the following structure:

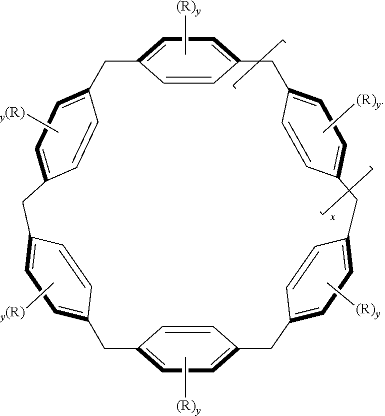

8. The compound of claim 7, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of the R groups are independently —OS(O)₂O⁻M⁺ groups or —OS(O)₂OH groups.

9. The compound of claim 7, wherein each phenyl group comprising the cyclic core of the compound has at least 1 R group independently chosen from —OS(O)₂O⁻M⁺ and —OS(O)₂OH.

10. The compound of claim 7, wherein at least one phenyl group does not comprise an R group that is —OS(O)₂O⁻M⁺ or —OS(O)₂OH.

11. The compound of claim 1, wherein M⁺ is Na⁺, K⁺, H₄N⁺, Et₃NH⁺, Me₄N⁺, (HOCH₂CH₂)₃NH⁺.

12. The compound of claim 11, wherein M⁺ is Na⁺.

13. A composition comprising one of more compound(s) of claim 1.

14. The composition of claim 13, further comprising a pharmaceutical carrier.

15. The composition of claim 13, wherein the one or more compound(s) are disposed on at least a portion of a solid substrate.

16. The composition of claim 15, wherein the solid substrate comprises silica, polymer beads, polymer resins, metal nanoparticles, a metal, or a combination thereof.

17. The composition of claim 13, wherein at least a portion or all of the one or more compound(s) have a pharmaceutically active agent(s) disposed in a cavity of the one or more compound(s).

18. A method for sequestering one or more neuromuscular blocking agent(s), one or more anesthesia agent(s), one or more pharmaceutical agent(s), one or more pesticide(s), one or more dyestuff(s), one or more malodorous compound(s), one or more chemical warfare agent(s), one or more hallucinogen(s), one or more toxin(s), one or more metabolite(s), or a combination thereof comprising:
contacting the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof with one or more compound(s) of claim 1, wherein the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof are sequestered by the one or more compound(s).

19-20. (canceled)

21. The method of claim 18, wherein a complex is formed from the one or more compound(s) and the neuromuscular blocking agent(s), the anesthesia agent(s), the pharmaceutical agent(s), the pesticide(s), the dyestuff(s), the malodorous compound(s), the chemical warfare agent(s), one or more hallucinogen(s), one or more toxin(s), one or more metabolite(s), or a combination thereof.

22-24. (canceled)

25. A method for reversing drug-induced neuromuscular block and/or anesthesia and/or the effects of one or more pharmaceutical agent(s) in an individual comprising administering to an individual in need of reversal of neuromuscular block and/or reversal of anesthesia and/or reversal of the effects of one or more pharmaceutical agent(s) one or more compound(s) of claim 1.

26-29. (canceled)

30. The method of claim 25, wherein the individual is in need of reversal of the effects of one or more pharmaceutical agent(s) and the one or more pharmaceutical agent(s) are chosen from one or more drug(s) of abuse, one or more pesticide(s), one or more chemical warfare agent(s), one or more nerve agent(s), one or more hallucinogen(s), one or more toxin(s), one or more metabolite(s), and combinations thereof.

31-32. (canceled)

33. The method of claim 30, wherein the drug of abuse is fentanyl.

34. The method of claim 33, wherein the one or more compound(s) are administered at least five minutes after administration of the fentanyl.

35. A method for prophylaxis and/or therapy of a condition in an individual comprising administering to an individual in need of the prophylaxis and/or the therapy one or more compound(s) of claim 1 and one or more pharmaceutical agent(s), wherein the compound(s) and the pharmaceutical agent(s) are present as complex, wherein subsequent to the administration the therapy and/or the prophylaxis of the condition in the individual occurs.

36. (canceled)
